(19) United States
(12) Patent Application Publication
(10) Pub. No.: US 2008/0119396 A1
(43) Pub. Date: May 22, 2008

(54) Tgf Derepressors and Uses Related Thereto

(75) Inventors: John Knopf, Carlisle, MA (US); Jasbir Seehra, Lexington, MA (US)

Correspondence Address: ROPES & GRAY LLP, PATENT DOCKETING 39/41, ONE INTERNATIONAL PLACE, BOSTON, MA, 02110-2624, US

(73) Assignee: Acceleron Pharma Inc., Cambridge, MA (US)

(21) Appl. No.: 11/597,096

(22) PCT Filed: May 27, 2005
(86) PCT No.: PCT/US05/18911

§ 371 Date: Oct. 31, 2007

Related U.S. Application Data
(60) Provisional application No. 60/575,067, filed on May 27, 2004.

(51) Int. Cl. A61K 38/00; C07K 14/51; A61P 43/00

(52) U.S. Cl. 514/11; 530/399; 530/326

(57) ABSTRACT

The application is directed to TGF analogs/derepressors that bind to and neutralize cystine knot-containing BMP antagonists—such as the CAN subfamily of Cystine-knot proteins including sclerostin. The subject TGF derepressors can be prepared as substantially pyrogen-free pharmaceutical compositions for administration to mammals, in treating diseases such as bone diseases including osteoporosis, and any conditions with lesser-than-desired amount of BMP activity.

### BACKGROUND OF THE INVENTION

Transforming growth factor-β superfamily proteins represent a large family of cytokines that includes the TGF-βs, activins, inhibins, bone morphogenetic proteins (BMPs) and Mullerian-inhibiting substance (MIS) (for review, see Massague et al., Trends Cell Biol. 7:187-192, 1997). These proteins contain a conserved C-terminal cystine-knot motif, and serve as ligands for diverse families of plasma membrane receptors. Members of the TGF-β family exert a wide range of biological effects on a large variety of cell types. For example, they regulate cell growth, differentiation, matrix production and apoptosis. Many of them have important functions during embryonal development in pattern formation and tissue specification; in the adult, they are involved in processes such as tissue repair and modulation of the immune system.

Activities of the TGF-β superfamily proteins are regulated through various means. The BMP subfamily of proteins is negatively regulated by a large family of so-called Bone Morphogenetic Protein (BMP) antagonists/repressors. These BMP repressors represent a subgroup of proteins that bind BMPs, and interfere with BMP binding to their membrane receptors, thereby antagonizing their actions during development and morphogenesis.

The BMP repressors can be further divided into three groups of proteins based on structural analysis, and particularly based on the number of structurally conserved Cys residues in their C-terminal characteristic “Cystine-knot” structures: the 8-, 9-, or 10-member ring Cystine-knot BMP repressors. One of the 8-member ring repressors is Sclerostin (SOST), which is known to be involved in a disease condition known as Sclerosteosis.

Sclerosteosis is a term that was applied by Hansen (Sklerosteose. In: Opitz, H.; Schmid, F.: Handbuch der Kinderheilkunde. Berlin: Springer (pub.) 6 1967. Pp. 351-355) to a disorder similar to van Buchem hyperostosis corticalis generalisata but differing in radiologic appearance of the bone changes and in the presence of asymmetric cutaneous syndactyly of the index and middle fingers in many cases. The jaw of a patient has an unusually square appearance in this condition. Affected siblings were observed by Hirsch (Radiology 13: 44-84, 1929), Falconer and Ryrie (Med Press 195: 12-20, 1937), Higinbotham and Alexander (Am. J. Surg. 53: 444-454, 1941), Kelley and Lawlah (Radiology 47: 507-513, 1946), Truswell (J. Bone Joint Surg. 40B: 208-218, 1958) and Klintworth (Neurology 13: 512-519, 1963). Parental consanguinity was observed by Falconer and Ryrie (Med. Press 195: 12-20, 1937) and by Truswell (J. Bone Joint Surg. 40B: 208-218, 1958).

Through a genome-wide search with highly polymorphic microsatellite markers, Van Hul et al. (Am J Hum Genet. 62(2): 391-9, 1998) mapped the gene responsible for van Buchem disease to 17q12-q21. Balemans et al. (Am. J. Hum. Genet. 64: 1661-1669, 1999) tested the hypothesis of Beighton et al. (Clin. Genet. 25: 175-181, 1984) that sclerosteosis and van Buchem disease may be caused by mutations in the same gene. By 2-point linkage analysis in 2 consanguineous families with sclerosteosis, Balemans et al. (Am. J. Hum. Genet. 64: 1661-1669, 1999) assigned the locus for this disease to 17q12-q21, the same region where the van Buchem disease locus maps, providing genetic support for the hypothesis of allelism.

Through homozygosity mapping followed by positional cloning in Afrikaner families with sclerosteosis, Brunkow et al. (Am J Hum Genet. 68(3): 577-89, 2001; Epub 2001 Feb. 9) found 2 independent mutations in a novel gene, which they termed SOST. The SOST gene contains 2 exons encoding a deduced 213-amino acid protein, sclerostin, that shares 89% and 88% sequence identity with the rat and mouse homologs. The protein contains a putative secretion signal and 2 N-glycosylation sites. It also contains a cystine knot motif (residues 80-167) with high similarity to the dan family of secreted glycoproteins, including dan, cerberus, gremlin, and caronte, which have been shown to act as antagonists of members of the transforming growth factor-beta superfamily. Quantitative RT-PCR showed relatively low overall expression of SOST, but significant expression in whole long bone, cartilage, kidney, and liver and lower expression in placenta and fetal skin.

In affected Afrikaners, Brunkow et al. found a nonsense mutation in the N-terminus of sclerostin. In an unrelated affected person of Senegalese origin reported by Tacconi et al. (Clin. Genet. 53: 497-501, 1998), they found a splice mutation within the single intron of the SOST gene. Brunkow et al. analyzed the SOST gene in 7 Dutch patients with van Buchem disease and detected no mutations in the coding region.

Balemans et al. (Hum Mol. Genet. 10(5): 537-43, 2001) independently isolated the SOST gene and described 2 sclerosteosis families harboring mutations. Quantitative RT-PCR experiments revealed highest tissue expression level in control human kidney, followed by bone marrow and osteoblasts.

As mentioned above, the SOST gene product sclerostin shares some sequence similarity with a class of cystine knot-containing factors including dan, cerberus, gremlin, prdc, and caronte. The sclerostin protein gene is thought to interact with one or more of the bone morphogenetic proteins (BMPs) (Brunkow et al, 2001). Bone morphogenetic proteins are members of the transforming growth factor (TGF-β) superfamily that have been shown to play a role in influencing cell proliferation, differentiation and apoptosis of many tissue types including bone. Bone morphogenetic proteins can induce de novo cartilage and bone formation, and appear to be essential for skeletal development during mammalian embryogenesis (Wang 1993). Early in the process of fracture healing the concentration of bone morphogenetic protein-4 (BMP-4) increases dramatically (Nakase et al. 1994 and Bostrom et al. 1995). In vivo experiments indicate that up-regulation of BMP-4 transcription may promote bone healing in mammals (Fang et al. 1996). Bone morphogenetic proteins have been reported to induce the differentiation of cells of the mesenchymal lineage to osteogenic cells as well as to enhance the expression of osteoblastic phenotypic markers in committed cells (Gazzero et al. 1998, Nifuji & Noda, 1999). The activities of bone morphogenetic proteins in osteoblastic cells appear to be modulated by proteins such as noggin and gremlin that function as bone morphogenetic protein antagonists by binding and inactivating bone morphogenetic proteins (Yamaguchi et al. 2000).

Increased BMP activity can be explored for the treatment of a variety of disease conditions in which BMP activity is needed. For example, osteoporosis is a bone disorder characterized by the loss of bone mass, which leads to fragility and porosity of the bone of man. As a result, patients suffering from osteoporosis have an increased fracture risk of the bones. Postmenopausal women are particularly at risk for osteoporosis as a result of reduced levels of estrogen production. When administered at low levels, estrogens have a beneficial effect on the loss of bone. However, estrogen replacement therapy can have unwanted side effects including an increased risk of blood clots, breast carcinomas, endometrium hyperplasia, and an increased risk of endometrium carcinomas. The remaining current therapies provide little in terms of generating new bone for osteoporotic patients. Hence, a need exists for an alternative treatment of osteoporosis.

### SUMMARY OF THE INVENTION

One aspect of the invention provides a pharmaceutical preparation for derepressing (promoting) BMP signaling, e.g., receptor-mediated signaling by a member of the TGF/BMP family. Exemplary preparations of the subject invention include polypeptides comprising a mutant BMP analog that retains its ability to bind the cystine knot-containing BMP antagonists (also referred to herein as “cystine-knot family repressors”), but has diminished potency, relative to the wild-type BMP protein, for inducing receptor-mediated signal transduction in cells otherwise responsive to the wild-type TGF protein, such as by reducing its ability to bind to a type I and/or type II receptor. These so-called “BMP derepressors” can be used to reduce the severity of a pathologic condition, which is characterized, at least in part, by an abnormally low bone density in a subject. For instance, the pharmaceutical preparations of the present invention can be administered in an amount effective to prevent, ameliorate or reduce the severity of osteoporosis, such as post-menopausal osteoporosis.

Another aspect of the invention provides a pharmaceutical preparation for neutralizing the inhibitory activity of one or more Cystine-knot family proteins. Exemplary preparations of the subject invention include a Cystine-knot family inhibitor that binds to one of the Cystine-knot family proteins in a manner that inhibits binding of a Cystine-knot family protein to its normal binding partner, such as a BMP protein. Preferably, the Cystine-knot family inhibitor binds to the “BMP binding domain” of the Cystine-knot family protein.

In certain embodiments, the Cystine-knot family inhibitor/TGF derepressor is a mutant BMP polypeptide that includes a functional Cystine-knot family binding domain.

In certain embodiments, the TGF derepressor includes a dimerization domain mutation that prevents the formation of BMP dimers. In other embodiments, the TGF derepressor can be a heterodimer of a wild-type BMP monomer, and a mutant BMP monomer that has diminished ability to bind Type I or Type II or both BMP receptors, but has substantially unchanged ability to bind DAN family proteins.

Also included are variant sequences of the above-described TGF derepressors that may be desirable as a way to alter selectivity of the inhibitor (e.g., relative to one specific Cystine-knot family proteins), alter other binding characteristics with respect to Cystine-knot proteins (such as K_{d}, and/or Kₒₙ or K_{off} rates), or improve biodistribution or half life in vivo or on the shelf.

In certain preferred embodiments, the TGF derepressor binds Cystine-knot proteins with a K_{d} of 1 μM or less, and more preferably a K_{d} of 100 nM, 10 nM or even 1 nM or less.

In certain embodiments, the TGF derepressor is part of a fusion protein including one or more polypeptide portions that enhance one or more of in vivo stability, in vivo half life, uptake/administration, tissue localization or distribution, formation of protein complexes, and/or purification. For instance, the fusion protein can include an immunoglobulin Fc domain, preferably fused to the N-terminus of the TGF derepressor. The fusion protein may include a purification subsequence, such as an epitope tag, a FLAG tag, a polyhistidine sequence, or as a GST fusion.

In certain embodiments, the TGF derepressor is part of a protein that includes one or more modified amino acid residues, such as a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. One or more of these modifications can be used to alter selectivity of the inhibitor (e.g., relative to one specific Cystine-knot family proteins), reduce antigeinty of the TGF derepressor, alter other binding characteristics with respect to Cystine-knot proteins (such as K_{d}, and/or kₒₙ or k_{off} rates), or improve biodistribution or half life in vivo or on the shelf.

The present invention also contemplates the use of other polypeptide affinity reagents that bind to Cystine-knot family proteins and compete with the binding of a Cystine-knot binding partner, such as BMP. For instance, such affinity reagents include antibody agents, as well as peptides and scaffolded peptides that bind to and inhibit a Cystine-knot protein. Exemplary antibodies of the present invention include recombinant antibodies and monoclonal antibodies, as well as constructs derived from antigen binding fragments thereof, such as V_{H} domains, V_{L} domains, scFv's, Fab fragments, Fab′ fragments, F(ab′)₂ constructs, Fv's, and disulfide linked Fv's. In certain preferred embodiments, the antibody agent is a fully human antibody or a humanized chimeric antibody, or is an antigen binding fragment thereof.

In still other embodiments, the TGF derepressor is a small organic molecule that selectively binds to a Cystine-knot family protein and competes with the binding of a normal binding partner of Cystine-knot family proteins, such as a BMP. Preferred inhibitors of this class are molecules having molecular weights less than 2500 amu, and even more preferably less than 2000, 1000 or even 750 amu.

In certain embodiments, the TGF derepressor is selective for binding and inhibition of one specific Cystine-knot protein, as opposed to another. For instance, the TGF derepressor can be one which has a dissociation constant (K_{d}) for one Cystine-knot protein that is at least 2 times less than its K_{d} for binding another Cystine-knot protein, and even more preferably at least 5, 10, 100 or even 1000 times less. Whether by virtue of binding kinetics or biodistribution, the subject TGF derepressor can also be selected based on relative in vivo potency, such as a derepressor that has an EC₅₀ for inhibiting Cystine-knot protein activity, or a particular physiological consequence (such as promoting bone growth, or bone density increase, etc.) that is at least 2 times less than its EC₅₀ for inhibiting another Cystine-knot protein, and even more preferably at least 5, 10, 100 or even 1000 times less.

In certain preferred embodiments, the TGF derepressor binds Cystine-knot protein with a K_{d} of 1 μM or less, and more preferably a K_{d} of 100 nM, 10 nM or even 1 nM or less.

In general, the subject TGF derepressor preparations are suitable for use in a human patient. In preferred embodiments, the subject preparations of TGF derepressors will be substantially free of pyrogenic materials so as to be suitable for administration to a human patient.

In other embodiments, the subject TGF derepressors can be used to non-human animals, particularly other mammals. For example, the compounds of the present invention can be given to chickens, turkeys, livestock animals (such as sheep, pigs, horses, cattle, etc.), companion animals (e.g., cats and dogs) or may have utility in aquaculture to accelerate growth and improve the protein/fat ratio.

Another aspect of the invention relates to packaged pharmaceuticals comprising a pharmaceutical preparation of a TGF derepressor, as described herein, and a label or instructions for use in promoting growth of bone tissue in a human patient.

Still another aspect of the invention relates to packaged pharmaceuticals comprising a pharmaceutical preparation of a TGF derepressor, as described herein, and a label or instructions for veterinarian use in promoting growth of bone tissue in a non-human mammal.

Yet another aspect of the invention provides a pharmaceutical preparation suitable for use in a mammal, comprising: a vector including a coding sequence for a polypeptide TGF derepressor that binds to a BMP binding site on a Cystine-knot protein and inhibits BMP binding by the Cystine-knot protein, and transcriptional control sequences for causing expression of the polypeptide TGF derepressor in vivo in an amount effective for promoting growth of bone tissue in the treated mammal. The preparation may include agents that enhance the uptake of the vector by cells of the treated mammal.

Another aspect of the invention relates to a method for promoting BMP signal transduction in vivo by administering a pharmaceutical preparation of one or more of the subject TGF derepressors. The subject method can be used to promote all BMP-mediated biological effects, such as bone growth in human patients or in non-human animals.

In certain embodiments, the treatment methods of the present invention can be used to reduce the severity of a pathologic condition, which is characterized, at least in part, by an abnormal amount, development or metabolic activity of bone tissue in a subject. For instance, the pharmaceutical preparations of the present invention can be administered in an amount effective to prevent, ameliorate or reduce the severity of reduced bone density since diseases such as osteoporosis, or any other condition resulting from lack of BMP activity.

The present invention also contemplates the use of the subject TGF derepressor formulations conjointly with one or more other compounds useful in an effort to treat the diseases or therapeutic indications enumerated above. In these combinations, the therapeutic agents and the TGF derepressors of this invention may be independently and sequentially administered or co-administered. Combined therapy to inhibit bone resorption, prevent osteoporosis, reduce skeletal fracture, enhance the healing of bone fractures, stimulate bone formation and increase bone mineral density can be effectuated by combinations of bisphosphonates and the TGF derepressors of this invention. Bisphosphonates with these utilities include but are not limited to alendronate, tiludronate, dimethyl-APD, risedronate, etidronate, YM-175, clodronate, pamidronate, and BM-210995 (ibandronate).

The subject TGF derepressors may be combined with a mammalian estrogen agonist/antagonist. The term estrogen agonist/antagonist refers to compounds which bind with the estrogen receptor, inhibit bone turnover and prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. A variety of these compounds are described and referenced below, however, other estrogen agonists/antagonists will be known to those skilled in the art. Exemplary estrogen agonist/antagonists include droloxifene and associated compounds (see U.S. Pat. No. 5,047,431), tamoxifen and associated compounds (see U.S. Pat. No. 4,536,516), 4-hydroxy tamoxifen (see U.S. Pat. No. 4,623,660), raloxifene and associated compounds (see 4U.S. Pat. No. 4,418,068), and idoxifene and associated compounds (see U.S. Pat. No. 4,839,155).

### BRIEF DESCRIPTION OF THE DRAWINGS

FIG. 1. Known and predicted structure of proteins with an eight-membered-ring cystine-knot. Based on BMP-7 structure (14), the fold prediction of the CAN family is illustrated. C1, C2, C3, C4, C5, and C6 represent the six cysteine residues that form the knot structure. G represents glycine residue, X represents any residue that is not cysteine or glycine. C′ represents additional cysteine residues that exist in the cystine-knot domain but do not participate in basic knot formation, S:S represents a disulfide bond. A) BMP-7 cystine knot structure. B) Model for the CAN subfamily of BMP antagonists.

FIG. 2. Phylogenetic relationship of BMP antagonists. Phylogenetic tree of human BMP antagonists based on the alignment of cystine-knot sequences of representative members from each subfamily. Chordin has four cystine-knot domains all of which gave the same result—the fourth cystine-knot domain for chordin is used here. The MultAlin server at http://prodes.toulouse.inra.fr/multalin/multalin.html was used for phylogenetic tree construction (66).

FIG. 3. Sequence alignment, genomic organization, and phylogenetic relationship of the eight-membered-ring subfamily of BMP antagonists. A) Sequence alignment of the CAN family members. The BCM search launcher at http://searchlauncher.bcm.tmc.edu/multi-align/multi-align.html was used for multiple sequence alignment. The six cysteine residues that form the knot are shown as white letters on a black background and are labeled as 1 to 6. The extra cysteine residues (labeled as C′ and Cx) are shown on a dark gray background, and the conserved G residue between C2 and C3 is shown on a light gray background. The newly identified BMP antagonists were underlined. B) Genomic organization of the CAN subfamily of BMP antagonists. Genomic and mRNA sequences of each gene were compared using the SPIDEY tool of NCBI at http://www.ncbi.nlm.nih.gov/IEB/Research/Ostell/Spidey/index.html in order to predict the exon-intron junctions. Each box represents an exon and each line an intron. The number on top of each exon and intron is the size in base pairs. The checkerboard pattern represents the cystine-knot location.

FIG. 4. The full-length BMP-6 sequence, and the regions (high-lighted) of BMP-6 for targeted mutagenesis in order to select mutations that do not bind at least one of BMP type I and type II receptors, but still bind Cys knot repressors of the BMPs. Selected mutations in each of regions 1 through 3 are shown at bottom. The mutations are selected so as to create glycosylation sites.

FIG. 5. The full-length BMP-5 sequence, and the regions (high-lighted) of BMP-5 for targeted mutagenesis in order to select mutations that do not bind at least one of BMP type I and type II receptors, but still bind Cys knot repressors of the BMPs. Selected mutations in each of regions 1 through 3 are shown at bottom. The mutations are selected so as to create glycosylation sites.

FIG. 6. Alignment of various TGF-beta superfamily member proteins. Regions one through three that may be altered to generate derepressor proteins are shown in asterisks at the top of the alignment.

### DETAILED DESCRIPTION OF THE INVENTION

### I. Overview

The instant invention is partly based on the finding that the Cystine-knot family of proteins, especially the 8 member ring DAN subfamily Cystine-knot proteins (such as Sclerostin), bind and antagonize the function of certain BMP (Bone Morphogenesis Protein) family proteins that have bone/cartilage morphogenesis activity. Typical members of such BMP proteins are defined below. In patients in need of the activity of these BMP proteins, such as those suffering from conditions characterized by excessive loss of bone density, e.g. osteoporosis, especially osteoporosis seen in post menopausal women, administration of at least one of the BMPs would be beneficial for the treatment of such conditions. However, BMPs are known to have other activities unrelated to bone morphogenesis. For example, BMP-2 was known to affect neural differentiation (White et al., Neuron 29: 57-71, 2001) and periarticular ossification (Reddi Nature Biotech. 16: 247-252, 1998), and to induce apoptosis in certain cells (Hallahan, Nature Med. 9: 1033-1038, 2003); BMP-6 is associated with psoriasis (Blessing et al, J. Cell Biol. 135: 227-239, 1996); TGF-βs are associated with scar and fibrosis (Lawrence, Eur. Cytokine Network 7: 363-374, 1996). Thus, while systemic administration of BMPs or its analogs/agonists might be helpful for the treatment of bone loss, some undesirable side effects may result. On the other hand, the Cystine-knot proteins such as Sclerostin are natural antagonists of BMPs. It has been demonstrated that loss of Sclerostin protein will lead to bone density increase—a desirable condition in patients in need of bone density increase.

Thus, one may administer to these patients a BMP analog, which ideally has no BMP activity, yet is capable of binding/antagonizing Cystine-knot proteins such as sclerostin. Without BMP activity, the BMP analog would not induce undesired side-effects in the patient. At the meantime, the BMP analog can bind and antagonize the inhibitory function of the Cystine-knot proteins towards BMPs, thereby de-repressing the endogenous BMP activity, leading to increased bone density and desirable increase in BMP activity in those patients. Thus the BMP analogs of the invention are also called TGF derepressors.

The Cystine-knot family of proteins, which includes Sclerostin, dan, cerberus, gremlin, and caronte, is a relatively large family of secreted glycoproteins containing a cystine knot motif (residues 80-167) at the C-terminus. These proteins share a remarkably similar 3-dimensional fold as their targets, the BMP proteins. Different Cystine-knot proteins have slightly different inhibition spectrum against the TGF-beta superfamily proteins. In this regard, each TGF derepressor of the invention may selectively bind and derepress the function of several Cystine-knot proteins. These TGF derepressors ideally are incapable of signaling through the wild-type TGF-beta receptors themselves, but are capable of binding/antagonizing the Cystine-knot repressors of the TGF-beta superfamily proteins.

In one embodiment, the TGF derepressors of the invention are BMP analogs with mutations that substantially reduce binding affinity of BMPs for type I receptors, but maintain substantially the same overall binding affinity towards the Cystine-knot family proteins.

The crystal structure of BMP-7 in complex with the Cystine-knot family protein Noggin was resolved by Groppe et al. (Nature 420(6916): 636-42, 2002). The structure shows that Noggin inhibits BMP signalling by blocking the molecular interfaces of the binding epitopes for both type I and type II receptors, and confirming that Noggin functions by sequestering its ligand in an inactive complex. Key residues on BMP-7 that interact with Noggin were identified in this study. On the other hand, Kirsch et al. (EMBO J. 19(13): 3314-24, 2000) characterized binding epitopes of BMP-2 for BMPR-IA (type I) and BMPR-II or ActR-II (type II) using a host of BMP-2 mutant proteins. A large epitope 1 for high-affinity BMPR-IA binding was detected spanning the interface of the BMP-2 dimer. A smaller epitope 2 for the low-affinity binding of BMPR-II was found to be assembled by determinants of a single monomer. Symmetry-related pairs of the two juxtaposed epitopes occur near the BMP-2 poles. Mutations in both epitopes yielded BMP-2 variants with reduced biological activity. Many key residues involved in BMP-BMP receptor (both type-I and type-II) interaction are identified in this study.

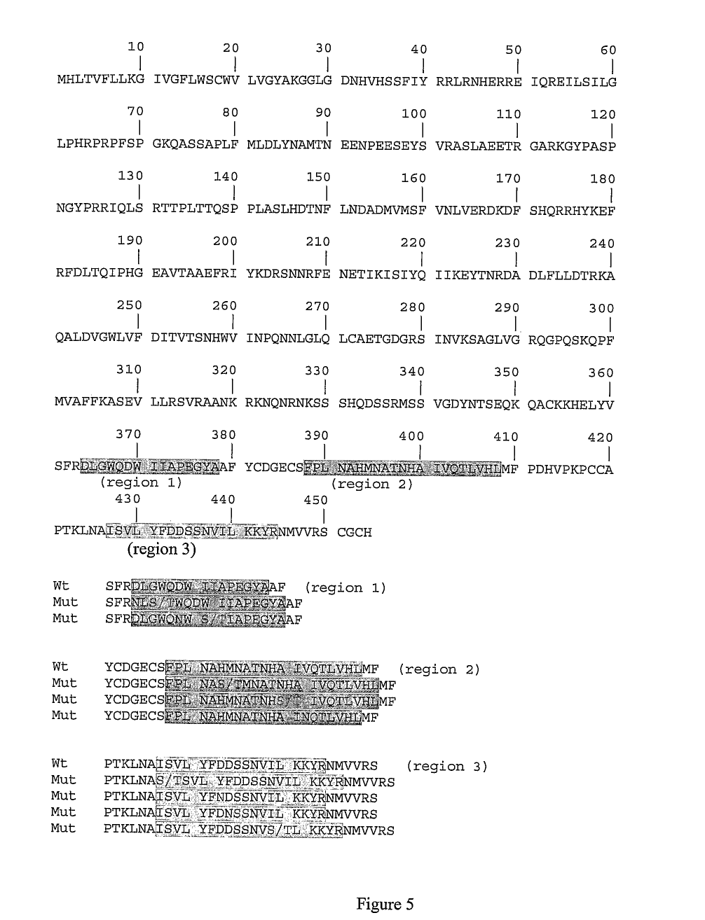

Given the sequence homology among the BMP family proteins, these findings provide a basic framework for the molecular description of receptor/antagonist recognition and BMP activation/inactivation in the BMP/TGF-beta superfamily. For example, using any of many standard molecular biology tools (such as DNAStar's MegaAlign), it is possible to align any BMP family member with BMP-2 or -7 to identify residues corresponding to those of BMP-2 and -7, which are important for receptor binding or repressor binding. Additional or independent verification can be obtained by any of many standard molecular modeling tools. For example, as illustrated in the section below, three-dimensional structures of closely related proteins can be accurately predicted based on sequence homology (see FIG. 5), using standard tools such as the three-dimensional fold recognition server at http://www.sbg.bio.ic.ac.uk/˜3dpssm/(64) and the ICM Browser (Molsoft L.L.C.) http://www.molsoft.com/products/icm_browser.htm. Based on that information, residues that are important for receptor binding but not important for binding to the Cystine-knot proteins can be selectively mutated to generate candidate TGF derepressors. In vitro and/or in vivo assays, such as in vitro binding assay for BMP receptors and Cystine-knot repressors can be used to verify the reduced/eliminated BMP receptor binding, and unchanged/enhanced Cystine-knot repressor binding.

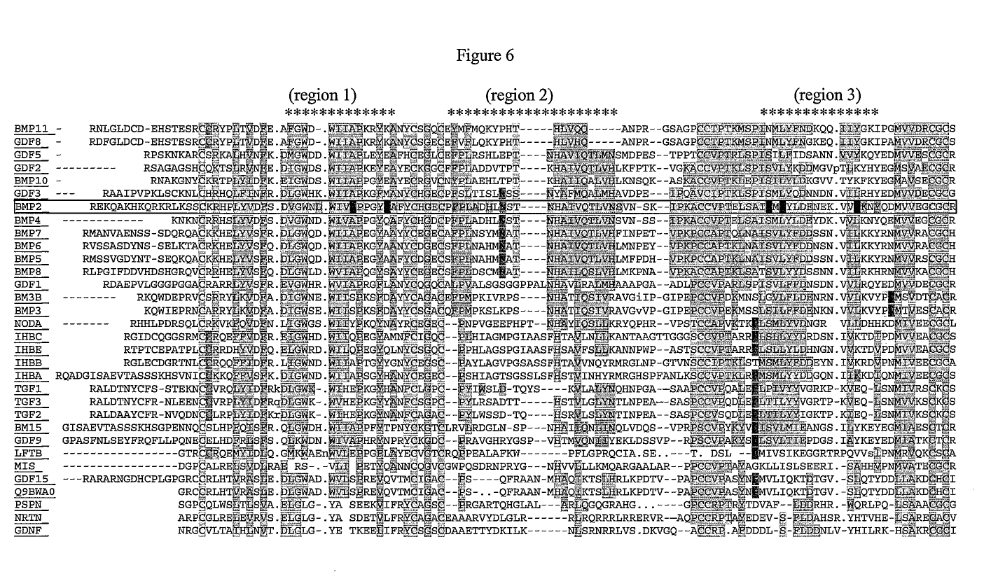

To illustrate, FIGS. 6 and 7 highlights the regions of the BMPs that can be targeted for scanning mutagenesis to identify such TGF derepressors with diminished BMP receptor binding, but substantially unaffected Cystine-knot protein binding.

Alternatively, the TGF derepressors of the invention can be generated by random mutagenesis, coupled with any of many art-recognized selective binding screenings (such as phage display, two-hybrid assay, etc.). Briefly, random mutagenesis of a target BMP protein may be carried out using any of the art-recognized the methods. The pool of mutants can then be screened for mutants that have retained their ability to bind Cystine-knot repressors, but have lost or substantially reduced ability to bind BMP receptors. Further in vitro and/or in vivo assays may be employed to verify that the selected TGF derepressors have largely lost the ability to signal through the BMP receptors. Compared to the targeted mutagenesis approach described above, random mutatgenesis is advantageous in that in may identify mutations outside the regions purportedly important for direct BMP-receptor or -repressor binding. This unbiased approach may also be automated or semi-automated in high throughput screening.

Regardless of how the TGF derepressor is obtained, in a preferred embodiment, more than one BMP mutations may be introduced to synergistically reduce BMP receptor binding, and ideally simultaneously enhance Cystine-knot repressor binding.

In another preferred embodiment, the TGF derepressor has at least about 2-, 3-, 5-, 10-, 20-, 50-, 100-, 200-, 500-, 1000-fold or more reduction of binding to BMP type I receptor, or type II receptor, or both.

In one preferred embodiment, the TGF derepressor has no more than 50%, 30%, 20%, or 10% reduction in Cystine-knot repressor binding. Most preferably, the TGF derepressor has at least about 20%, 40%, 50%, 75%, 100%, 2-fold, 5-fold, 10-fold, 50-fold, 100-fold or more increase in Cystine-knot repressor binding.

In one embodiment, the reduced binding to the BMP receptors are achieved by reduced kₒₙ alone, or increased k_{off} alone, or a combination of both.

In one embodiment, the increased binding to the Cystine-knot repressors are achieved by increased kₒₙ alone, or decreased k_{off} alone, or a combination of both.

Surface plasmon resonance could be used to measure these kinetic parameters.

In another embodiment, the subject TGF derepressors are BMP mutants that have lost their ability to form homodimers. Such monomers may still largely retain their binding affinity to the Cystine-knot repressors, but are unable to signal through the wild-type BMP receptors themselves.

The present invention also contemplates the use of the subject TGF derepressor formulations conjointly with one or more other compounds useful in an effort to treat the diseases or therapeutic indications enumerated herein. In these combinations, the therapeutic agents and the TGF derepressors of the instant invention may be independently and sequentially administered or co-administered. Combined therapy to inhibit bone resorption, prevent osteoporosis, reduce skeletal fracture, enhance the healing of bone fractures, stimulate bone formation and increase bone mineral density can be effectuated by combinations of bisphosphonates and the TGF derepressors of this invention. Bisphosphonates with these utilities include but are not limited to alendronate, tiludronate, dimethyl-APD, risedronate, etidronate, YM-175, clodronate, pamidronate, and BM-210995 (ibandronate).

The subject TGF derepressors may also be combined with a mammalian estrogen agonist/antagonist. The term estrogen agonist/antagonist refers to compounds which bind with the estrogen receptor, inhibit bone turnover and prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. A variety of these compounds are described and referenced below, however, other estrogen agonists/antagonists will be known to those skilled in the art. Exemplary estrogen agonist/antagonists include droloxifene and associated compounds (see U.S. Pat. No. 5,047,431), tamoxifen and associated compounds (see U.S. Pat. No. 4,536,516), 4-hydroxy tamoxifen (see U.S. Pat. No. 4,623,660), raloxifene and associated compounds (see 4U.S. Pat. No. 4,418,068), and idoxifene and associated compounds (see U.S. Pat. No. 4,839,155).

### II. Definitions

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. The scope an meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

“BMP” or “bone morphogenetic protein” as used herein includes the BMP subfamily of TGF-β superfamily proteins. More than 30 BMPs have been identified to date (for review, see Ducy and Karsenty, Kidney Int. 2000 June; 57(6):2207-14). Individually, the members of this subfamily of secreted molecules are termed either BMPs, osteogenic proteins (OPs), cartilage-derived morphogenetic proteins (CDMPs), or growth and differentiation factors (GDFs). They have been classified into several subgroups according to their structural similarities. BMP at least includes, but are not limited to: BMP-2 (−2A), -3, -3B (GDF-10), -4 (−2B), -5, -6, -7 (OP-1), -8 (OP-2), -9 (GDF-2), -10, -11 (GDF-11), -12 (GDF-7), -13 (GDF-6), PC8 (OP-3), DPP, 60A, Vg1, Vgr-1, Univin, GDF-5, GDF-3, GDF-1, etc., and their homologs in various species (see FIG. 8). BMP also includes any derivatives, natural or artificial, that share at least 55%, 60%, 65%, 70%, 80%, 90%, 95%, or 99% sequence identity, or at least 65%, 70%, 75%, 80%, 85%, 90%, 95%, or 99% sequence similarity/homology to the C-terminal 102 amino acid of human OP-1.

BMP functions includes bone/cartilage formation, at least in ectopic bone formation assays (such as bone nodual formation assay), cell proliferation and differentiation; apoptosis; morphogenesis; patterning of various organs, including the skeleton; and organogenesis (Graff J M, Cell 89: 171-174, 1997; Ebendal et al., J Neurosci Res 51: 139-146, 1998; Wozney, Eur J Oral Sci 106: 160-166, 1998).

“Cystine-knot protein” or “BMP inhibitor/repressor/antagonist” of the invention, in their various grammatical forms, include the family of proteins that have the conserved C-terminal 8-, 9- or 10-membered ring structure (Cystine-knot), and inhibit the function of at least one BMP proteins described above, by binding to these BMPs and interfering with their binding to the BMP receptors (type I or II or bpth). Representative members of the Cystine-knot proteins in human and other selected animals are described in detail below.

“TGF derepressor” of the invention includes BMP analogs or mutants that have impaired or diminished ability to bind at least one type of the BMP receptors (maybe both types of receptors), while substantially retain their ability to bind Cystine-knot proteins. In certain embodiments, the TGF derepressors of the invention contain mutations in the BMP receptor binding domains that do not substantially affect their ability to bind Cystine-knot proteins.

The methods of the invention may include steps of comparing sequences to each other, including wild-type sequence to one or more mutants/sequence variants Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, or “A”) in the polymer sequence not containing the inserted or deleted residue.

The term “bone disease” refers to any bone disease, disorder or state which results in or is characterized by loss of health or integrity to bone, and includes unwanted or undesired increases and decreases in bone density, growth and/or formation. Bone disease includes, but is not limited to, osteoporosis, osteopenia, faulty bone formation or resorption, Paget's disease, fractures and broken bones, bone metastasis, osteopetrosis, osteosclerosis and osteochondrosis. In the case of drug conjugates incorporating β-adrenergic antagonists, exemplary bone diseases which can be treated and/or prevented in accordance with the present invention include bone diseases characterized by a decreased bone mass relative to that of corresponding non-diseased bone, such as osteoporosis, osteopenia and Paget's disease. Drug conjugates incorporating β-adrenergic agonists can be used to treat bone diseases characterized by an increased bone mass relative to that of corresponding non-diseased bone, and include osteopetrosis, osteosclerosis and osteochondrosis.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

The term “sequence similarity,” in all its grammatical forms, refers to the degree of identity or correspondence between nucleic acid or amino acid sequences that may or may not share a common evolutionary origin.

However, in common usage and in the instant application, the term “homologous,” when modified with an adverb such as “highly,” may refer to sequence similarity and may or may not relate to a common evolutionary origin.

A nucleic acid molecule is “hybridizable” to another nucleic acid molecule, such as a cDNA, genomic DNA, or RNA, when a single stranded form of the nucleic acid molecule can anneal to the other micleic acid molecule under the appropriate conditions of temperature and solution ionic strength (see Sambrook et al. Molecular Cloning: A Laboratory Manual, Second Edition (1989) Cold Spring Harbor Laboratory Press, Cold Spring Harbor, N.Y.). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS).

Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. SSC is 0.15 M NaCl, 0.015 M Na-citrate.

“High stringent condition” is well understood in the art to encompass conditions of hybridization which allow hybridization of structurally related, but not structurally dissimilar, nucleic acids. The term “stringent” is a term of art which is understood by the skilled artisan to describe any of a number of alternative hybridization and wash conditions which allow annealing of only highly complementary nucleic acids.

Exemplary high stringent hybridization conditions is equivalent to about 20-27° C. below the melting temperature (Tₘ) of the DNA duplex formed in about 1 M salt. Many equivalent procedures exist and several popular molecular cloning manuals describe suitable conditions for stringent hybridization and, furthermore, provide formulas for calculating the length of hybrids expected to be stable under these conditions (see e.g. Current Protocols in Molecular Biology, John Wiley & Sons, N.Y. (1989), 6.3.1 6 or 13.3.6; or pages 9.47-9.57 of Sambrook, et al. (1989) Molecular Cloning, 2nd ed., Cold Spring Harbor Press).

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm, for hybrids of nucleic acids having those sequences. The relative stability (corresponding to higher Tm) of micleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.51). For hybridization with shorter nucleic acids, i.e., oligonucleotides, the position of mismatches becomes more important, and the length of the oligonucleotide determines its specificity (see Sambrook et al., supra, 11.8). A minimum length for a hybridizable nucleic acid is at least about 10 nucleotides; preferably at least about 15 nucleotides; and more preferably the length is at least about 20 nucleotides.

Unless specified, the term “standard hybridization conditions” refers to a Tₘ of about 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC, 0.05% sodium pyrophosphate, or other conditions that afford equivalent levels of hybridization.

“Polypeptide,” “peptide” or “protein” are used interchangeably to describe a chain of amino acids that are linked together by chemical bonds called “peptide bonds.” A protein or polypeptide, including an enzyme, may be a “native” or “wild-type,” meaning that it occurs in nature; or it may be a “mutant,” “variant,” or “modified,” meaning that it has been made, altered, derived, or is in some way different or changed from a native protein or from another mutant.

The terms “antibody” and “antibody agent” are used interchangeably herein, and refer to an immunoglobulin molecule obtained by in vitro or in vivo generation of the humoral response, and includes both polyclonal and monoclonal antibodies. The term also includes genetically engineered forms such as chimeric antibodies (e.g., humanized murine antibodies), heteroconjugate antibodies (e.g., bispecific antibodies), and recombinant single chain Fv fragments (scFv). The term “antibody” also includes antigen binding forms of antibodies (e.g., Fab′, F(ab′)2, Fab, Fv, rIgG, and, inverted IgG). An antibody immunologically reactive with the ALK7 epitope can be generated in vivo or by recombinant methods such as selection of libraries of recombinant antibodies in phage or similar vectors. See, e.g., Huse et al. (1989) Science 246:1275-1281; and Ward, et al. (1989) Nature 341:544-546; and Vaughan et al. (1996) Nature Biotechnology, 14:309-314.

The term “antigen binding fragment” includes any portion of an antibody that binds to the target epitope. An antigen binding fragment may be, for example, a polypeptide including a CDR3 region, or other fragment of an immunoglobulin molecule which retains the affinity and specificity of the myostatin epitope.

“Specifically binds” includes reference to the preferential association of a ligand, in whole or part, with a particular target molecule (i.e., “binding partner” or “binding moiety”) relative to compositions lacking that target molecule. It is, of course, recognized that a certain degree of non-specific interaction may occur between the subject myostatin neutralizing antibodies and a other proteins. Nevertheless, specific binding, may be distinguished as mediated through specific recognition of the myostatin protein. Typically specific binding results in a much stronger association between the antibody and target protein than between the antibody and other proteins. Specific binding by an antibody to target under such conditions requires an antibody that is selected for its specificity for a particular protein. The affinity constant (Kₐ, as opposed to K_{d}) of the antibody binding site for its cognate monovalent antigen is at least 10⁷, usually at least 10⁸, preferably at least 10⁹, more preferably at least 10¹⁰, and most preferably at least 10¹¹ M. A variety of immunoassay formats are appropriate for selecting antibodies specifically reactive with the target. For example, solid-phase ELISA immunoassays are routinely used to select monoclonal antibodies specifically reactive with a protein. See Harlow and Lane (1988) Antibodies, A Laboratory Manual, Cold Spring Harbor Publications, New York, for a description of immunoassay formats and conditions that can be used to determine specific reactivity.

Immunoassays in the competitive binding format can be used to determine cross-reactivity of antibodies with a target, e.g., to identify whether a test antibody is a target neutralizing antibody. For example, the target protein, or an epitope thereof is immobilized to a solid support. Test antibodies are added to the assay compete with the binding of the target to the immobilized antigen. The ability of the test antibodies to compete with the binding of the target to the immobilized antigen is compared.

Similarly, immunoassays in the competitive binding format can be used to determine cross-reactivity determinations, e.g., to determine the specificity of a target neutralizing antibody. For example, the target protein, or an epitope thereof is immobilized to a solid support. Epitopes from other proteins, such as GDF11 or other proteins having sequence homology with myostatin are added to the assay to compete with the binding of a potential target neutralizing antibody to the immobilized antigen. The ability of the test peptides to compete with the binding of potential target neutralizing antibody with the immobilized antigen is compared. The percent cross-reactivity of the potential target neutralizing antibody for the other antigens is calculated, using standard calculations.

### III. Cystein-Knot Containing BMP Antagonists

Availability of completed genome sequences from diverse organisms allows bioinformatic analysis of the evolution of BMP antagonists and facilitates their classification. With the aid of a regular expression algorithm (http://BioRegEx.stanford.edu), an exhaustive search of the human genome identified all cystine knot-containing BMP antagonists. Based on the size of the cystine ring, these proteins were divided into three subfamilies: CAN (eight-membered ring), twisted gastrulation (nine-membered ring), as well as chordin and noggin (ten-membered ring). The CAN family can be divided further into four subgroups based on a conserved arrangement of additional cysteine residues—gremlin and PRDC, cerberus and coco, and DAN, together with USAG-1 and sclerostin. Orthologs of these human BMP antagonists in the genomes of several model organisms have also been identified, and their phylogenetic relationship has been analyzed. New human paralogs were identified together with the verification of orthologous relationships of known genes.

Disulfide bondings formed by pairs of cysteine residues in proteins are essential for the formation of unique functional motifs and protein folds. Four half-cystine residues (the oxidized form of cysteine), which form two intrachain disulfide bonds, represent a unique framework for the formation of a cystine ring motif originally identified in mammalian endothelin and insect derived neurotoxins (1, 2). This ring structure is conserved in diverse families of proteins including the cystine-knot superfamily (3) that is characterized by the participation of a third pair of cysteine residues. For cystine-knot proteins, the disulfide bonds are arranged so the third disulfide bond that threads back through the ring forms the characteristic knot motif (4), making the structure of these proteins exceptionally stable. Proteins with cystine knots exist in many unrelated species and it has been hypothesized that this structural fold emerged multiple times as the result of convergent evolution (5). Proteins with a cystine-knot motif are usually secreted by cells and play important roles in extracellular signaling in multicellular metazoans. In addition to the cystine-knot-containing ligands, they include ion channel blockers, haemolytic agents, as well as molecules having antiviral and antibacterial activities (4, 6).

The cystine-knot superfamily of ligands comprises many homodimeric and heterodimeric proteins that are involved in embryonic development, organogenesis, as well as tissue remodeling and repair. Included in this superfamily are the Transforming Growth Factor (TGF)-betas, Growth Differentiation Factors (GDFs), Bone Morphogenetic Proteins (BMPs), BMP antagonists, gonadotropins, and Platelet-Derived Growth Factors (PDGFs)(7). In addition to the regulation of normal cell functions, many cystine-knot-containing proteins have been implicated in tumorigenesis (8-10).

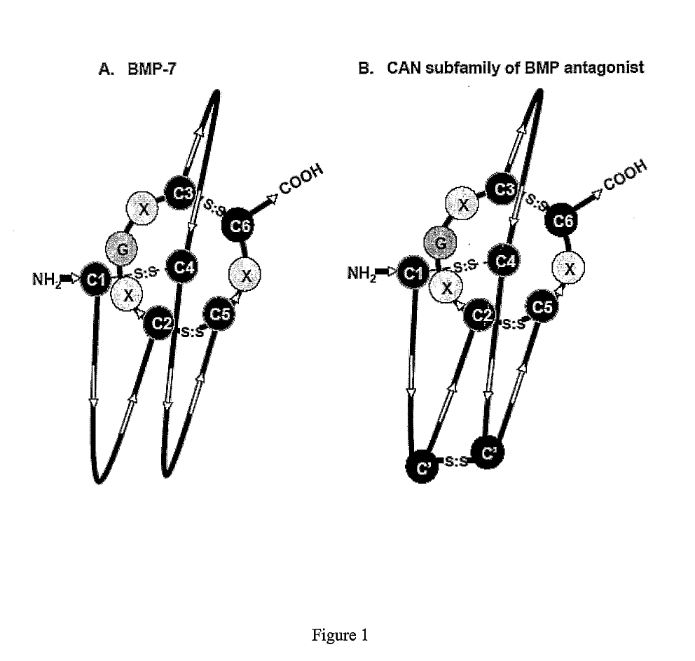

BMP proteins were first identified in the protein extracts of demineralized bone (11) and are involved in body patterning and morphogenesis (8). The developmental signaling pathway mediated by vertebrate BMPs and their fly ortholog Decapentaplegic (DPP) is highly conserved during animal evolution. This pathway is required for dorsal-ventral patterning of the early embryo in both vertebrates and invertebrates (8), and the BMP family genes have undergone expansion during evolution leading to the generation of multiple paralogs in human (8). Several BMP paralogs were named as GDFs based on their roles in the growth and differentiation of diverse tissues (8, 12, 13). Loss- or gain-of-function mutations of the vertebrate BMP and GDF genes display a variety of developmental defects (8). Among the BMP proteins, the three-dimensional structure of BMP7 has been elucidated (FIG. 1A) (14).

In addition to the tissue-specific expression of BMP ligands and their cell surface receptors, a crucial regulatory step of BMP signaling is modulation by specific BMP antagonists. Several groups of BMP antagonists were identified based on their ability to block the actions of BMP proteins through direct binding (15, 16). These antagonists include the CAN family of proteins (15, 17, 18), the twisted gastrulation protein (19, 20), the chordin family that includes chordin and ventroptin (21, 22), and noggin (23-25). Of particular interest, these BMP antagonists have a cysteine arrangement consistent with the formation of the cystine-knot structure and represent a subfamily of cystine-knot proteins. Few orthologs for the BMP antagonists are present in invertebrates and the BMP antagonist subgroup underwent major expansion during vertebrate evolution (7).

Recent availability of genome sequences from multiple vertebrates (26, 27) and the sea squirt (Ciona intestinalis) (28) provides the opportunity to perform bioinformatic searches of BMP antagonists in the GenBank based on their unique cysteine arrangements. Based on the phylogenetic relationship of different human BMP antagonists, these proteins are divided into three subfamilies based on the known or predicted size of their cystine rings, thus unifying the classification of these proteins from diverse species based on their evolutionary relationship.

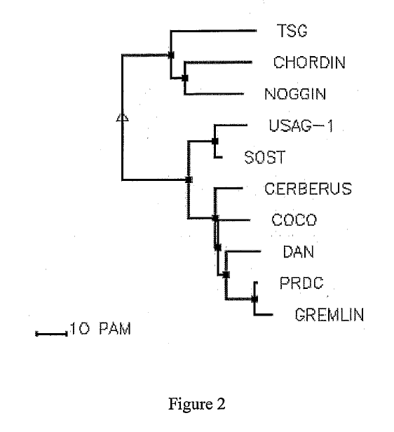

Of specific interest is the CAN family of eight-membered-ring BMP antagonists, the orthologs of which are identified in model organisms, and the three-dimensional fold of their cystine-knot motif are predicted. As shown for BMP7 (FIG. 1A) and for other eight-membered-ring cystine-knot proteins, the cysteine residues that form the cystine knot are numbered 1 to 6. Cysteine residues 2, 3, 5, and 6 form the ring and cysteine residues 1 and 4 form the knot. The cystine ring size is determined by the spacing between cysteine residues 2 and 3 (marked as C2 and C3 in FIG. 1) and cysteine residues 5 and 6 (marked as C5 and C6 in FIG. 1) (see also the gray residues in Table 1). Based on this conserved cysteine arrangement in the eight-membered-ring cystine-knot, the human proteome database for all proteins was searched with a cystine-knot motif (FIG. 2). In addition to the known proteins, hypothetical and unknown proteins that contain an eight-membered-ring cystine knot were detected and selected. All proteins with multiple cysteine residues that were not conserved in orthologs from other species were excluded. Among the hypothetical proteins, two new potential human BMP antagonists were identified. The first was found to be an ortholog of the rat USAG-1 that is preferentially expressed in the receptive rat endometrium (32), whereas the second was found to be an ortholog of the Xenopus coco gene (33). However, similar searches for nine- and ten-membered-ring cystine knot proteins did not reveal novel proteins in the human proteome (FIG. 2). All sequence data retrieved can be viewed at http://hormone.stanford.edu/BMP-antagonists (incorporated herein by reference).

Human USAG-1 maps on chromosome 7p21 and shares a 98% homology with its newly identified mouse ortholog and 97% homology with the rat USAG-1. Recently, orthologs for USAG-1 have been found in Xenopus based on functional screen for developmental genes (34). Based on EST searches, human USAG-1 is widely expressed (kidney, skin, liver, mammary gland, aorta and vein, embryonic tissues, etc.). SOST, which encodes for sclerostin, is the closest human paralog of USAG-1; these two proteins share 54% homology overall, with 64% homology in the cystine-knot domain. Coco is a new member of the CAN family of secreted BMP inhibitors recently identified in Xenopus (33). One hypothetical human protein (gi|22749329) was identified as human coco, and as in Xenopus, it is closely related to cerberus.

The searches also revealed the orthologous relationship between coco and the previously identified dante in mouse (17). The mouse dante gene represents a fragment of the predicted mouse coco, thus allowing the prediction of the full-length mouse coco protein as described below.

By aligning the sequences of all known and potential BMP antagonists, these proteins are divided into three subfamilies based on the predicted structure and spacing of the cysteine residues in the cystine rings—the eight-membered-ring, the nine-membered-ring, and two types of ten-membered-ring motifs (Table 1, gray residues).

The first subfamily is the CAN family with a cystine knot comprised of an eight-membered-ring. These proteins have a cystine knot with a ring structure similar to that of the BMPs (FIG. 1, Table 1) and glycoprotein hormone subunits (e.g. hCG-beta). The second subfamily is the twisted gastrulation protein with a carboxyl terminal cysteine arrangement that could form a cystine knot comprising a nine-membered ring (Table 1). This cystine ring does not have a glycine residue between cysteine residue numbers 2 and 3 in the first half of the ring (Cys2-X-X-X-Cys3) but has an additional residue in the second half of the ring (Cys5-X-X-Cys6). This arrangement results in a total of nine residues in the ring. The third subfamily consists of two groups of BMP antagonists with a ten-membered ring. One group is the chordin family that, in addition to chordin and its fly ortholog SOG, includes the ventroptin protein. This group has four (in the case of chordin/SOG) or three (in the case of ventroptin) cysteine-rich domains.

Analyzing the cysteine spacing and arrangement in each domain revealed a conserved arrangement (between the different domains in the same proteins and between the two human paralogs) that could form a ten-membered ring (Table 1). The first half of the ring has an additional cysteine residue (Cys2-X-Cys-X-Cys3) and the second half has two residues between cysteine residues 5 and 6 (Cys5-X-X-Cys6). The last group consists of the noggin protein with a ten-membered ring (Table 1). The three-dimensional structure of noggin is known (24).

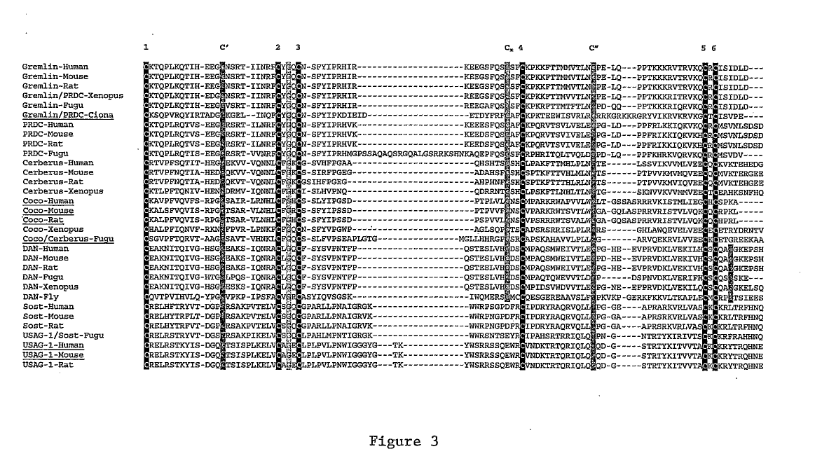

Phylogenetic analyses (35) of representative members of the three BMP antagonist subfamilies in human (FIG. 3) show that the eight-membered-ring CAN subgroups are more closely related to each other than to the nine- and ten-membered-ring subfamilies. The nine- and ten-membered-ring BMP antagonists form a second branch with the ten-membered-ring proteins (noggin and chordin) closer to each other than to the nine-membered-ring subfamily (twisted gastrulation). This phylogenetic analysis gives further support to the division of BMP antagonists into three subfamilies based on ring size in the cystine-knot motif.

### Consensus Structures of the Eight-Membered-Ring Subfamily of BMP Antagonists—the CAN Family

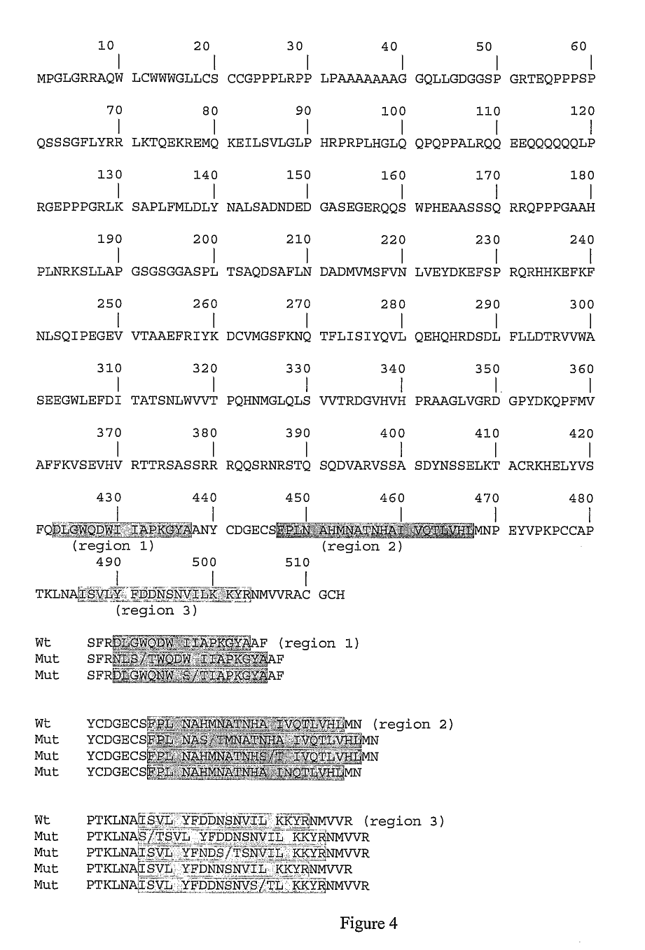

In addition to human CAN family proteins, their orthologs in diverse vertebrates were identified based on GenBank searches (FIG. 4A). All newly identified genes are underlined. Sequence comparison of all members of the human CAN family and their orthologs revealed a consensus cysteine arrangement signature (FIG. 4A). In addition to the six conserved cysteine residues that form the cystine knot, two extra cysteine residues (C′) (FIG. 4A and Table 1, dark background) are present in loop 1 and loop 2 of the cystine-knot motif, respectively. This pair of cysteine residues could form a disulfide bond.

Comparison of the cysteine residue arrangement between the CAN subfamily of BMP antagonists and known cystine-knot-containing proteins allows the prediction of cystine-knot folds for CAN family proteins (FIG. 1B) (7). Similar to BMP7, the cysteine residues that form the cystine knot are marked as 1 to 6. Cysteine residues 2, 3, 5, and 6 form the ring and cysteine residues 1 and 4 form the knot. The additional cysteine residues (marked as C′) located in the loops of the cystine knot are predicted to form an additional intra-subunit disulfide bond (FIG. 1B).

For several BMP antagonists of the CAN family, an additional cysteine residue, Cx (Table 1, FIG. 4A), is present in the heel of the cystine knot two residues upstream of the Cys4 that forms the knot with Cys1. This extra cysteine residue could allow the formation of homodimers, but is missing in sclerostin and USAG-1. In contrast, the DAN protein has another cysteine two residues downstream of the Cys6 (Table 1, FIG. 4A).

All members of the CAN subfamily of BMP antagonists can be found in man and mouse in syntenic chromosomal regions, further confirming their orthologous relationship (Table 2). All of them are conserved proteins with a cystine-knot motif in the carboxyl terminus (Table 3) (15, 17, 18, 36-40). Although the predicted proteins vary in length, they all have a signal peptide for secretion and putative N-linked glycosylation sites (Table 3). CAN family members are believed to regulate embryonic and organ development by selectively antagonizing the activities of different BMP ligands (15, 41) (Table 4). Together with USAG-1, there are a total of seven CAN family members.

Gremlin/DRM/IHG-2 Gremlin was first isolated from the neural crest of the Xenopus as an antagonist of BMP signaling (15, 42) and is an important BMP regulator for limb development that acts in a complementary fashion with other BMP antagonists (43). Gremlin binds directly to BMP2/4 and prevents them from interacting with their receptors (Table 4) (44). High levels of gremlin expression were found in nondividing and terminally differentiated cells such as neurons, alveolar epithelial cells, and goblet cells (18). Gremlin also is known as DRM (Down Regulated by v-mos) because it was identified as a gene that is downregulated in mos-transformed cells. Another name for gremlin is IHG-2 (Induced in High Glucose 2) (45) because its expression in mesangial cells, derived from glomerular messangium of the kidney, is induced by high ambient glucose, mechanical strain, and TGF-beta. Gremlin was suggested as a modulator of mesangial cell proliferation and epithelial-mesenchymal transdifferentiation in a diabetic milieu (46). Increased expression of gremlin has recently been demonstrated in several models of diabetic nephropathy. Gremlin is involved in the pathophysiology of glomerulosclerosis and tubulointerstitial fibrosis, and represents a potential therapeutic target in progressive renal diseases (47).

```
Human Gremlin/DRM/IHG-2 protein (NCBI RefSeq ID: NP_037504):
      1 msrtaytvga lllllgtllp aaegkkkgsq gaipppdkaq hndseqtqsp qqpgsrnrgr
    61 gqgrgtampg eevlessqea lhvterkylk rdwcktqplk qtiheegcns rtiinrfcyg
  121 qcnsfyiprh irkeegsfqs csfckpkkft tmmvtlncpe lqpptkkkrv trvkqcrcis
  181 idld
Human Gremlin/DRM/IHG-2 cDNA (NCBI RefSeq ID: NM_013372):
      1 gcggccgcac tcagcgccac gcgtcgaaag cgcaggcccc gaggacccgc cgcactgaca
    61 gtatgagccg cacagcctac acggtgggag ccctgcttct cctcttgggg accctgctgc
  121 cggctgctga agggaaaaag aaagggtccc aaggtgccat ccccccgcca gacaaggccc
  181 agcacaatga ctcagagcag actcagtcgc cccagcagcc tggctccagg aaccgggggc
  241 ggggccaagg gcggggcact gccatgcccg gggaggaggt gctggagtcc agccaagagg
  301 ccctgcatgt gacggagcgc aaatacctga agcgagactg gtgcaaaacc cagccgctta
  361 agcagaccat ccacgaggaa ggctgcaaca gtcgcaccat catcaaccgc ttctgttacg
  421 gccagtgcaa ctctttctac atccccaggc acatccggaa ggaggaaggt tcctttcagt
  481 cctgctcctt ctgcaagccc aagaaattca ctaccatgat ggtcacactc aactgccctg
  541 aactacagcc acctaccaag aagaagagag tcacacgtgt gaagcagtgt cgttgcatat
  601 ccatcgattt ggattaagcc aaatccaggt gcacccagca tgtcctagga atgcagcccc
  661 aggaagtccc agacctaaaa caaccagatt cttacttggc ttaaacctag aggccagaag
  721 aacccccagc tgcctcctgg caggagcctg cttgtgcgta gttcgtgtgc atgagtgtgg
  781 atgggtgcct gtgggtgttt ttagacacca gagaaaacac agtctctgct agagagcact
  841 ccctattttg taaacatatc tgctttaatg gggatgtacc agaaacccac ctcaccccgg
  901 ctcacatcta aaggggcggg gccgtggtct ggttctgact ttgtgttttt gtgccctcct
  961 ggggaccaga atctcctttc ggaatgaatg ttcatggaag aggctcctct gagggcaaga
1021 gacctgtttt agtgctgcat tcgacatgga aaagtccttt taacctgtgc ttgcatcctc
1081 ctttcctcct cctcctcaca atccatctct tcttaagttg atagtgacta tgtcagtcta
1141 atctcttgtt tgccaaggtt cctaaattaa ttcacttaac catgatgcaa atgtttttca
1201 ttttgtgaag accctccaga ctctgggaga ggctggtgtg ggcaaggaca agcaggatag
1261 tggagtgaga aagggagggt ggagggtgag gccaaatcag gtccagcaaa agtcagtagg
1321 gacattgcag aagcttgaaa ggccaatacc agaacacagg ctgatgcttc tgagaaagtc
1381 ttttcctagt atttaacaga acccaagtga acagaggaga aatgagattg ccagaaagtg
1441 attaactttg gccgttgcaa tctgctcaaa cctaacacca aactgaaaac ataaatactg
1501 accactccta tgttcggacc caagcaagtt agctaaacca aaccaactcc tctgctttgt
1561 ccctcaggtg gaaaagagag gtagtttaga actctctgca taggggtggg aattaatcaa
1621 aaacctcaga ggctgaaatt cctaatacct ttcctttatc gtggttatag tcagctcatt
1681 tccattccac tatttcccat aatgcttctg agagccacta acttgattga taaagatcct
1741 gcctctgctg agtgtacctg acagtagtct aagatgagag agtttaggga ctactctgtt
1801 ttagcaagag atattttggg ggtctttttg ttttaactat tgtcaggaga ttgggctaaa
1861 gagaagacga cgagagtaag gaaataaagg gaattgcctc tggctagaga gtagttaggt
1921 gttaatacct ggtagagatg taagggatat gacctccctt tctttatgtg ctcactgagg
1981 atctgagggg accctgttag gagagcatag catcatgatg tattagctgt tcatctgcta
2041 ctggttggat ggacataact attgtaacta ttcagtattt actggtaggc actgtcctct
2101 gattaaactt ggcctactgg caatggctac ttaggattga tctaagggcc aaagtgcagg
2161 gtgggtgaac tttattgtac tttggatttg gttaacctgt tttcttcaag cctgaggttt
2221 tatatacaaa ctccctgaat actctttttg ccttgtatct tctcagcctc ctagccaagt
2281 cctatgtaat atggaaaaca aacactgcag acttgagatt cagttgccga tcaaggctct
2341 ggcattcaga gaacccttgc aactcgagaa gctgttttta tttcgttttt gttttgatcc
2401 agtgctctcc catctaacaa ctaaacagga gccatttcaa ggcgggagat attttaaaca
2461 cccaaaatgt tgggtctgat tttcaaactt ttaaactcac tactgatgat tctcacgcta
2521 ggcgaatttg tccaaacaca tagtgtgtgt gttttgtata cactgtatga acacaccaca
2581 aatctttgta ttgtccacat tctccaacaa taaagcacag agtggattta attaagcaca
2641 caaatgctaa ggcagaattt tgagggtggg agagaagaaa agggaaagaa gctgaaaatg
2701 taaaaccaca ccagggagga aaaatgacat tcagaaccag caaacactga atttctcttg
2761 ttgttttaac tctgccacaa gaatgcaatt tcgttaacgg agatgactta agttggcagc
2821 agtaatcttc ttttaggagc ttgtaccaca gtcttgcaca taagtgcaga tttggctcaa
2881 gtaaagagaa tttcctcaac actaacttca ctgggataat cagcagcgta actaccctaa
2941 aagcatatca ctagccaaag agggaaatat ctgttcttct tactgtgcct atattaagac
3001 tagtacaaat gtggtgtgtc ttccaacttt cattgaaaat gccatatcta taccatattt
3061 tattcgagtc actgatgatg taatgatata ttttttcatt attatagtag aatattttta
3121 tggcaagata tttgtggtct tgatcatacc tattaaaata atgccaaaca ccaaatatga
3181 attttatgat gtacactttg tgcttggcat taaaagaaaa aaacacacat cctggaagtc
3241 tgtaagttgt tttttgttac tgtaggtctt caaagttaag agtgtaagtg aaaaatctgg
3301 aggagaggat aatttccact gtgtggaatg tgaatagtta aatgaaaagt tatggttatt
3361 taatgtaatt attacttcaa atcctttggt cactgtgatt tcaagcatgt tttctttttc
3421 tcctttatat gactttctct gagttgggca aagaagaagc tgacacaccg tatgttgtta
3481 gagtctttta tctggtcagg ggaaacaaaa tcttgaccca gctgaacatg tcttcctgag
3541 tcagtgcctg aatctttatt ttttaaattg aatgttcctt aaaggttaac atttctaaag
3601 caatattaag aaagacttta aatgttattt tggaagactt acgatgcatg tatacaaacg
3661 aatagcagat aatgatgact agttcacaca taaagtcctt ttaaggagaa aatctaaaat
3721 gaaaagtgga taaacagaac atttataagt gatcagttaa tgcctaagag tgaaagtagt
3781 tctattgaca ttcctcaaga tatttaatat caactgcatt atgtattatg tctgcttaaa
3841 tcatttaaaa acggcaaaga attatataga ctatgaggta ccttgctgtg taggaggatg
3901 aaaggggagt tgatagtctc ataaaactaa tttggcttca agtttcatga atctgtaact
3961 agaatttaat tttcacccca ataatgttct atatagcctt tgctaaagag caactaataa
4021 attaaaccta ttctttcaaa aaaaaa
```

PRDC Protein Related to DAN and Cerberus (PRDC) was first described in mouse (48) and shares high homology with gremlin.

```
Human PRDC protein (NCBI RefSeq ID: NP_071914):
      1 mfwklslslf mvavlvkvae arknrpagai hspykdgssn nserwqhqik evlassqeal
    61 vvterkylks dwcktqplrq tvseegcrsr tilnrfcygq cnsfyiprhv kkeeesfqsc
  121 afckpqrvts vlvelecpgl dppfrlkkiq kvkqcrcmsv nlsdsdkq
Human PRDC cDNA (NCBI RefSeq ID: NM_022469):
      1 agcgggctct cgcctctcct gcaccctcag ccggcgcgct tctcttatgg gcgtctgctg
    61 cagtctggct gcggtcgaac tgaaagcggc ggcgggagac caaacttaga ccccgctgtg
  121 gactagagaa ctcagagaag gcagagggag agggagagag agagagagaa gggacccgag
  181 gaggaggctt ccatcacgtc attgcaggat gttctggaag ctttccctgt ccttgttcat
  241 ggtggcggtg ctggtgaagg tggcggaagc ccggaagaac cggccggcgg gcgccatcca
  301 ctcgccttac aaggacggca gcagcaacaa atcggagaga tggcagcacc agatcaagga
  361 ggtactggcc tccagccagg aggccctggt ggtcaccgag cgcaagtacc tcaagagtga
  421 ctggtgcaag acgcagccgc tgcggcagac ggtgagcgag gagggctgcc ggagccgcac
  481 catcctcaac cgcttctgct acggccagtg caactccttc tacatcccgc ggcacgtgaa
  541 gaaggaggag gagtccttcc agtcctgcgc cttctgcaag ccccagcgcg tcacctccgt
  601 cctcgtggag ctcgagtgcc ccggcctgga cccacccttc cgactcaaga aaatccagaa
  661 ggtgaagcag tgccggtgca tgtccgtgaa cctgagcgac tcggacaagc agtgagcgcc
  721 gggcaggacg cagctcagcc ccgcgcgcgc ccggcagctg ggtggcgccg ccgccgcctc
  781 tgtccctgcc ctccgagccc actgtcacgc tgcctggtgt tccccatcag cagcaagcac
  841 ttctcttagg gctgacggtg tccttgtcac agacgtggat cgcaagtgga gcttttgctg
  901 atgtgttcct gaccgacccc gggtcccacc tgtgccccta aaccaggcgg tggatcaggc
  961 ccgaaggaaa atgctggaaa accaccaccg ccaccgagca gtcagagagg acctttcgag
1021 gtgatgcact gtaaatccgg cattgatgtt tccgccatgt ggaaaagcaa ggatttccct
1081 ggccccgccc tgcactccag ctcgcccctt tcccgcgcga agtgaaggat gcgtcacttt
1141 tctgagcggc ccctcagacc tctcctgctg gtccctggac ctcttgctgg gagcgatcat
1201 tcttgtgact ggcaggctgc cctcggtcgc tgctgtgatg aggatatgtg caacctccat
1261 aaatatgtcg gtggacgggg cgtctcctaa taaacctgat accaagaaga caacttgagc
1321 cacttggatc ccgaagtggg cttcccggga aacctcgagc agagcaggct acacaatccc
1381 ccatcctcaa gtccccaccg cctcttcttc ctcactgtcc ccgcccccta tttgtggact
1441 aaagatgaac tctggtgtgc atgctattat tgctgcaatt agaatattat cacacacaaa
1501 ggtctctata ttaacgctgg ttttataagt gaccgtatat tgtaaagagc tgttaaaaag
1561 tattatagac ctattcatgt attatttcac atgttttgac tctggcttgc agcaccattc
1621 ggagtaagga tgacagcagg cccagaaggt gttttactca gaaaaaaaaa tgcctggtca
1681 gcaattcact gtcatgcacc tatttttaga ttgggcaggg agatgggagg agtcgtgatt
1741 tttactttga atactatttc actgacgtta ctaagtattg cagcacaatg tagaaattgg
1801 cttgggatgg ataggtatag ggaagggcat tcgttgggaa attgatgacc ggaaactaga
1861 aagcccctat aatgtggaat atgcatatgt ggggtgaaac cgtatactac tttcactgcc
1921 atgacactag gcaaaaaata tttcctgtta aaagaaagag aaaaagaaag ggatattgaa
1981 aaatataagg agttgagtcg ctttgtggta tagaagggag taaggaataa aagatataga
2041 atagtaatct agttgaactg ttctgcacaa aacatgcttc cttttcaaag aatagacttc
2101 aaaagggacg aacaaacatt aggtcacctt ttgtgtttat aatgaattgt tgacgtttcc
2161 atgctgtggt ctcttgatat acggaagtag acgtaactta ttttccagcg aaattatata
2221 cttcttctgg gctcctttgc tcagttgccc caggaaggct tccctgtgag taatgcttat
2281 ggaatctgac aaatgtgaat gagctgccat tggtgggaat cagtaaactt tgaaaacctt
2341 tcactgatga ttccctggta agtgctcctg tgaggggcca cttccctagt atgtgagaga
2401 gaccctctgc aagccgctgt ggggacttag cttcctgata ctaggtagtg atggtggatc
2461 tagagacatt cacttgaaga gcaaggattg gggtggggtg ggttggtgaa tctatctcag
2521 gccactgcac tgcaaggtgc atttctgtca tttctgcaac accatgagac ctgagcaaga
2581 atgtgctgtg gcagaggggg tcttggtttc ataaatgggt atatacagac tgaactcagg
2641 aggcagaaat caaaagaaat ccttgagggc tggaacctga ggatagagcg aaaatggaga
2701 gaagggtgtt caaggtcagg agtgattttt gcatttggtg gaaaacctca agtcagactc
2761 agtggcagaa catgcagcag aaaagctgtg aaggaaggaa atttacgtga ttgtaatgga
2821 ggctaggggt ggattgggag gtgagtggag gagagtctct gaccagctgc aaagccaacc
2881 cctatcattt tattaacatg gaacccacac agatagggga gggattaagc taagaactta
2941 cagaatgcaa acacgagcac actctcttcg aacccaattg tgggtgtagc aatgaaagca
3001 atatgatatg ctgcagtgtg aagctccttc tggggttatc gtatgtacaa agtttacctt
3061 ataatggctc aaattgtatt taattttttt gttttgttta taaatgaaga aaagctataa
3121 gtataatgta attattttat aggtatacta ttaaattata gaacaaataa agataatcta
3181 ggattatatg cgaaaaaaaa aaaaaaaa
```

Cerberus This gene is expressed in the anterior endomesoderm (38, 39, 49). Caronte, a chick ortholog, is involved in left-right asymmetry in the chick embryo (49). Cerberus functions as a multivalent growth factor antagonist in the extracellular space and inhibits signaling by BMP4, nodal, and Wnt (50). Mouse cerberus binds to BMP proteins and nodal via independent sites (39), whereas the Xenopus cerberus also binds Wnt proteins and inhibits their actions (50). Cerberus has the unique property of inducing ectopic heads in the absence of trunk structures (39). The expression of cerberus during gastrulation is activated by nodal-related signals in endoderm and by Spemann-organizer factors (51).

```
Human Cerberus protein (NCBI RefSeq ID: NP_005445):
      1 mhlllfqllv llplgkttrh qdgrqnqssl spvllprnqr elptgnheea eekpdlfvav
    61 phlvatspag egqrqrekml srfgrfwkkp eremhpsrds dsepfppgtq sliqpidgmk
  121 meksplreea kkfwhhfmfr ktpasqgvil pikshevhwe tcrtvpfsqt ithegcekvv
  181 vqnnlcfgkc gsvhfpgaaq hshtscshcl pakfttmhlp lnctelssvi kvvmlveecq
  241 ckvktehedg hilhagsqds fipgvsa
Human Cerberus cDNA (NCBI RefSeq ID: NM_005454):
      1 atgcatctcc tcttatttca gctgctggta ctcctgcctc taggaaagac cacacggcac
    61 caggatggcc gccagaatca gagttctctt tcccccgtac tcctgccaag gaatcaaaga
  121 gagcttccca caggcaacca tgaggaagct gaggagaagc cagatctgtt tgtcgcagtg
  181 ccacaccttg tagccaccag ccctgcaggg gaaggccaga ggcagagaga gaagatgctg
  241 tccagatttg gcaggttctg gaagaagcct gagagagaaa tgcatccatc cagggactca
  301 gatagtgagc ccttcccacc tgggacccag tccctcatcc agccgataga tggaatgaaa
  361 atggagaaat ctcctcttcg ggaagaagcc aagaaattct ggcaccactt catgttcaga
  421 aaaactccgg cttctcaggg ggtcatcttg cccatcaaaa gccatgaagt acattgggag
  481 acctgcagga cagtgccctt cagccagact ataacccacg aaggctgtga aaaagtagtt
  541 gttcagaaca acctttgctt tgggaaatgc gggtctgttc attttcctgg agccgcgcag
  601 cactcccata cctcctgctc tcactgtttg cctgccaagt tcaccacgat gcacttgcca
  661 ctgaactgca ctgaactttc ctccgtgatc aaggtggtga tgctggtgga ggagtgccag
  721 tgcaaggtga agacggagca tgaagatgga cacatcctac atgctggctc ccaggattcc
  781 tttatcccag gagtttcagc ttga
```

Coco Studies in Xenopus indicated that coco functions as a blocker of BMP and TGF-beta signals in the ectoderm and regulates cell fate specification and competence prior to the onset of neural induction. Coco can also induce ectopic head-like structures in the neurula staged embryos (33). Coco is expressed maternally in an animal to vegetal gradient, and its expression level declines rapidly following gastrulation. In contrast to other known BMP inhibitors, coco is broadly expressed in the ectoderm until the end of the gastrulation stage (33).

```
Human Coco protein (NCBI RefSeq ID: NP_689867):
      1 mllgqlstll cllsgalptg sgrpepqspr pqswaaanqt walgpgalpp lvpasalgsw
    61 kaflglqkar qlgmgrlqrg qdevaavtlp lnpqeviqgm ckavpfvqvf srpgcsairl
  121 rnhlcfghcs slyipgsdpt plvlcnscmp arkrwapvvl wcltgssasr rrvkistmli
  181 egchcspka
Human Coco cDNA (NCBI RefSeq ID: NM_152654):
      1 agtccggaca gacagacagg cagacagacg cacggacaag cagatgctcc ttggccagct
    61 atccactctt ctgtgcctgc ttagcggggc cctgcctaca ggctcaggga ggcctgaacc
  121 ccagtctcct cgacctcagt cctgggctgc agccaatcag acctgggctc tgggcccagg
  181 ggccctgccc ccactggtgc cagcttctgc ccttgggagc tggaaggcct tcttgggcct
  241 gcagaaagcc aggcagctgg ggatgggcag gctgcagcgt gggcaagacg aggtggctgc
  301 tgtgactctg ccgctgaacc ctcaggaagt gatccagggg atgtgtaagg ctgtgccctt
  361 cgttcaggtg ttctcccggc ccggctgctc agccatacgc ctccgaaatc atctgtgctt
  421 tggtcattgc tcctctctct acatccctgg ctcggacccc accccactag tcctgtgcaa
  481 cagctgtatg cctgctcgca agcgttgggc acccgtggtc ctgtggtgtc tcactggcag
  541 ctcagcctcc cgtcgacggg tgaagatatc caccatgctg atcgaggggt gtcactgcag
  601 cccaaaagca tgaactgagc atcgtggatg ggtgcacgga gacacgcacc ttggagaaat
  661 gaggggagat ggaccaagaa agacgtggac ctggatgatg tactctgggt caagagacca
  721 gggatgcagg gttaggcaga caggtcccca gagtcctcac cctgctcccc agacagtaga
  781 cacagtgccc gtcctggagt tgcaccactg atagtcacag cacacaatga ttgacaactc
  841 actttttttt ttttttttga gatggagtct cgctctgtcg cccaggctgg agtgcagtgg
  901 cgcaatctca gctcactgca agctccacct cccgggttta tgccattctc ctgtctcagc
  961 ctcccgagta gctgggacta caggcacccg ccaacacgcc cggctaattt ttcgtatttt
1021 tagtaaagac agggtttcac cgtgttagcc aggatggtct ctatctcctg acctcgtgat
1081 ctgcctgcct tggccttatt attttttttt tttaaggaca gagtctctct ctgtcaccca
1141 ggctggagtg caatggcgcg atcttggctc actgtaactt ccacttgcca ggctcaagca
1201 gttctcctgc ctcagcctcc tgagtagctg ggactacagg cacccgccac catgcccagc
1261 taatttttgt atttttagta gagacagagt ttcaccatat tagcctggct ggtctcaaac
1321 tcctggcctc aggtgatctg cccacctcgg cctcccaaag tgctgggatc aaatccactg
1381 ttaatcatta ggctgaactg tctcttatag aatgaggtca aagacactcc cagttgcagg
1441 gagggtagat ggccccaccc agaccgagag acacagtgat gacctcagcc tagggacacc
1501 aaaaaaaaaa aaaaaaaaaa cccaaaccaa aaacgcaaac caaagcaggc aggcagacag
1561 ctgctggggg aaatcctggg gtccttgaga cagaggcagg accctcgtgt tcccagctgc
1621 ctcttgcctt gatagtggtg ctgtgtccct ctcagacccc ccacctgagt ctccacagag
1681 ccccacgcct ggcatggcat tccacagaaa ccataaaggt tggctgagtc c
```

DAN Differential screening-selected gene Aberrative in Neuroblastoma (DAN), also known as NO3 (40, 52, 53), is the founding member of the CAN family (15, 40). DAN interacts with GDF-5 in a frog embryo assay, suggesting that it may regulate signaling by the GDF-5/6/7 classes of BMPs (Table 4) (41). Like cerberus, DAN induces cement glands as well as markers of anterior neural tissues and endoderm in Xenopus animal cap assays, suggesting its role in the BMP signaling blockade. DAN is also expressed in the developing myotome. Overexpression of DAN in transformed cell lines suppresses the transformed phenotypes and reduces cell growth, thereby causing a retardation of the cell's entry into the S phase (15, 40, 41, 53, 54). DAN may play a regulatory role during development and cell growth. Although originally proposed as a tumor suppressor gene for human neuroblastoma (55), this possibility was subsequently excluded (56).

```
Human DAN protein (NCBI RefSeq ID: NP_005371):
      1 mlrvlvgavl pamllaappp inklalfpdk sawceaknit qivghsgcea ksiqnraclg
    61 qcfsysvpnt fpqsteslvh cdscmpaqsm weivtlecpg heevprvdkl vekilhcscq
  121 acgkepsheg lsvyvqgedg pgsqpgthph phphphpggq tpepedppga phteeegaed
Human DAN cDNA (NCBI RefSeq ID: NM_005380):
      1 cgcagcgcag cccagccgag cgtcgcgggg ccgccccccg ccctgccggc cgcctcgccg
    61 agcctcctgg ggcgcccggg cccgcgaccc ccgcacccag ctccgcagga ccggcgggcg
  121 cgcgcgggct ctggaggcca cgggcatgat gcttcgggtc ctggtggggg ctgtcctccc
  181 tgccatgcta ctggctgccc caccacccat caacaagctg gcactgttcc cagataagag
  241 tgcctggtgc gaagccaaga acatcaccca gatcgtgggc cacagcggct gtgaggccaa
  301 gtccatccag aacagggcgt gcctaggaca gtgcttcagc tacagcgtcc ccaacacctt
  361 cccacagtcc acagagtccc tggttcactg tgactcctgc atgccagccc agtccatgtg
  421 ggagattgtg acgctggagt gcccgggcca cgaggaggtg cccagggtgg acaagctggt
  481 ggagaagatc ctgcactgta gctgccaggc ctgcggcaag gagcctagtc acgaggggct
  541 gagcgtctat gtgcagggcg aggacgggcc gggatcccag cccggcaccc accctcaccc
  601 ccatccccac ccccatcctg gcgggcagac ccctgagccc gaggaccccc ctggggcccc
  661 ccacacagag gaagaggggg ctgaggactg aggccccccc aactcttcct cccctctcat
  721 ccccctgtgg aatgttgggt ctcactctct ggggaagtca ggggagaagc tgaagccccc
  781 ctttggcact ggatggactt ggcttcagac tcggacttga atgctgcccg gttgccatgg
  841 agatctgaag gggcggggtt agagccaagc tgcacaattt aatatattca agagtggggg
  901 gaggaagcag aggtcttcag ggctcttttt ttgggggggg tggtctcttc ctgtctggct
  961 tctagagatg tgcctgtggg agggggagga agttggctga gccattgagt gctgggggag
1021 gccatccaag atggcatgaa tcgggctaag gtccctgggg gtgcagatgg tactgctgag
1081 gtcccgggct tagtgtgagc atcttgccag cctcaggctt gagggagggc tgggctagaa
1141 agaccactgg cagaaacagg aggctccggc ccacaggttt ccccaaggcc tctcacccca
1201 cttcccatct ccagggaagc gtcgccccag tggcactgaa gtggccctcc ctcagcggag
1261 gggtttggga gtcaggcctg ggcaggaccc tgctgactcg tggcgcggga gctgggagcc
1321 aggctctccg ggcctttctc tggcttcctt ggcttgcctg gtgggggaag gggaggaggg
1381 gaagaaggaa agggaagagt cttccaaggc cagaaggagg gggacaaccc cccaagacca
1441 tccctgaaga cgagcatccc cctcctctcc ctgttagaaa tgttagtgcc ccgcactgtg
1501 ccccaagttc taggcccccc agaaagctgc cagagccggc cgccttctcc cctctcccag
1561 ggatgctctt tgtaaatatc ggatgggtgt gggagtgagg ggttacctcc ctcgccccaa
1621 ggttccagag gccctaggcg ggatgggctc gctgaacbtc gaggaactcc aggacgagga
1681 ggacatggga cttgcgtgga cagtcagggt tcacttgggc tctctctagc tccccaattc
1741 tgcctgcctc ctccctccca gctgcacttt aaccctagaa ggtggggacc tggggggagg
1801 gacagggcag gcgggcccat gaagaaagcc cctcgttgcc cagcactgtc tgcgtctgct
1861 cttctgtgcc cagggtggct gccagcccac tgcctcctgc ctggggtggc ctggccctcc
1921 tggctgttgc gacgcgggct tctggagctt gtcaccattg gacagtctcc ctgatggacc
1981 ctcagtcttc tcatgaataa attccttcaa cgccaaaaaa aaaaaaaaaa aaaaaaaaaa
2041 aaaaaaaaaa aaaaaa
```

SOST Sclerostin, encoded by the SOST gene, is a secreted osteoclast-derived BMP antagonist (57). It was originally identified as the gene responsible for sclerosteosis (58) and is highly conserved across vertebrate species (59). Sclerostin binds to BMP6 and BMP7 with high affinity and to BMP2 and BMP4 with a lower affinity leading to the inhibition of BMP6 and BMP7 activities (57). High levels of sclerostin expression were detected in long bones, cartilage, kidney, liver, placenta, and fetal skin in human and mouse (59). Sclerostin could play an important role in bone remodeling, and links bone resorption and apposition (57). Based on its suppressive role in bone formation, SOST could be important in the development of therapeutic strategies for osteoporosis (58). Loss of function of the SOST gene product leads to a progressive bone overgrowth disorder known as sclerosteosis, an autosomal recessive disorder (57). Several SOST sequences are listed below:

```
Human SOST protein (NCBI RefSeq ID: NP_079513):
      1 mqlplalclv cllvhtafrv vegqgwqafk ndateiipel geypepppel ennktmnrae
    61 nggrpphhpf etkdvseysc relhftryvt dgpcrsakpv telvcsgqcg parllpnaig
  121 rgkwwrpsgp dfrcipdryr aqrvqllcpg geaprarkvr lvasckckrl trfhnqselk
  181 dfgteaarpq kgrkprprar sakanqaele nay
Human SOST cDNA (NCBI RefSeq ID: NM_025237):
      1 agagcctgtg ctactggaag gtggcgtgcc ctcctctggc tggtaccatg cagctcccac
    61 tggccctgtg tctcgtctgc ctgctggtac acacagcctt ccgtgtagtg gagggccagg
  121 ggtggcaggc gttcaagaat gatgccacgg aaatcatccc cgagctcgga gagtaccccg
  181 agcctccacc ggagctggag aacaacaaga ccatgaaccg ggcggagaac ggagggcggc
  241 ctccccacca cccctttgag accaaagacg tgtccgagta cagctgccgc gagctgcact
  301 tcacccgcta cgtgaccgat gggccgtgcc gcagcgccaa gccggtcacc gagctggtgt
  361 gctccggcca gtgcggcccg gcgcgcctgc tgcccaacgc catcggccgc ggcaagtggt
  421 ggcgacctag tgggcccgac ttccgctgca tccccgaccg ctaccgcgcg cagcgcgtgc
  481 agctgctgtg tcccggtggt gaggcgccgc gcgcgcgcaa ggtgcgcctg gtggcctcgt
  541 gcaagtgcaa gcgcctcacc cgcttccaca accagtcgga gctcaaggac ttcgggaccg
  601 aggccgctcg gccgcagaag ggccggaagc cgcggccccg cgcccggagc gccaaagcca
  661 accaggccga gctggagaac gcctactaga gcccgcccgc gcccctcccc accggcgggc
  721 gccccggccc tgaacccgcg ccccacattt ctgtcctctg cgcgtggttt gattgtttat
  781 atttcattgt aaatgcctgc aacccagggc agggggctga gaccttccag gccctgagga
  841 atcccgggcg ccggcaaggc ccccctcagc ccgccagctg aggggtccca cggggcaggg
  901 gagggaattg agagtcacag acactgagcc acgcagcccc gcctctgggg ccgcctacct
  961 ttgctggtcc cacttcagag gaggcagaaa tggaagcatt ttcaccgccc tggggtttta
1021 agggagcggt gtgggagtgg gaaagtccag ggactggtta agaaagttgg ataagattcc
1081 cccttgcacc tcgctgccca tcagaaagcc tgaggcgtgc ccagagcaca agactggggg
1141 caactgtaga tgtggtttct agtcctggct ctgccactaa cttgctgtgt aaccttgaac
1201 tacacaattc tccttcggga cctcaatttc cactttgtaa aatgagggtg gaggtgggaa
1261 taggatctcg aggagactat tggcatatga ttccaaggac tccagtgcct tttgaatggg
1321 cagaggtgag agagagagag agaaagagag agaatgaatg cagttgcatt gattcagtgc
1381 caaggtcact tccagaattc agagttgtga tgctctcttc tgacagccaa agatgaaaaa
1441 caaacagaaa aaaaaaagta aagagtctat ttatggctga catatttacg gctgacaaac
1501 tcctggaaga agctatgctg cttcccagcc tggcttcccc ggatgtttgg ctacctccac
1561 ccctccatct caaagaaata acatcatcca ttggggtaga aaaggagagg gtccgagggt
1621 ggtgggaggg atagaaatca catccgcccc aacttcccaa agagcagcat ccctcccccg
1681 acccatagcc atgttttaaa gtcaccttcc gaagagaagt gaaaggttca aggacactgg
1741 ccttgcaggc ccgagggagc agccatcaca aactcacaga ccagcacatc ccttttgaga
1801 caccgccttc tgcccaccac tcacggacac atttctgcct agaaaacagc ttcttactgc
1861 tcttacatgt gatggcatat cttacactaa aagaatatta ttgggggaaa aactacaagt
1921 gctgtacata tgctgagaaa ctgcagagca taatagctgc cacccaaaaa tctttttgaa
1981 aatcatttcc agacaacctc ttactttctg tgtagttttt aattgttaaa aaaaaaaagt
2041 tttaaacaga agcacatgac atatgaaagc ctgcaggact ggtcgttttt ttggcaattc
2101 ttccacgtgg gacttgtcca caagaatgaa agtagtggtt tttaaagagt taagttacat
2161 atttattttc tcacttaagt tatttatgca aaagtttttc ttgtagagaa tgacaatgtt
2221 aatattgctt tatgaattaa cagtctgttc ttccagagtc cagagacatt gttaataaag
2281 acaatgaatc atgaccgaaa gaaaaaaaaa aaaaaaaaaa aaa
Mouse SOST protein (NCBI RefSeq ID: NP_077769):
      1 mqpslapcli cllvhaafca vegqgwqafr ndatevipgl geypepppen nqtmnraeng
    61 grpphhpyda kdvseyscre lhytrfltdg pcrsakpvte lvcsgqcgpa rllpnaigrv
  121 kwwrpngpdf rcipdryraq rvqllcpgga aprsrkvrlv asckckrltr fhnqselkdf
  181 gpetarpqkg rkprpgarga kanqaelena y
Mouse SOST cDNA (NCBI RefSeq ID: NM_024449):
      1 gactggagcc tgtgctaccg agtgccctcc tccacctggc agcatgcagc cctcactagc
    61 cccgtgcctc atctgcctac ttgtgcacgc tgccttctgt gctgtggagg gccaggggtg
  121 gcaagccttc aggaatgatg ccacagaggt catcccaggg cttggagagt accccgagcc
  181 tcctcctgag aacaaccaga ccatgaaccg ggcggagaat ggaggcagac ctccccacca
  241 tccctatgac gccaaagatg tgtccgagta cagctgccgc gagctgcact acacccgctt
  301 cctgacagac ggcccatgcc gcagcgccaa gccggtcacc gagttggtgt gctccggcca
  361 gtgcggcccc gcgcggctgc tgcccaacgc catcgggcgc gtgaagtggt ggcgcccgaa
  421 cggaccggat ttccgctgca tcccggatcg ctaccgcgcg cagcgggtgc agctgctgtg
  481 ccccgggggc gcggcgccgc gctcgcgcaa ggtgcgtctg gtggcctcgt gcaagtgcaa
  541 gcgcctcacc cgcttccaca accagtcgga gctcaaggac ttcgggccgg agaccgcgcg
  601 gccgcagaag ggtcgcaagc cgcggcccgg cgcccgggga gccaaagcca accaggcgga
  661 gctggagaac gcctactaga gcgagcccgc gcctatgcag cccccgcgcg atccgattcg
  721 ttttcagtgt aaagcctgca gcccaggcca ggggtgccaa actttccaga ccgtgtggag
  781 ttcccagccc agtagagacc gcaggtcctt ctgcccgctg cgggggatgg ggagggggtg
  841 gggttcccgc gggccaggag aggaagcttg agtcccagac tctgcctagc cccgggtggg
  901 atgggggtct ttctaccctc gccggaccta tacaggacaa ggcagtgttt ccaccttaaa
  961 gggaagggag tgtggaacga aagacctggg actggttatg gacgtacagt aagatctact
1021 ccttccaccc aaatgtaaag cctgcgtggg ctagataggg tttctgaccc tgacctggcc
1081 actgagtgtg atgttgggct acgtggttct cttttggtac ggtcttcttt gtaaaatagg
1141 gaccggaact ctgctgagat tccaaggatt ggggtacccc gtgtagactg gtgagagaga
1201 ggagaacagg ggaggggtta ggggagagat tgtggtgggc aaccgcctag aagaagctgt
1261 ttgttggctc ccagcctcgc cgcctcagag gtttggcttc ccccactcct tcctctcaaa
1321 tctgccttca aatccatatc tgggataggg aaggccaggg tccgagagat ggtggaaggg
1381 ccagaaatca cactcctggc cccccgaaga gcagtgtccc gcccccaact gccttgtcat
1441 attgtaaagg gattttctac acaacagttt aaggtcgttg gaggaaactg ggcttgccag
1501 tcacctccca tccttgtccc ttgccaggac accacctcct gcctgccacc cacggacaca
1561 tttctgtcta gaaacagagc gtcgtcgtgc tgtcctctga gacagcatat cttacattaa
1621 aaagaataat acgggggggg gggcggaggg cgcaagtgtt atacatatgc tgagaagctg
1681 tcaggcgcca cagcaccacc cacaatcttt ttgtaaatca tttccagaca cctcttactt
1741 tctgtgtaga ttttaattgt taaaagggga ggagagagag cgtttgtaac agaagcacat
1801 ggaggcgccc ccaggggcct tggccctggt gagtttggcg aactttccat gtgagactca
1861 tccacaaaga ctgaaagccg cgtttttttt ttttaagagt tcagtgacat atttattttc
1921 tcatttaagt tatttatgcc aacatttttt tcttgtagag aaaggcagtg ttaatatcgc
1981 tttgtgaagc
Rat SOST protein (NCBI RefSeq ID: NP_085073):
      1 mqlslapcla cllvhaafva vesqgwqafk ndateiipgl reypeppqel ennqtmnrae
    61 nggrpphhpy dtkdvseysc relhytrfvt dgpcrsakpv telvcsgqcg parllpnaig
  121 rvkwwrpngp dfrcipdryr aqrvqllcpg gaaprsrkvr lvasckckrl trfhnqselk
  181 dfgpetarpq kgrkprprar gakanqaele nay
Rat SOST cDNA (NCBI RefSeq ID:NM_030584):
      1 gaggaccgag tgcccttcct ccttctggca ccatgcagct ctcactagcc ccttgccttg
    61 cctgcctgct tgtacatgca gccttcgttg ctgtggagag ccaggggtgg caagccttca
  121 agaatgatgc cacagaaatc atcccgggac tcagagagta cccagagcct cctcaggaac
  181 tagagaacaa ccagaccatg aaccgggccg agaacggagg cagacccccc caccatcctt
  241 atgacaccaa agacgtgtcc gagtacagct gccgcgagct gcactacacc cgcttcgtga
  301 ccgacggccc gtgccgcagt gccaagccgg tcaccgagtt ggtgtgctcg ggccagtgcg
  361 gccccgcgcg gctgctgccc aacgccatcg ggcgcgtgaa gtggtggcgc ccgaacggac
  421 ccgacttccg ctgcatcccg gatcgctacc gcgcgcagcg ggtgcagctg ctgtgccccg
  481 gcggcgcggc gccgcgctcg cgcaaggtgc gtctggtggc ctcgtgcaag tgcaagcgcc
  541 tcacccgctt ccacaaccag tcggagctca aggacttcgg acctgagacc gcgcggccgc
  601 agaagggtcg caagccgcgg ccccgcgccc ggggagccaa agccaaccag gcggagctgg
  661 agaacgccta ctag
```

The identification of CeCan1 (emb|Z74032.1|CEF35B12) as an ortholog of both gremlin and PRDC in the nematode Caenorhabditis elegans (17) and in Ciona intestinalis (Table 2) suggests that the CAN family is of ancient origin. As summarized in Table 2, gremlin and PRDC can be found in all model organisms examined except fly (only the DAN ortholog was found in fly (gi|24648796)). Of interest, analyses of gene structures indicated that all proteins encoded by orthologous gremlin and PRDC genes are derived from a single exon (FIG. 4B). Five amino acids upstream of the cystine-knot domain is a putative proteolytic cleavage site (RKY or RRY) that is highly conserved (Table 3).

Orthologs for cerberus and coco can be found in Xenopus tropicalis and Fugu rubripes, but are missing in invertebrates (Table 2). The mouse dante (17) gene corresponds to a fragment of the mouse coco ortholog (FIG. 4A). In Fugu rubripes, there is only one ortholog for both cerberus and coco. All orthologous genes for cerberus and coco have two exons; the first eight amino acids of the cystine-knot domain are encoded by the 3′ end of the first exon and the remainder of the motif by the second exon (FIG. 4B). In some orthologs, a predicted proteolytic cleavage site can be found upstream of the beginning of the cystine-knot domain (Table 3).

The DAN protein can be found in all model organisms except Caenorhabditis elegans (Table 3). This gene has three exons that encode the cystine-knot domain (except for fly) (FIG. 4B). The human, mouse, and Xenopus orthologs have been cloned (15, 40, 60, 61) and share greater than 90% identity. A carboxyl terminal proline-rich region in DAN could be involved in protein-protein interactions.

The USAG-1 and SOST genes are missing in fly and nematode. A single ortholog for both USAG-1 and SOST was found in Fugu rubripes and Ciona intestinalis (Table 2). All orthologous genes for USAG-1 and SOST have two exons that encode the cystine-knot domain starting at six amino acids downstream of the second exon (FIG. 4B).

Based on their cystine-knot structure (Table 1), overall sequence similarity (FIG. 4A), and conserved exon-intron arrangement (FIG. 4B), the CAN family are divided into four subgroups: 1) gremlin and PRDC; 2) cerberus and coco; 3) DAN; and 4) USAG-1 and SOST. This subdivision is consistent with the phylogenetic tree shown in FIG. 4C. Three-dimensional structures of the eight-membered-ring BMP antagonists.

The region of homology shared by the CAN family genes closely resembles the cystine-knot motif found in members of the TGF-beta superfamily. Crystallographic analysis of the hCG-beta structure suggested that this hormone subunit has six disulfide bonds; three form the cystine-knot structure and the remaining three form intra-subunit bonds as shown (FIG. 5A) (62, 63). Based on the homology between the CAN family of BMP antagonists and hCG-beta (FIG. 5A), assisted by a three-dimensional structure prediction server http://www.sbg.bio.ic.ac.uk/˜3dpssm/ (64), the structure of three key members of the CAN family, gremlin, DAN, and USAG-1, were analyzed (FIG. 5).

Similar to hCG-beta, all CAN family members have an additional cysteine residue in each loop with the potential to form a disulfide bond between loops 1 and 2, leading to a more stable and compact cystine knot (FIGS. 5B, C, and D, dashed line between the two loops). Furthermore, gremlin has an additional cysteine residue near the cystine knot (FIG. 5B, arrow) that could be the basis for homodimerization. Further, DAN has two more cysteine residues that are close enough to form an additional intra-subunit disulfide bond (FIG. 5C, arrows). In contrast, USAG-1 does not have extra cysteine residues and, therefore, is unlikely to form a covalent dimer using disulfide bonds. Sclerostin is the closely related paralog of USAG-1, and the recombinant sclerostin protein was secreted as a monomer (57). Further studies are needed to reveal post-translational modifications of these BMP antagonists.

Based on analyses of the genome sequences from several vertebrates and invertebrates, a comparative genomic view of BMP antagonists emerges. These cystine-knot-containing proteins play important roles during development, organogenesis, and tissue growth and differentiation. The above-described approach allows the identification of three subfamilies of the BMP antagonists and four CAN subgroups. Elucidation of the orthologous and paralogous relationships of genes in this family not only allows a unified nomenclature and classification for these proteins but also provides clues for the future analysis of their functions using model organisms. Regular expression analysis, coupled with comparative genomic approaches, represents a useful paradigm for the investigation of cystine-knot-containing genes in published and emerging genome sequences.

### IV. Generation of Derepressors

TGF derepressors of the instant invention can be generated through site-directed mutagenesis of selected BMP residues that are important for BMP receptor binding, but relatively unimportant for binding to Cystine-knot proteins. As described above, these residues of BMPs can be selected based on comparing the crystal structure of BMP in complex with Cystine-knot protein (e.g. noggin) or BMP receptors.

Examples of three regions that may be altered in BMP-5 and BMP-6 are shown in FIGS. 4 and 5. Other known TGF-beta family members that may be altered are shown in the alignment in FIG. 6 and in the table below. The alignment of FIG. 6 may be used to deduce the appropriate regions for alteration in other family members to generate derepressors. BMP-2 may, for example, be modified at one or more residues selected from the group consisting of D30, W31, A34, H39, F49, P50, D53, S88, L100 and E109. The mutation may be, for example, one or more mutations selected from the group consisting of D30A, W31A, A34D, H39D, F49A, P50A, D53A, S88A, L100A, and E109R.

TABLE 1
Examples of TGF-beta superfamily members known in the art.

| Name | Exemplary References |
|---|---|
| BMP-2 | Wozney et al. (1988) Science 242: (1528-1534 |
| BMP-3 | Wozney et al. (1988) Science 242: (1528-1534 |
| BMP-4 | Wozney et al. (1988) Science 242: (1528-1534 |
| BMP-5 | Celeste et al. (1990) Proc. Natl. Acad. Sci. USA. 87: 9843-9847 |
| BMP-6 | Celeste et al. (1990) Proc. Natl. Acad. Sci. USA. 87: 9843-9847 |
| BMP-7 (OP-1) | Celeste et al. (1990) Proc. Natl. Acad. Sci. USA. 87: 9843-9847 |
| BMP-8 (OP-2) | Ozkaynak et al. (1992) J. Biol. Chem. 267: 25220-25227 |
| BMP-10 | Neuhaus et al., Mech. Dev. 80 (2), 181-184 (1999) |
| BMP-15 (GDF-9B) | Dube et al. Molec. Endocr. 12: 1809-1817, 1998. |
| OP-3 | Ozkaynak et al. PCT/WO94/10203 SEQ ID NO: 1 |
| GDF-1 | Lee (1990) Mol. Endocrinol. 4: 1034-1040 |
| GDF-3 | Caricasole et al., Oncogene 16: 95-103, 1998; |
| | McPherron et al. (1993) J. Biol. Chem. 268: 3444-3449 |
| GDF-5 (CDMP-1) | Hotten et al., Biochem Biophys Res Commun. 1994 Oct |
| | 28; 204(2): 646-52. |
| GDF-6 (BMP-13) | Storm et al., Nature. 1994 Apr 14; 368(6472): 639-43. |
| GDF-7 (BMP-12) | Storm et al., Nature. 1994 Apr 14; 368(6472): 639-43. |
| GDF-8 | McPherron et al., Nature. 1997 May 1; 387(6628): 83-90. |
| GDF-9 | McGrath et al., Molec. Endocr. 9: 131-136, 1995; |
| | McPherron et al. (1993) J. Biol. Chem. 268: 3444-3449 |
| GDF-10 (BMP-3B) | Hino et al. Biochem. Biophys. Res. Commun. 223: 304-310, 1996. |
| GDF-11 (BMP-11) | Nakashima et al., Mech Dev. 1999 February; 80(2): 185-9. |
| GDF-15 (MIC-1) | Bootcov et al., Proc Natl Acad Sci USA. 1997 Oct |
| | 14; 94(21): 11514-9. |
| TGF-β1 | Derynck et al. (1987) Nuci. Acids. Res. 15; 3187 |
| TGF-β2 | Burt et al. (1991) DNA Cell Biol. 10: 723-734 |
| TGF-β3 | ten Dijke et al. (1988) Proc. Natl. Acad. Sci. USA 85: 4715-4719; |
| | Derynck et al. (1988) EMBO J. 7: 3737-3743 |
| TGF-β4 | Burt et al.(1992) Mol. Endcrinol. 6: 989-922 |
| TGF-β5 | Kondaiah et al.(1990) J. Biol. Chem. 265: 1089-1093 |

In another embodiment, random mutagenesis of selected BMP proteins may be employed to identify mutants of diminished BMPR binding, yet substantially unchanged or even increased Cystine-knot binding. The advantage of this approach is that there is no need to predict which residues might be important for either BMPR or Cystine-knot binding beforehand.

Specifically, the ability to employ in vitro mutagenesis or combinatorial modifications of sequences encoding proteins allows for the production of libraries of proteins which can be screened for binding affinity for different ligands. For example, one can randomize a sequence of 1 to 5, 5 to 10, or 10 or more codons, at one or more sites in a DNA sequence encoding a binding protein, make an expression construct and introduce the expression construct into a unicellular microorganism, and develop a library of modified sequences. One can then screen the library for binding affinity of the encoded polypeptides to one or more ligands. The best affinity sequences which are compatible with the cells into which they would be introduced can then be used as the ligand binding domain for a given ligand. The ligand may be evaluated with the desired host cells to determine the level of binding of the ligand to endogenous proteins. A binding profile may be determined for each such ligand which compares ligand binding affinity for the modified ligand binding domain to the affinity for endogenous proteins. Those ligands which have the best binding profile could then be used as the ligand. Phage display techniques, as a non-limiting example, can be used in carrying out the foregoing.

To illustrate, U.S. Pat. No. 6,171,820 describes a rapid and facilitated method of producing from a parental template polynucleotide, a set of mutagenized progeny polynucleotides whereby at each original codon position there is produced at least one substitute codon encoding each of the 20 naturally encoded amino acids. Accordingly, the patent also provides a method of producing from a parental template polypeptide, a set of mutagenized progeny polypeptides wherein each of the 20 naturally encoded amino acids is represented at each original amino acid position. The method provided is termed “sitesaturation mutagenesis,” or simply “saturation mutagenesis,” and can be used in combination with other mutagenization processes described above. This method can be adapted to fine-tune/optimize the final chosen TGF derepressors, so that they exhibit desired biological property including diminished/eliminated BMP receptor binding, with simultaneously increased binding to Cystine-knot proteins.

Alternatively, Hatta et al. (Biopolymers 55(5):399-406, 2000) reported a general scheme for identifying mutant BMP receptors that fail to bind their respective BMP ligands. To identify the amino acid residues on BMP type I receptor responsible for its ligand binding, the structure-activity relationship of the extracellular ligand-binding domain of the BMP type IA receptor (sBMPR-IA) was investigated by alanine-scanning mutagenesis. The mutant receptors, as well as sBMPR-IA, were expressed as fusion proteins with thioredoxin in Escherichia coli, and purified using reverse phase high performance liquid chromatography (RP-HPLC) after digestion with enterokinase. Structural analysis of the parent protein and representative mutants in solution by CD showed no detectable differences in their folding structures. The binding affinity of the mutants to BMP-4 was determined by surface plasmon resonance biosensor. All the mutant receptors examined, with the exception of Y70A, displayed reduced affinities to BMP-4 with the rank order of decreases: I52A (17-fold) approximately F75A (15-fold)>>T64A (4-fold)=T62A (4-fold) approximately E54A (3-fold). The decreases in binding affinity observed for the latter three mutants are mainly due to decreased association rate constants while alterations in rate constants both, for association and dissociation, result in the drastically reduced affinities for the former two mutants. These results indicate that sBMPR-IA recognizes the ligand using the concave face of the molecule. The major ligand-binding site of the BMP type IA receptor consists of Phe75 in loop 2 and Ile52, Glu54, Thr62 and Thr64 on the three-stranded beta-sheet. This study provides a general basis for the ligand/type I receptor recognition in the TGF-beta superfamily. The study also provides a general scheme to identify corresponding BMP ligand (as opposed to BMP receptor) point mutations, which lead to reduced or eliminated BMP receptor binding, yet maintain proper ligand folding structure and thus other ligand characteristics, such as binding to proteins like SOST.

In certain embodiments, the present disclosure contemplates specific mutations of the c TGF-beta superfamily protein sequences so as to create a derepressor type protein. Such mutations may create new glycosylation sites in the polypeptide, as shown in FIGS. 4 and 5. Such mutations may be selected so as to introduce one or more glycosylation sites, such as O-linked or N-linked glycosylation sites. Asparagine-linked glycosylation recognition sites generally comprise a tripeptide sequence, asparagine-X-threonine (where “X” is any amino acid) which are specifically recognized by appropriate cellular glycosylation enzymes. The alteration may also be made by the addition of, or substitution by, one or more serine or threonine residues to the sequence of the wild-type TGF-beta superfamily protein (for O-linked glycosylation sites). The sequence of a derepressor protein may be adjusted, as appropriate, depending on the type of expression system used, as mammalian, yeast, insect and plant cells may all introduce differing glycosylation patterns that can be affected by the amino acid sequence of the peptide.

### V. Preparations of TGF Derepressors

The TGF derepressors of the instant invention can be made as recombinant proteins using standard molecular biology techniques. The term “recombinant protein” refers to a polypeptide of the present invention which is produced by recombinant DNA techniques, wherein generally, DNA encoding a TGF derepressor therapeutic is inserted into a suitable expression vector which is in turn used to transform a host cell to produce the heterologous protein.

Using recombinant techniques well known in the art, the TGF derepressors of the present invention can be produced by standard biological techniques or by chemical synthesis. For example, a host cell transfected with a nucleic acid vector directing expression of a nucleotide sequence encoding the subject polypeptides can be cultured under appropriate conditions to allow expression of the peptide to occur. The recombinant TGF derepressor may be secreted and isolated from a mixture of cells and medium. Alternatively, the protein may be retained cytoplasmically by removing the signal peptide sequence from the recombinant gene and the cells harvested, lysed and the protein isolated. A cell culture includes host cells, media and other byproducts. Suitable media for cell culture are well known in the art. The TGF derepressor can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide.

Appropriate expression constructs for TGF derepressor can be produced by ligating nucleic acid encoding the protein into a vector suitable for expression in either prokaryotic cells, eukaryotic cells, or both. Expression vectors for production of recombinant forms of the subject TGF derepressor include plasmids and other vectors. For instance, suitable vectors for the expression of a TGF derepressor include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due to the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17.

In some instances, it may be desirable to express the recombinant TGF derepressor by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

### VI. Exemplary Nucleic Acid Compositions of the TGF Derepressors

Another aspect of the invention provides expression vectors for expressing the subject TGF derepressor entities in host animals or cultured cells/tissues. For instance, expression vectors are contemplated which include a nucleotide sequence encoding a polypeptide TGF derepressor, which coding sequence is operably linked to at least one transcriptional regulatory sequence. Regulatory sequences for directing expression of the instant polypeptide TGF derepressors are art-recognized and are selected by a number of well understood criteria. Exemplary regulatory sequences are described in Goeddel; Gene Expression Technology Methods in Enzymology, Academic Press, San Diego, Calif. (1990). For instance, any of a wide variety of expression control sequences that control the expression of a DNA sequence when operatively linked to it may be used in these vectors to express DNA sequences encoding the polypeptide TGF derepressors of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast α-mating factors and other sequences known to control the expression of genes of prokaryotic or eukaryotic cells or their viruses, and various combinations thereof. It should be understood that the design of the expression vector may depend on such factors as the choice of the target host cell to be transformed. Moreover, the vector's copy number, the ability to control that copy number and the expression of any other protein encoded by the vector, such as antibiotic markers, should also be considered.

As will be apparent, the subject gene constructs can be used to cause expression of the subject polypeptide TGF derepressors in cells propagated in culture, e.g. to produce proteins or polypeptides, including polypeptide TGF derepressors, for purification.

This invention also pertains to a host cell transfected with a recombinant gene in order to express one of the subject polypeptides. The host cell may be any prokaryotic or eukaryotic cell. For example, a polypeptide of the present invention may be expressed in bacterial cells such as E. coli, insect cells (baculovirus), yeast, or mammalian cells. Other suitable host cells are known to those skilled in the art.

Accordingly, the present invention farther pertains to methods of producing the subject polypeptide TGF derepressors. For example, a host cell transfected with an expression vector encoding a protein of interest can be cultured under appropriate conditions to allow expression of the protein to occur. The protein may be secreted, by inclusion of a secretion signal sequence, and isolated from a mixture of cells and medium containing the protein.

Alternatively, the protein may be retained cytoplasmically and the cells harvested, lysed and the protein isolated. A cell culture includes host cells, media and other byproducts.

Suitable media for cell culture are well known in the art. The proteins can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the protein.

Thus, a coding sequence for a polypeptide of the present invention can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures.

Expression vehicles for production of a recombinant protein include plasmids and other vectors. For instance, suitable vectors for the expression of polypeptide TGF derepressors include plasmids of the types: pBR322-derived plasmids, pEMBL-derived plasmids, pEXderived plasmids, pBTac-derived plasmids and pUC-derived plasmids for expression in prokaryotic cells, such as E. coli. For details, see above.

In yet other embodiments, the subject expression constructs are derived by insertion of the subject gene into viral vectors including recombinant retroviruses, adenovirus, adeno-associated virus, and herpes simplex virus-1, or recombinant bacterial or eukaryotic plasmids. As described in greater detail below, such embodiments of the subject expression constructs are specifically contemplated for use in various in vivo and ex vivo gene therapy protocols.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a polypeptide TGF derepressor of the present invention, rendering the retrovirus replication defective. The replication defective retroviras is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9 9.14 and other standard laboratory manuals. Examples of suitable retroviruses include pLJ, pZIP, pWE and pEM which are well known to those skilled in the art. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eghtis lo et al., (1985) Science 230:1395-1398; Danos and Mulligan, (1988) PNAS USA 85:64606464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345. and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications W093/25234, W094/06920, and W094/11524). For instance, strategies for the modification of the infection spectrum of retroviral vectors include: coupling antibodies specific for cell surface antigens to the viral env protein (Roux et al., (1989) PNAS USA 86: 9079-9083; Julan et al., (1992) J. Gen Virol 73:3251-3255; and Goud et al., (1983) Virology 163: 251-254); or coupling cell surface figands to the viral env proteins (Neda et al., (1991) J. Biol. Chem. 266: 14143-14146).

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6. 616. Rosenfeld et al., (1991) Science 252: 431-434; and Rosenfeld et al., (1992) Cell 68: 155). Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 9 0:2812-2816) and in muscle cells (Quantin et al, (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; HajAhmand and Graham (1986) J. Virol. 57:267). Most replication-defective adenoviral vectors currently in use and therefore favored by the present invention are deleted for all or parts of the viral Et and E3 genes but retain as much as 80% of the adenoviral genetic material (see, e.g., Jones et al., (1979) Cell 16:683; Berkner et al., supra; and Graham et 2o al., in Methods in Molecular Biology, E. J. Murray, Ed. (Humana, Clifton, N.J., 1991) vol. 7. pp. 109-127). Expression of the inserted chimeric gene can be under control of, for example, the E1A promoter, the major late promoter (MLP) and associated leader sequences, the viral E3 promoter, or exogenously added promoter sequences.

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb.

An AAV vector such as that described in Tratschin et al., (1985) Mol. Cell. Biol. 5:32513260 can be used to introduce DNA into cells. A variety of nucleic acids have been introduced into different cell types using AAV vectors (see for example Hernio-nat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

The AAV-based vector has been used successfully in BMP-2 gene therapy (Chen et al., Gene therapy for new bone formation using adeno-associated viral bone morphogenetic protein-2 vectors. Gene Ther. 2003 August; 10(16):1345-53) and BMP-4 gene therapy (Luk et al., Adeno-associated virus-mediated bone morphogenetic protein-4 gene therapy for in vivo bone formation. Biochem Biophys Res Commun. 2003 Aug. 29; 308(3):636-45).

Other viral vector systems that may have application in gene therapy have been derived from herpes virus, vaccinia virus, and several RNA viruses. In particular, herpes virus vectors may provide a unique strategy for persistence of the recombinant gene in cells of the central nervous system and ocular tissue (Pepose et al., (1994) Invest Opthalmol V is Sci 35:2662-2666) In addition to viral transfer methods, such as those illustrated above, non-viral methods can also be employed to cause expression of a protein in the tissue of an animal.

Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly-lysine conjugates, and artificial viral envelopes.

In clinical settings, the gene delivery systems can be introduced into a patient by any of a number of methods, each of which is familiar in the art.

For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al, (1994) PNAS USA 91: 3054-3057).

### VII. Exemplary Pharmaceutical Preparations/Formulations

In certain embodiments, TGF derepressors of the present invention are formulated as therapeutic agents with pharmaceutically acceptable carrier(s). Such therapeutic agents can be administered alone or as a component of a pharmaceutical formulation (composition). The subject compositions may be used alone, or as part of a conjoint therapy with other compounds/pharmaceutical compositions.

Over the years, regulatory health agencies such as FDA of the US government has set increasingly stringent standards for intravenous administration of pharmaceutical compositions to a human, with regard to sterility, non-pyrogenicity and freedom from extraneous particulate matter of the composition. In fact, the standards now set are of such a high level that as a practical matter they could, heretofore, be achieved economically only at the complex, costly, and centralized plant facilities. Thus, in order to administer the therapeutic composition of the instant invention in mammals, including human and other non-human mammals, the therapeutic composition is preferably prepared as pharmaceutical compositions that are substantially free of pyrogenic materials, preferably using ultrapure, pyrogen-free water. As used herein, “ultrapure, pyrogen-free” water is water that has a specific electrical resistance of at least 18 million ohms/cm (18 meg ohms/cm), and contains no pyrogens. Traditionally, water of such purity has been produced by distillation, which removes pyrogens, preceded by mixed-bed deionization. Indeed, the U.S. Food and Drug Administration (“FDA”) requires that water for injection be water purified by either distillation or reverse osmosis. As used herein, “water for injection” is defined as water that satisfies the FDA's purity requirements for water for injection. Distillation, however, is a very energy consuming and costly method for producing water for injection. U.S. Pat. No. 4,548,716 provides a method for producing ultra-pure pyrogen-free water by treatment with pure ozone after filtering, sterilising and deionising. Various other ways of producing pyrogen-free water are described in U.S. Pat. Nos. 4,280,912, 4,069,153, and 4,230,571.

The term “sterile” and “sterilizing” as used herein is not according to the classical definition formulated by the Council on Pharmacy and Chemistry of the American Medical Association, but rather means the absence (or killing) of undesirable microorganisms within the limits prescribed by the United States Pharmacopia XIX (Page 592) for intravenously administrable fluid compositions.

The term “pyrogen-free” as used herein means a material which will provide a negative reaction in the well-known limulus test for the detection of pyrogens and which is meets the requirements of the well-known rabbit test as described in the U.S. Pharmacopia, supra.

U.S. Pat. No. 4,282,863 describes methods of preparing and using intravenous compositions (including proteins, polynucleotides, polysaccharide, etc.) as stable, dry packaged, sterile composition useful for reconstitution to yield pyrogen-free intravenous solutions. The entire content of which is incorporated herein by reference.

The compounds may be formulated for administration in any convenient way for use in human or veterinary medicine (e.g., for the treatment of live stock, such as cow, sheep, goat, pig, and horse, etc., or domestic animals, e.g., cats and dogs.). In certain embodiments, the compound included in the pharmaceutical preparation may itself be active, or may be a prodrug. The term “prodrug” refers to compounds which, under physiological conditions, are converted into therapeutically active agents. For example, the TGF derepressors may be delivered in a form with the intact pro-domain.

Methods of invention may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a therapeutic at a particular target site.

The TGF derepressor therapeutics for use in the subject methods may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the therapeutics disclosed herein, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Co., Easton, Pa., USA 1985). These vehicles include injectable “deposit formulations.”

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the TGF derepressor therapeutics include those suitable for oral/nasal, topical, parenteral and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Methods of preparing these formulations or compositions include combining a TGF derepressor therapeutic agent and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an artemisinin-related compound as an active ingredient. An artemisinin-related compound may also be administered as a bolus, electuary or paste.

In solid dosage forms for oral administration (capsules, tablets, pills, dragees, powders, granules, and the like), one or more TGF derepressor therapeutics of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the invention may be administered topically, either to skin or to mucosal membranes such as those on the cervix and vagina. This offers the greatest opportunity for direct delivery to target tissue (such as broken bone) with the lowest chance of inducing side effects. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to an artemisinin-related compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a soluble ephrin therapeutic, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more TGF derepressor therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more TGF derepressor therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations for intravaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Optionally, such formulations suitable for vaginal administration also include pessaries, tampons, creams, gels, pastes, foams or spray formulations containing such carriers as are known in the art to be appropriate.

The pharmaceutical compositions according to the present invention may be administered as either a single dose or in multiple doses. The pharmaceutical compositions of the present invention may be administered either as individual therapeutic agents or in combination with other therapeutic agents. The treatments of the present invention may be combined with conventional therapies, which may be administered sequentially or simultaneously. The pharmaceutical compositions of the present invention may be administered by any means that enables the TGF derepressor to reach the targeted cells/tissues/organs. In some embodiments, routes of administration include those selected from the group consisting of oral, intravesically, intravenous, intraarterial, intraperitoneal, local administration into the blood supply of the organ in which the targeted cells reside or directly into the cells. Intravenous administration is the preferred mode of administration. It may be accomplished with the aid of an infusion pump.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastermal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, intravesically, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular therapeutic employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other non-human mammals such as equines, cattle, swine and sheep; and poultry and pets in general.

Combined with certain formulations, the subject TGF derepressors can be effective soluble agents. The TGF derepressor can be provided as a fusion peptide along with a second peptide which promotes solubility. To illustrate, the TGF derepressors of the present invention can be provided as part of a fusion polypeptide with all or a fragment of the hinge or Fc portion of the immunoglobulin, which can promote solubility and/or serum stability. The TGF derepressors may also be delivered with a pro-domain sequence of a BMP protein (either covalently linked, such as through a disulfide bond, or non-covalently associated; and either from a heterologous BMP or the same BMP from which the TGF derepressor is derived).

The present invention also contemplates a peptidomimetic sequence of the subject TGF derepressor as described herein.

The TGF derepressors of the instant invention, when used to treat conditions characterized by reduced bone density, may be delivered to bone tissues through the use of a bone-targeting moiety, so as to increase local delivery and/or efficacy of the TGF derepressors to osteoblasts relative to the TGF derepressors alone.

Suitable bone-targeted molecules are those, when used as a component of the subject drug conjugates result in at least a portion of the conjugate, specifically the β-adrenergic agents of the conjugate being delivered to bone. In other words, suitable bone-targeted molecules, when associated with a therapeutic agent, result in exertion of the pharmacological effects of the agent preferentially on bone, in this case, osteoblasts. The targeting molecules suitably include chemical functionalities exhibiting target specificity, e.g., hormones (e.g., biological response modifiers), and antibodies (e.g., monoclonal or polyclonal antibodies), or antibody fragments having the requisite target specificity, e.g., to specific cell-surface antigens.

The therapeutic preparations for use in treating bone disorders may include tetracyclines, calcein, calcitonin, bisphosphonates, chelators, phosphates, polyphosphates, pyrophosphates, phosphonates, diphosphonates, tetraphosphonates, phosphonites, imidodiphosphates, polyaspartic acids, polyglutamic acids, aminophosphosugars, estrogen, peptides known to be associated with mineral phase of bone such as osteonectin, bone sialoprotein and osteopontin, protein with bone mineral binding domains, osteocalcin and osteocalcin peptides, and the like.

The therapeutic preparations for use in treating bone disorders may also include peptides of a repetitive acidic amino acid which may work as a carrier for β-adrenergic agents. Examples of suitable small acidic peptides include, but are not limited to, Asp oligopeptides, Glu oligopeptides, gamma-carboxylated Glu (Gla) oligopeptides, as well as peptides comprising a combination of Asp, Glu and Gla. (Asp)₆ or (Glu)₆ are examples of Asp oligopeptides and Glu oligopeptides.

The bone-targeted molecules may also include molecules which themselves affect bone resorption and bone formation rates, such as bisphosphonates, estrogens and other steroids, such as dehydroepiandrosterone (DHEA). These bone-targeted molecules may have affinity for bone and also possess bone growth therapeutic properties and/or result in a synergistic or additive effect with the β-adrenergic agents on bone resorption or formation. Examples of such molecules are bisphosphonates and fluorides.

The following section gives a more in-depth description of some bone-targeted molecules used to form the conjugated drugs of the present invention.

1. Bisphosphonates

Bisphosphonates are synthetic compounds containing two phosphonate groups bound to a central (geminal) carbon. Two characteristics of bisphosphonates make them desirable bone-targeted molecules. First, bisphosphonates have affinity for bone: they are osteoselectively taken up by bone tissue. Bone scanning agents based on the use of some bisphosphonate compounds have been used in the past to achieve desirable high definition bone scans (see e.g., U.S. Pat. No. 4,810,486 to Kelly et. al). Second, bisphosphonates are useful therapeutic agents for bone diseases. They are capable of inhibiting bone loss, believed to act in a manner which hinders the activity of osteoclasts, so that bone loss is diminished. They are useful in treating bone diseases. including Paget's Disease, osteoporosis, rheumatoid arthritis, and osteoarthritis (see e.g., U.S. Pat. No. 5,428,181 to Sugioka et. al).

Bisphosphonates contain two additional chains (R-1 and R-2, respectively) bound to a central geminal carbon. The availability of two side chains allows numerous substitutions and the development of a variety of analogs with different pharmacological properties. The activity varies greatly from compound to compound, the newest bisphosphonates being 5,000 to 10,000 times more active than etidronate, the first bisphosphonate described. The mechanism of action involves:

a) a direct effect on the osteoclast activity;

b) direct and indirect effects on the osteoclast recruitment, the latter mediated by cells of the osteoblastic lineage and involving the production of an inhibitor of osteoclastic recruitment; and

c) a shortening of osteoclast survival by apoptosis. Large amounts of bisphosphonates can also inhibit mineralization through a physicochemical inhibition of crystal growth. The R-1 structure, together with the P—C—P are primarily responsible for binding to bone mineral and for the physicochemical actions of the bisphosphonates. A hydroxyl group at R-1 provides optimal conditions for these actions. The R-2 is responsible for the antiresorptive action of the bisphosphonates and small modifications or conformational restrictions of this part of the molecule result in marked differences in antiresorptive potency. The presence of a nitrogen function in an alkyl chain or in a ring structure in R-2 greatly enhances the antiresorptive potency and specificity of bisphosphonates for bone resorption and most of the newer potent bisphosphonates contain a nitrogen in their structure.

The terms “bisphosphonate” and “bisphosphonates”, as used herein, are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Non-limiting examples of bisphosphonates useful herein include the following: Alendronic acid, 4-amino-1-hydroxybutylidene-1,1-bisphosphonic acid. Alendronate (also known as alendronate sodium or monosodium trihydrate), 4-amino-1-hydroxybutylidene-1,I-bisphosphonic acid monosodium trihydrate. Alendronic acid and alendronate are described in U.S. Pat. No. 4,922,007, to Kieczykowski et al., issued May 1, 1990, and U.S. Pat. No. 5,019,651, to Kieczykowski, issued May 28, 1991, both of which are incorporated by reference herein in their entirety. Cycloheptylaminomethylene-1,1-bisphosphonic acid, YM 175, Yamanouchi (cimadronate), as described in U.S. Pat. No. 4,970,335, to Isomura et al., issued Nov. 13, 1990, which is incorporated by reference herein in its entirety. 1-dichloromethylene-1,1-diphosphonic acid (clodronic acid), and the disodium salt (clodronate, Procter and Gamble), are described in Belgium Patent 672,205 (1966) and J. Org. Chem. 32, 4111 (1967), both of which are incorporated by reference herein in their entirety. 1-hydroxy(1-pyrrolidinyl)-propylidene-1,1-bisphosphonic acid (EB-1053). 1-hydroxyethane-I,I-diphosphonic acid (etidronic acid). 1-hydroxy(N-methyl-N-pentylamino)propylidene-1,1bisphosphonic acid, also known as BM-210955, Boehringer-Mannheim (ibandronate), is described in U.S. Pat. No. 4,927,814, issued May 22, 1990, which is incorporated by reference herein in its entirety. 6-amino-1-hydroxyhexylidene-1,1-bisphosphonic acid (nen'dronate). 3-(dimethylamino)-1-hydroxypropylidene-1,1-bisphosphonic acid (olpadronate). 3-amino-1-hydroxypropylidene-I,I-bisphosphonic acid (pamidronate). [2-(2-pyridinyl)ethylidene]-I,I-bisphosphonic acid (piridronate) is described in U.S. Pat. No. 4,761,406, which is incorporated by reference in its entirety. 1-hydroxy(3-pyridinyl)-ethylidene-1,1-bisphosphonic acid (risedronate). (4-chlorophenyl)thlomethane-I,I-disphosphonic acid (tiludronate) as described in U.S. Pat. No. 4,876,248, to Breliere et al., Oct. 24, 1989, which is incorporated by reference herein in its entirety. 1-hydroxy(IH-imidazol yl)ethylidene-1,1-bisphosphonic acid (zolendronate). Preferred are bisphosphonates selected from the group consisting of alendronate, cimadronate, clodronate, tiludronate, etidronate, ibandronate, neridronate, risedronate, piridronate, pamidronate, zoledronate, pharmaceutically acceptable salts or esters thereof, and mixtures thereof. More preferred is alendronate, ibandronate, risedronate, pharmaceutically acceptable salts or esters thereof, and mixtures thereof. More preferred is alendronate, pharmaceutically acceptable salts thereof, and mixtures thereof. Most preferred is alendronate monosodium trihydrate. In other embodiments, other preferred salts are the sodium salt of ibandronate, and risedronate monosodium hemi-pentahydrate (i.e. the 2.5 hydrate of the monosodium salt). See WO02/98354, the content of which is incorporated by reference in its entirety herein.

2. Fluorides

Fluoride is another example of a bi-functional bone-targeted molecule. Fluorides can be taken up by bone, and exert a biphasic action at the level of osteoblasts, on bone mineral, on bone structure and function. Fluorides have been used to treat osteoporosis, alone or in combination with anti-resorptive agents. Rubin and Bilezikian, Endocrinol. Metab. Clin. North. Am., 32: 285-307; Pak et al., Trends Endocrinol. Metab. 6: 229-34.

Fluorides used in the present invention may be in the form of sodium fluoride. The term sodium fluoride refers to sodium fluoride in all its forms (e.g., slow release sodium fluoride, sustained release sodium fluoride). Sustained release sodium fluoride is disclosed in U.S. Pat. No. 4,904,478, the disclosure of which is hereby incorporated by reference. The activity of sodium fluoride is readily determined by those skilled in the art according to biological protocols (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296).

3. Small Acidic Peptides

The therapeutic preparations for use in treating bone disorders may also be a small acidic peptide. Hydroxyapatite (HA), a major inorganic component and constituent in the matrix of hard tissues such as bone and teeth, may act as a specific site in targeting bone tissue, to which a small acidic peptide may show affinity.

For example, several bone noncollagenous proteins having repeating sequences of acidic amino acids (Asp or Glu) in their structures have an affinity for and tend to bind to hydroxyapatite (HA). Osteopontin and bone sialoprotein, two major noncollagenous proteins in bone, have an Asp and Glu repeating sequence, respectively. Both osteopontin and bone sialoprotein have a strong affinity for and rapidly bind to HA. Therefore, conjugating β-adrenergic antagonist moieties with peptides associated with these and other noncollagenous proteins may be effective in targeting therapeutic delivery of the β-adrenergic antagonist to the bone because of the associated peptides' affinity to H A. (Asp)₆ conjugation may be a particularly effective delivery means because of the high affinity of (Asp)₆ to hydroxyapatite (HA), however (Glu)₆ may be just as effective.

In contrast to bisphosphonate conjugation, acidic peptides used in peptide conjugation tend to degrade in the resorption process, and may show no pharmacological effect. With bisphosphonate conjugation, the treated tissue tends to exhibit some biphosphonate effect. See US 20030129194, the content of which is incorporated by reference in its entirety.

In another embodiment, TGF derepressor therapeutics of the invention can be administered to animals in animal feed. For example, these compounds can be included in an appropriate feed premix, which is then incorporated into the complete ration in a quantity sufficient to provide a therapeutically effective amount to the animal. Alternatively, an intermediate concentrate or feed supplement containing the TGF derepressor therapeutic agents can be blended into the feed. The way in which such feed premixes and complete rations can be prepared and administered are described in reference books (see, e.g., “Applied Animal Nutrition,” W.H. Freedman and CO., San Francisco, U.S.A., 1969 or “Livestock Feeds and Feeding,” 0 and B books, Corvallis, Ore., U.S.A., 1977).

### VII. Exemplary Disease Conditions Treatable by TGF Derepressors

The subject TGF derepressors can be used to treat any disease conditions where BMP activity is lesser-than-desired. The following are a few exemplary (but not limiting) conditions that could be treated or alleviated by the administration of the subject pharmaceutical compositions containing TGF derepressors. Aside from their use in the treatment of osteoporosis as described above, the TGF repressor compositions may also be used in the following conditions.

Hallahan et al. (Nat. Med. 2003 August; 9(8):1033-8. Epub 2003 Jul. 20) reported that retinoids cause extensive apoptosis of medulloblastoma cells. In a xenograft model, retinoids largely abrogated tumor growth. Using receptor-specific retinoid agonists, they defined a subset of mRNAs that were induced by all active retinoids in retinoid-sensitive cell lines, and identified BMP-2 as a candidate mediator of retinoid activity. They showed that BMP-2 protein induced medulloblastoma cell apoptosis, whereas the BMP-2 antagonist noggin blocked both retinoid and BMP-2-induced apoptosis. BMP-2 also induced p38 mitogen-activated protein kinase (MAPK), which is necessary for BMP-2- and retinoid-induced apoptosis. Retinoid-resistant medulloblastoma cells underwent apoptosis when treated with BMP-2 or when cultured with retinoid-sensitive medulloblastoma cells. Retinoid-induced expression of BMP-2 is thus necessary and sufficient for apoptosis of retinoid-responsive cells, and expression of BMP-2 by retinoid-sensitive cells is sufficient to induce apoptosis in surrounding retinoid-resistant cells. Thus the subject TGF derepressors can be used to increase BMP-2 activity to achieve the same result.

Waite and Eng (Hum Mol. Genet. 2003 Mar. 15; 12(6):679-84) recently reported that exposure to BMP2 increased the tumor supressor PTEN protein levels in a time- and dose-dependent manner. The increase in PTEN protein was rapid and was not due to an increase in new protein synthesis, as cycloheximide treatment did not inhibit BMP2-induced PTEN accumulation, suggesting that BMP2 stimulation inhibited PTEN protein degradation. In addition, BMP2 treatment of MCF-7 cells decreased the association of PTEN with two proteins in the degradative pathway, UbCH7 and UbC9. These data indicate that BMP2 exposure can regulate PTEN protein levels by decreasing PTEN's association with the degradative pathway. This opens up a new mode of treating diseases resulting from reduced PTEN activity such as Cowden syndrome, Bannayan-Riley-Ruvalcaba syndrome and Proteus syndrome.

Glaucoma is a blinding disease usually associated with high intraocular pressure (IOP). In some families, abnormal anterior segment development contributes to glaucoma. The genes causing anterior segment dysgenesis and glaucoma in most of these families are not identified and the affected developmental processes are poorly understood. Chang et al. (BMC Genet. 2001; 2(1):18. Epub 2001 Nov. 6.) studied the importance of Bmp4 gene dosage for ocular development and developmental glaucoma, and found that Bmp4+/− (haploinsufficient) mice have anterior segment abnormalities including malformed, absent or blocked trabecular meshwork and Schlemm's canal drainage structures. Mice with severe drainage structure abnormalities, over 80% or more of their angle's extent, have elevated IOP. On the C57BL/6J background there is also persistence of the hyaloid vasculature, diminished numbers of inner retinal cells, and absence of the optic nerve. Thus heterozygous deficiency of BMP4 results in anterior segment dysgenesis and elevated IOP. The abnormalities are similar to those in human patients with developmental glaucoma. Thus, insufficient BMP4 is a strong candidate to contribute to Axenfeld-Rieger anomaly and other developmental conditions associated with human glaucoma. BMP4 also participates in posterior segment development and wild-type levels are usually critical for optic nerve development on the C57BL/6J background. The subject TGF derepressors may be used to increase BMP4 activity to treat those conditions.

Mathura et al. (Invest Opthalmol V is Sci. 2000 February; 41(2):592-600) showed that, in mice with inherited photoreceptor degeneration, there was a dramatic decrease in BMP4 mRNA in retina and RPE (retinal pigmented epithelium) during and after the degeneration. Thymidine incorporation in early-passage RPE cells showed a 14-fold stimulation above control with 5% serum that was decreased to 322%, 393%, and 313% in the presence of BMP-2 (10 ng/ml), BMP4 (10 ng/ml), and transforming growth factor (TGF)-beta1 (2 ng/ml), respectively. Thus BMP-2 and BMP-4 may serve as negative growth regulators in the retina and RPE that are downregulated by injury, to allow tissue repair. Modulation of expression of the BMPs may provide a means to control the exaggerated wound repair that occurs in proliferative retinopathies.

Marker et al. (Genomics. 1995 Aug. 10; 28(3):576-80) places BMP-7 within a chromosome region thought to contain one or more unidentified imprinted genes. A direct test suggests that BMP7 is not imprinted. An examination of embryonic RNA expression patterns shows that Bmp7 is expressed in a variety of skeletal and nonskeletal tissues. Both embryonic expression patterns and the human chromosomal sublocalization inferred from its mouse location make BMP7 a likely candidate for the gene affected in some patients with Holt-Oram syndrome.

Congenital obstructive nephropathy is a common disease affecting fetuses and young children. The kidney shows profound morphologic and functional changes. The physiologic developmental kidney program is disturbed in the most advanced cases, arguing for altered temporal/spatial expression of genes which control normal nephrogenesis. Excessive apoptosis is an undisputed mechanism in these processes, mediated by decreased expression of apoptosis inhibiting genes (Bcl-2, HGF, IGF, BMP7), and overexpression of pro-apoptotic genes like Bax and TGF-beta. Thus TGF derepressors of the invention can be administered to boost BMP-7 levels.

Bone morphogenetic protein-7 (BMP7) is highly expressed in renal tubules and generally promotes maintenance of epithelial phenotype. Wang et al. (J Am Soc Nephrol. 2001 November; 12(11):2392-9) reported that at 15 wk of streptozotocin-induced diabetes, renal expression of BMP7 is declined by about half, and it decreased further by 30 wk to <10% of timed controls. During the evolution of diabetic nephropathy, the secreted BMP antagonist gremlin increased substantially. Thus, in experimental diabetic nephropathy, renal tubular BMP7 and some of its receptors decreased and gremlin, a secreted BMP antagonist, increased. The loss of BMP7 activity per se is profibrogenic in tubular cells. Thus the TGF derepressors of the invention can be administered to reverse or alleviate this condition.

Giannobile et al. (J Periodontol. 1998 February; 69(2):129-37) also reported that recombinant human osteogenic protein-1 (OP-1) stimulates periodontal wound healing in class III furcation defects.

Perides et al. (Neurosci Lett. 1995 Feb. 24; 187(1):21-4) studied neuroprotective actions of osteogenic protein-1 (OP-1) in a rat model of cerebral hypoxia/ischemia. Intraperitoneal injection of 50 micrograms of OP-1 prior to bilateral carotid ligation and transient hypoxia in 12-day-old rats reduced cerebral infarct area from 44.8+/−3.3% in vehicle-injected controls to 29+/−4.9%. Treatment of 14-day-old rats with 20 micrograms of OP-1 1 h after hypoxia reduced mortality from 45% to 13%. Thus OP-1 may represent a novel class of neuroprotective agents.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available Immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### REFERENCES CITED

1. Kobayashi Y, Takashima H, Tamaoki H, Kyogoku Y, Lambert P, Kuroda H, Chino N, Watanabe T X, Kimura T, Sakakibara S, et al. 1991 The cystine-stabilized alpha-helix: a common structural motif of ion-channel blocking neurotoxic peptides. Biopolymers 31:1213-20.
2. Tamaoki H, Kobayashi Y, Nishimura S, Ohkubo T, Kyogoku Y, Nakajima K, Kumagaye S, Kimura T, Sakakibara S 1991 Solution conformation of endothelin determined by means of 1H-NMR spectroscopy and distance geometry calculations. Protein Eng 4:509-18.
3. Tamaoki H, Miura R, Kusunoki M, Kyogoka Y, Kobayashi Y, Moroder L 1998 Folding motifs induced and stabilized by distinct cystine frameworks. Protein Eng 11:649-59.
4. Hearn M T, Gomme P T 2000 Molecular architecture and biorecognition processes of the cystine knot protein superfamily: part I. The glycoprotein hormones. J Mol Recognit 13:223-78.
5. Carugo O, Lu S, Luo J, Gu X, Liang S, Strobl S, Pongor S 2001 Structural analysis of free and enzyme-bound amaranth {alpha}-amylase inhibitor: classification within the knottin fold superfamily and analysis of its functional flexibility. Protein Eng. 14:639-646.
6. Craik D J, Daly N L, Waine C 2001 The cystine knot motif in toxins and implications for drug design. Toxicon 39:43-60 17.
7. Vitt U A, Hsu S Y, Hsueh A J W 2001 Evolution and Classification of Cystine Knot-Containing Hormones and Related Extracellular Signaling Molecules. Mol Endocrinol 15:681-694.
8. Chang H, Brown C W, Matzuk M M 2002 Genetic analysis of the mammalian transforming growth factor-beta superfamily. Endocr Rev 23:787-823.
9. Matzuk M M, DeMayo F J, Hadsell L A, Kumar T R 2003 Overexpression of human chorionic gonadotropin causes multiple reproductive defects in transgenic mice. Biol Reprod 69:338-46.
10. Risma K A, Clay C M, Nett T M, Wagner T, Yun J, Nilson J H 1995 Targeted overexpression of luteinizing hormone in transgenic mice leads to infertility, polycystic ovaries, and ovarian tumors. Proc Natl Acad Sci USA 92:1322-6.
11. Wozney J M 2002 Overview of bone morphogenetic proteins. Spine 27:S2-8.
12. Bottner M, Laaff M, Schechinger B, Rappold G, Unsicker K, Suter-Crazzolara C 1999 Characterization of the rat, mouse, and human genes of growth/differentiation factor-15/macrophage inhibiting cytokine-1 (GDF-15/MIC-1). Gene 237:105-11.
13. McPherron A C, Lee S J 1993 GDF-3 and GDF-9: two new members of the transforming growth factor-beta superfamily containing a novel pattern of cysteines. J Biol Chem 268:3444-9.
14. Griffith D L, Keck P C, Sampath T K, Rueger D C, Carlson W D 1996 Three-dimensional structure of recombinant human osteogenic protein 1: structural paradigm for the transforming growth factor beta superfamily. Proc Natl Acad Sci USA 93:878-83.
15. Hsu D R, Economides A N, Wang X, Eimon P M, Harland R M 1998 The Xenopus dorsalizing factor Gremlin identifies a novel family of secreted proteins that antagonize BMP activities. Mol Cell 1:673-83.
16. Piccolo S, Sasai Y, Lu B, De Robertis E M 1996 Dorsoventral patterning in Xenopus: inhibition of ventral signals by direct binding of chordin to BMP-4. Cell 86:589-98.
17. Pearce J J, Penny G, Rossant J 1999 A mouse cerberus/Dan-related gene family. Dev Biol 209:98-110.
18. Topol L Z, Marx M, Laugier D, Bogdanova N N, Boubnov N V, Clausen P A, Calothy G, Blair D G 1997 Identification of drm, a novel gene whose expression is suppressed in transformed cells and which can inhibit growth of normal but not transformed cells in culture. Mol Cell Biol 17:4801-10.
19. Oelgeschlager M, Larrain J, Geissert D, De Robertis E M 2000 The evolutionarily conserved BMP-binding protein Twisted gastrulation promotes BMP signalling. Nature 405:757-763
20. Ross J J, Shimmi O, Vilmos P, Petryk A, Kim H, Gaudenz K, Hermanson S, Ekker S C, O'Connor M B, Marsh J L 2001 Twisted gastrulation is a conserved extracellular BMP antagonist. Nature 410:479-83.
21. Bachiller D, Klingensmith J, Kemp C, Belo J A, Anderson R M, May S R, McMahon J A, McMahon A P, Harland R M, Rossant J, De Robertis E M 2000 The organizer factors Chordin and Noggin are required for mouse forebrain development. Nature 403:658-61.
22. Gratsch T E, O'Shea K S 2002 Noggin and chordin have distinct activities in promoting lineage commitment of mouse embryonic stem (ES) cells. Dev Biol 245:83-94.
23. Dionne M S, Brunet L J, Eimon P M, Harland R M 2002 Noggin is required for correct guidance of dorsal root ganglion axons. Dev Biol 251:283-93.
24. Groppe J, Greenwald J, Wiater E, Rodriguez-Leon J, Economides A N, Kwiatkowski W, Affolter M, Vale W W, Belmonte J C, Choe S 2002 Structural basis of BMP signalling inhibition by the cystine knot protein Noggin. Nature 420:636-42.
25. Warren S M, Brunet L J, Harland R M, Economides A N, Longaker M T 2003 The BMP antagonist noggin regulates cranial suture fusion. Nature 422:625-9.
26. Waterston R H et al., 2002, Initial sequencing and comparative analysis of the mouse genome. Nature 420:520-62.
27. Aparicio S et al., 2002 Whole-genome shotgun assembly and analysis of the genome of Fugu rubripes. Science 297:1301-10.
28. Dehal P et al., 2002 The draft genome of Ciona intestinalis: insights into chordate and vertebrate origins. Science 298:2157-67.
29. Friedl J E F 2002 Mastering regular expressions, 2nd ed. O'Reilly, Sebastopol, C A
30. Nielsen H, Engelbrecht J, Brunak S, von Heijne G 1997 Identification of prokaryotic and eukaryotic signal peptides and prediction of their cleavage sites. Protein Eng 10:1-6.
31. Schultz J, Milpetz F, Bork P, Ponting C P 1998 SMART, a simple modular architecture research tool: identification of signaling domains. Proc Natl Acad Sci USA 95:5857-64.
32. Simmons D G, Kennedy T G 2002 Uterine Sensitization-Associated Gene-1: A Novel Gene Induced Within the Rat Endometrium at the Time of Uterine Receptivity/Sensitization for the Decidual Cell Reaction. Biol Reprod 67:1638-1645.
33. Bell E, Munoz-Sanjuan I, Altmann C R, Vonica A, Brivanlou A H 2003 Cell fate specification and competence by Coco, a maternal BMP, TGFbeta and Wnt inhibitor. Development 130:1381-9.
34. Itasaki N, Jones C M, Mercurio S, Rowe A, Domingos P M, Smith J C, Krumlauf R
2003 Wise, a context-dependent activator and inhibitor of Wnt signalling. Development 130:4295-305.
35. Retief J D 2000 Phylogenetic analysis using PHYLIP. Methods Mol Biol 132:243-58.
36. Belo J A, Bouwmeester T, Leyns L, Kertesz N, Gallo M, Follettie M, De Robertis E M 1997 Cerberus-like is a secreted factor with neutralizing activity expressed in the anterior primitive endoderm of the mouse gastrula. Mech Dev 68:45-57.
37. Biben C, Stanley E, Fabri L, Kotecha S, Rhinn M, Drinkwater C, Lah M, Wang C C, Nash A, Hilton D, Ang S L, Mohun T, Harvey R P 1998 Murine cerberus homologue mCer-1: a candidate anterior patterning molecule. Dev Biol 194:135-51.
38. Bouwmeester T, Kim S, Sasai Y, Lu B, De Robertis E M 1996 Cerberus is a headinducing secreted factor expressed in the anterior endodenn of Spemann's organizer. Nature 382:595-601.
39. Piccolo S, Agius E, Leyns L, Bhattacharyya S, Grunz H, Bouwmeester T, De Robertis E M 1999 The head inducer Cerberus is a multifunctional antagonist of Nodal, BMP and Wnt signals. Nature 397:707-10.
40. Stanley et al., 1998 Natriuretic effect of an adenosine-1 receptor antagonist in cirrhotic patients with ascites. Gastroenterology 115:406-11.
41. Dionne M S, Skarnes W C, Harland R M 2001 Mutation and Analysis of Dan, the Founding Member of the Dan Family of Transforming Growth Factor {beta} Antagonists. Mol. Cell. Biol. 21:636-643.
42. Topol L Z, Modi W S, Koochekpour S, Blair D G 2000 DRM/GREMLIN(CKTSF1B1) maps to human chromosome 15 and is highly expressed in adult and fetal brain. Cytogenet Cell Genet. 89:79-84.
43. Khokha M K, Hsu D, Brunet L J, Dionne M S, Harland R M 2003 Gremlin is the BMP antagonist required for maintenance of Shh and Fgf signals during limb patterning. Nat Genet. 34:303-7.
44. Shi W, Zhao J, Anderson K D, Warburton D 2001 Gremlin negatively modulates BMP-4 induction of embryonic mouse lung branching morphogenesis. Am J Physiol Lung Cell Mol Physiol 280:L1030-1039.
45. McMahon R, Murphy M, Clarkson M, Taal M, Mackenzie H S, Godson C, Martin F, Brady H R 2000 IHG-2, a mesangial cell gene induced by high glucose, is human gremlin. Regulation by extracellular glucose concentration, cyclic mechanical strain, and transforming growth factor-beta1. J Biol Chem 275:9901-4.
46. Lappin D W, McMahon R, Murphy M, Brady H R 2002 Gremlin: an example of the reemergence of developmental programmes in diabetic nephropathy. Nephrol Dial Transplant 17 Suppl 9:65-7.
47. Murphy M, McMahon R, Lappin D W, Brady H R 2002 Gremlins: is this what renal fibrogenesis has come to? Exp Nephrol 10:241-4.
48. Minabe-Saegusa C, Saegusa H, Tsukahara M, Noguchi S 1998 Sequence and expression of a novel mouse gene PRDC (protein related to DAN and cerberus) identified by a gene trap approach. Dev Growth Differ 40:343-53.
49. Rodriguez Esteban C, Capdevila J, Economides A N, Pascual J, Ortiz A, Izpisua Belmonte J C 1999 The novel Cer-like protein Caronte mediates the establishment of embryonic left-right asymmetry. Nature 401:243-51.
50. Belo J A, Bachiller D, Agius E, Kemp C, Borges A C, Marques S, Piccolo S, De Robertis E M 2000 Cerberus-like is a secreted BMP and nodal antagonist not essential for mouse development. Genesis 26:265-70.
51. Yamamoto S, Hikasa H, Ono H, Taira M 2003 Molecular link in the sequential induction of the Spemann organizer: direct activation of the cerberus gene by Xlim-1, Xotx2, Mix. 1, and Siamois, immediately downstream from Nodal and Wnt signaling. Dev Biol 257:190-204.
52. Ozaki T, Sakiyama S 1993 Molecular cloning of rat calpactin I heavy-chain cDNA whose expression is induced in v-src-transformed rat culture cell lines. Oncogene 8:1707-10.
53. Ozaki T, Sakiyama S1994 Tumor-suppressive activity of N03 gene product in v-srctransformed rat 3Y1 fibroblasts. Cancer Res 54:646-8.
54. Ozaki T, Nakamura Y, Enomoto H, Hirose M, Sakiyama S 1995 Overexpression of DAN gene product in normal rat fibroblasts causes a retardation of the entry into the S phase. Cancer Res 55:895-900.
55. Enomoto H, Ozaki T, Takahashi E, Nomura N, Tabata S, Takahashi H, Ohnuma N, Tanabe M, Iwai J, Yoshida H, et al. 1994 Identification of human DAN gene, mapping to the putative neuroblastoma tumor suppressor locus. Oncogene 9:2785-91.
56. White P S, Maris J M, Beltinger C, Sulman E, Marshall H N, Fujimori M, Kaufman B A, Biegel J A, Allen C, Hilliard C, et al. 1995 A region of consistent deletion in neuroblastoma maps within human chromosome 1p36.2-36.3. Proc Natl Acad Sci USA 92:5520-4.
57. Kusu N, Laurikkala J, Imanishi M, Usui H, Konishi M, Miyake A, Thesleff I, Itoh N 2003 Sclerostin is a novel secreted osteoclast-derived bone morphogenetic protein (BMP) antagonist with unique ligand specificity. J. Biol. Chem.:M301716200.
58. Balemans W, Ebeling M, Patel N, Van Hul E, Olson P, Dioszegi M, Lacza C, Wuyts W, Van Den Ende J, Willems P, Paes-Alves A F, Hill S, Bueno M, Ramos F J, Tacconi P, Dikkers F G, Stratakis C, Lindpaintner K, Vickery B, Foernzler D, Van Hul W 2001 Increased bone density in sclerosteosis is due to the deficiency of a novel secreted protein (SOST). Hum. Mol. Genet. 10:537-543.
59. Brunkow M E, Gardner J C, Van Ness J, Paeper B W, Kovacevich B R, Proll S, Skonier J E, Zhao L, Sabo P J, Fu Y, Alisch R S, Gillett L, Colbert T, Tacconi P, Galas D, Hamersma H, Beighton P, Mulligan J 2001 Bone dysplasia sclerosteosis results from loss of the SOST gene product, a novel cystine knot-containing protein. Am J Hum Genet. 68:577-89.
60. Ozaki T, Kondo K, Nakamura Y, Ichimiya S, Nakagawara A, Sakiyama S1997 Interaction of DA41, a DAN-binding protein, with the epidermal growth factor-like protein, S(1-5). Biochem Biophys Res Commun 237:245-50.
61. Ozaki T, Ma J, Takenaga K, Sakiyama S 1996 Cloning of mouse DAN cDNA and its down-regulation in transformed cells. Jpn J Cancer Res 87:58-61.
62. Lapthorn A J, Harris D C, Littlejohn A, Lustbader J W, Canfield R E, Machin K J, Morgan F J, Isaacs N W 1994 Crystal structure of human chorionic gonadotropin. Nature 369:455-61.
63. Wu H, Lustbader J W, Liu Y, Canfield R E, Hendrickson W A 1994 Structure of human chorionic gonadotropin at 2.6 A resolution from MAD analysis of the selenomethionyl protein. Structure 2:545-58.
64. Kelley L A, MacCallum R M, Sternberg M J 2000 Enhanced genome annotation using structural profiles in the program 3D-PSSM. J Mol Biol 299:499-520.
65. Letunic I, Goodstadt L, Dickens N J, Doerks T, Schultz J, Mott R, Ciccarelli F, Copley R R, Ponting C P, Bork P 2002 Recent improvements to the SMART domain-based sequence annotation resource. Nucleic Acids Res 30:242-4.
66. Corpet F 1988 Multiple sequence alignment with hierarchical clustering. Nucleic Acids Res 16:10881-90.
67. Rogers S, Wells R, Rechsteiner M 1986 Amino acid sequences common to rapidly degraded proteins: the PEST hypothesis. Science 234:364-8.
68. Rechsteiner M, Rogers S W 1996 PEST sequences and regulation by proteolysis. Trends Biochem Sci 21:267-71.
69. Chen B, Athanasiou M, Gu Q, Blair D G 2002 Drm/Gremlin transcriptionally activates p21(Cip1) via a novel mechanism and inhibits neoplastic transformation. Biochem Biophys Res Commun 295:1135-41.
70. Lappin D W, Hensey C, McMahon R, Godson C, Brady H R 2000 Gremlins, glomeruli and diabetic nephropathy. Curr Opin Nephrol Hypertens 9:469-72.
71. Wang S N, Lapage J, Hirschberg R 2001 Loss of tubular bone morphogenetic protein-7 in diabetic nephropathy. J Am Soc Nephrol 12:2392-9.

### EQUIVALENTS

A skilled artisan will recognize, or be able to ascertain using no more than routine experimentation, many equivalents to the specific embodiments of the invention described herein. Such equivalents are intended to be encompassed by the following claims.

1. A BMP variant polypeptide that differs by one or more amino acid residues from a wild-type BMP protein, which BMP variant polypeptide (i) binds to and neutralizes one or more cystine knot-containing BMP antagonists, and (ii) has diminished potency, relative to the wild-type BMP protein, for inducing receptor-mediated signal transduction in cells otherwise responsive to the wild-type BMP protein.

2. The BMP variant polypeptide of claim 1, that from the wild-type BMP protein by point mutation, deletion, truncation, insertion and/or chemical modification.

3. The BMP variant polypeptide of claim 1, having an EC₅₀ for inducing said receptor-mediated signal transduction that is at least 5 times greater than the wild-type BMP protein.

4. The BMP variant polypeptide of claim 3, having an EC₅₀ for inducing said receptor-mediated signal transduction that is at least 100 times greater than the wild-type BMP protein.

5. The BMP variant polypeptide of claim 1, having a K_{d} for binding a BMP receptor that is at least 5 fold greater than the wild-type BMP protein.

6. The BMP variant polypeptide of claim 5, having a K_{d} for binding a BMP receptor that is at least 100 fold greater than the wild-type BMP protein.

7. The BMP variant polypeptide of claim 5, having a K_{d} for binding a type I receptor that is at least 100 fold greater than the wild-type BMP protein.

8. The BMP variant polypeptide of claim 1, which binds to a cystine knot-containing BMP antagonists selected from the group consisting of CAN (eight-membered ring), twisted gastrulation (nine-membered ring), chordin and noggin (10-membered ring).

9. The BMP variant polypeptide of claim 1, which binds sclerostin and promotes bone growth and mineralization in vivo.

10. The BMP variant polypeptide of claim 9, which is a sequence variant of BMP-5.

11. The BMP variant polypeptide of claim 10, which varies from wild-type BMP-5 at one or more residues in the regions DLGWQDWIIAPEGYA, FPLNAHMNATNHAIVQTLVHL, or ISVLYFDDSSNVILKKYR.

12. The BMP variant polypeptide of claim 9, which is a sequence variant of BMP-6.

13. The BMP variant polypeptide of claim 12, which varies from wild-type BMP-6 at one or more residues in the regions DLGWQDWIIAPKGYA, FPLNAHMNATNHAIVQTLVHL or ISVLYFDDNSNVILKKYR.

14. The BMP variant polypeptide of claim 9, which is a sequence variant of BMP-2.

15. The BMP variant polypeptide of claim 14, which varies from wild-type BMP-2 at one or more residues selected from the group consisting of D30, W31, A34, H39, F49, P50, D53, S88, L100 and E109.

16. The BMP variant polypeptide of claim 15, having one or more mutations selected from the group consisting of D30A, W31A, A34D, H39D, F49A, P50A, D53A, S88A, L100A, and E109R.

17. The BMP variant polypeptide of claim 1, obtained by random mutagenesis.

18. The BMP variant polypeptide of claim 1, which binds to said cystine knot-containing BMP antagonists with a K_{d} of 1 μM or less.

19. The BMP variant polypeptide of claim 18, which binds to said cystine knot-containing BMP antagonists with a K_{d} of 10 nM or less.

20. The BMP variant polypeptide of claim 1, which is a fusion protein additionally including one more polypeptide portions that enhance one or more of: in vivo stability, in vivo half life, uptake/administration, tissue localization or distribution, formation of protein complexes, and/or purification.

21. The BMP variant polypeptide of claim 20, wherein said fusion protein includes an immunoglobulin Fc domain, preferably, the Fc domain is fused to the N-terminus of said derepressor.

22. The BMP variant polypeptide of claim 20, wherein said fusion protein includes a purification subsequence.

23. The BMP variant polypeptide of claim 20, wherein said purification subsequence is selected from the group consisting of an epitope tag, a FLAG tag, a polyhistidine sequence, and a GST fusion.

24. The BMP variant polypeptide of claim 1, which includes one or more modified amino acid residues selected from: a glycosylated amino acid, a PEGylated amino acid, a prenylated amino acid, an acetylated amino acid, a biotinylated amino acid, an amino acid conjugated to a lipid moiety, and an amino acid conjugated to an organic derivatizing agent.

25. The BMP variant polypeptide of claim 24, wherein said modified amino acid residues enhance one or more of: in vivo stability, in vivo half life, uptake/administration, tissue localization or distribution, formation of protein complexes, and/or purification.

26. The BMP variant polypeptide of claim 1, which is a variant of a BMP family proteins selected from BMP-2 (-2A), BMP-3, BMP-3B (GDF-10), BMP-4 (-2B), BMP-5, BMP-6, BMP-7 (OP-1), BMP-8 (OP-2), BMP-9 (GDF-2), BMP-10, BMP-11 (GDF-11), BMP-12 (GDF-7), BMP-13 (GDF-6), PC8 (OP-3), DPP, 60A, Vg1, Vgr-1, Univin, GDF-5, GDF-3, and GDF-1.

27. A pharmaceutical preparation of a BMP variant polypeptide from any of the above claims, wherein said pharmaceutical preparation is substantially free of pyrogenic materials so as to be suitable for administration as a human or veterinarian therapeutic.

28. A pharmaceutical preparation suitable for use in a mammal, comprising: a vector including: (a) a coding sequence for a BMP variant polypeptide of any of the above claims; (b) transcriptional control sequences for regulating expression of the BMP variant polypeptide in vivo; and optionally (c) a pharmaceutically acceptable carrier and/or transfection agent.

29. A packaged pharmaceutical comprising: (a) a pharmaceutically acceptable preparation of a BMP variant polypeptide of claim 1; and (b) a label or package insert describing use of the preparation to treat a human or veterinary patient.

30. A packaged pharmaceutical comprising: (a) a pharmaceutically acceptable preparation of a BMP variant polypeptide of claim 9, which binds sclerostin and promotes bone growth and mineralization in vivo; and (b) a label or package insert describing use of the preparation to promote increase of bone density and/or reduce the rate of loss of bone density in a human or veterinary patient.

31. A method for promoting BMP signal transduction comprising administering the BMP variant polypeptide of claim 1.

32. A method for promoting bone density and/or reducing the rate of loss of bone density in a human or veterinary patient comprising administering the BMP variant polypeptide of claim 9.

33. The method of claim 32, wherein the BMP variant polypeptide is administered in an amount effective to reduce the severity of a pathological condition which is characterized, at least in part, by an reduced bone density or increased rate of loss of bone density.

34. The method of claim 32, which is part of a treatment or prevention of a bone disease selected from: osteoporosis, osteopenia, Paget's disease, osteomalacia, renal osteodystrophy, periodontal disease, and localized bone loss associated with periprosthetic osteolysis.

35. The method of claim 34, wherein the osteoporosis is post-menopausal osteoporosis, steroid-induced osteoporosis, male osteoporosis, disease-induced osteoporosis, or idiopathic osteoporosis.

36. The method of claim 32, wherein the BMP variant polypeptide is co-administered with one or more other agents that inhibits bone resorption.

37. The method of claim 32, wherein the BMP variant polypeptide is associated with a bone-targeting moiety.

38. The method of claim 32, wherein the bone-targeting moiety is a bisphosphonate, a fluoride, a small acidic peptide, an antibody against specific bone proteins, a metal ion selected from Sr²⁺, Zn²⁺, Mg²⁺, Fe²⁺, Cu²⁺, Mn²⁺, Ca²⁺, Cu²⁺, Co²⁺, Cr²⁺ or Mo²⁺, a tracer, or a heterocyclic molecule with high bone affinity.

39. The BMP variant polypeptide of claim 9, wherein the BMP variant polypeptide is associated with a bone-targeting moiety.

40. The BMP variant polypeptide of claim 39, wherein the bone-targeting moiety is a bisphosphonate, a fluoride, a small acidic peptide, an antibody against specific bone proteins, a metal ion selected from Sr²⁺, Zn²⁺, Mg²⁺, Fe²⁺, Cu²⁺, Mn²⁺, Ca²⁺, Cu²⁺, Co²⁺, Cr²⁺ or Mo²⁺, a tracer, or a heterocyclic molecule with high bone affinity.

41. The use of the BMP variant polypeptide of claim 9 for the preparation of a medicament for promoting bone density and/or reducing the rate of loss of bone density in a human or veterinary patient.


SEQUENCE LISTING (112 sequences)

SEQ ID NO: 1 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 2 - DNA, 4046 bp, Homo sapiens
gcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttagcaagagatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcactgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcttcaagcctgaggttttatatacaaactccctgaatactctttttgccttgtatcttctcagcctcctagccaagtcctatgtaatatggaaaacaaacactgcagacttgagattcagttgccgatcaaggctctggcattcagagaacccttgcaactcgagaagctgtttttatttcgtttttgttttgatccagtgctctcccatctaacaactaaacaggagccatttcaaggcgggagatattttaaacacccaaaatgttgggtctgattttcaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccagcaaacactgaatttctcttgttgttttaactctgccacaagaatgcaatttcgttaacggagatgacttaagttggcagcagtaatcttcttttaggagcttgtaccacagtcttgcacataagtgcagatttggctcaagtaaagagaatttcctcaacactaacttcactgggataatcagcagcgtaactaccctaaaagcatatcactagccaaagagggaaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagatatttgtggtcttgatcatacctattaaaataatgccaaacaccaaatatgaattttatgatgtacactttgtgcttggcattaaaagaaaaaaacacacatcctggaagtctgtaagttgttttttgttactgtaggtcttcaaagttaagagtgtaagtgaaaaatctggaggagaggataatttccactgtgtggaatgtgaatagttaaatgaaaagttatggttatttaatgtaattattacttcaaatcctttggtcactgtgatttcaagcatgttttctttttctcctttatatgactttctctgagttgggcaaagaagaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaaagagcaactaataaattaaacctattctttcaaaaaaaaa

SEQ ID NO: 3 - protein, 168 aa, Homo sapiens
MFWKLSLSLFMVAVLVKVAEARKNRPAGAIHSPYKDGSSNNSERWQHQIKEVLASSQEALVVTERKYLKSDWCKTQPLRQTVSEEGCRSRTILNRFCYGQCNSFYIPRHVKKEEESFQSCAFCKPQRVTSVLVELECPGLDPPFRLKKIQKVKQCRCMSVNLSDSDKQ

SEQ ID NO: 4 - DNA, 3208 bp, Homo sapiens
agcgggctctcgcctctcctgcaccctcagccggcgcgcttctcttatgggcgtctgctgcagtctggctgcggtcgaactgaaagcggcggcgggagaccaaacttagaccccgctgtggactagagaactcagagaaggcagagggagagggagagagagagagagaagggacccgaggaggaggcttccatcacgtcattgcaggatgttctggaagctttccctgtccttgttcatggtggcggtgctggtgaaggtggcggaagcccggaagaaccggccggcgggcgccatccactcgccttacaaggacggcagcagcaacaactcggagagatggcagcaccagatcaaggaggtactggcctccagccaggaggccctggtggtcaccgagcgcaagtacctcaagagtgactggtgcaagacgcagccgctgcggcagacggtgagcgaggagggctgccggagccgcaccatcctcaaccgcttctgctacggccagtgcaactccttctacatcccgcggcacgtgaagaaggaggaggagtccttccagtcctgcgccttctgcaagccccagcgcgtcacctccgtcctcgtggagctcgagtgccccggcctggacccacccttccgactcaagaaaatccagaaggtgaagcagtgccggtgcatgtccgtgaacctgagcgactcggacaagcagtgagcgccgggcaggacgcagctcagccccgcgcgcgcccggcagctgggtggcgccgccgccgcctctgtccctgccctccgagcccactgtcacgctgcctggtgttccccatcagcagcaagcacttctcttagggctgacggtgtccttgtcacagacgtggatcgcaagtggagcttttgctgatgtgttcctgaccgaccccgggtcccacctgtgcccctaaaccaggcggtggatcaggcccgaaggaaaatgctggaaaaccaccaccgccaccgagcagtcagagaggacctttcgaggtgatgcactgtaaatccggcattgatgtttccgccatgtggaaaagcaaggatttccctggccccgccctgcactccagctcgcccctttcccgcgcgaagtgaaggatgcgtcacttttctgagcggcccctcagacctctcctgctggtccctggacctcttgctgggagcgctcattcttgtgactggcaggctgccctcggtcgctgctgtgatgaggatatgtgcaacctccataaatatgtcggtggacggggcgtctcctaataaacctgataccaagaagacaacttgagccacttggatcccgaagtgggcttcccgggaaacctcgagcagagcaggctacacaatcccccatcctcaagtccccaccgcctcttcttcctcactgtccccgccccctatttgtggactaaagatgaactctggtgtgcatgctattattgctgcaattagaatattatcacacacaaaggtctctatattaacgctggttttataagtgaccgtatattgtaaagagctgttaaaaagtattatagacctattcatgtattatttcacatgttttgactctggcttgcagcaccattcggagtaaggatgacagcaggcccagaaggtgttttactcagaaaaaaaaatgcctggtcagcaattcactgtcatgcacctatttttagattgggcagggagatgggaggagtcgtgatttttactttgaatactatttcactgacgttactaagtattgcagcacaatgtagaaattggcttgggatggataggtatagggaagggcattcgttgggaaattgatgaccggaaactagaaagcccctataatgtggaatatgcatatgtggggtgaaaccgtatactactttcactgccatgacactaggcaaaaaatatttcctgttaaaagaaagagaaaaagaaagggatattgaaaaatataaggagttgagtcgctttgtggtatagaagggagtaaggaataaaagatatagaatagtaatctagttgaactgttctgcacaaaacatgcttccttttcaaagaatagacttcaaaagggacgaacaaacattaggtcaccttttgtgtttataatgaattgttgacgtttccatgctgtggtctcttgatatacggaagtagacgtaacttattttccagcgaaattatatacttcttctgggctcctttgctcagttgccccaggaaggcttccctgtgagtaatgcttatggaatctgacaaatgtgaatgagctgccattggtgggaatcagtaaactttgaaaacctttcactgatgattccctggtaagtgctcctgtgaggggccacttccctagtatgtgagagagaccctctgcaagccgctgtggggacttagcttcctgatactaggtagtgatggtggatctagagacattcacttgaagagcaaggattggggtggggtgggttggtgaatctatctcaggccactgcactgcaaggtgcatttctgtcatttctgcaacaccatgagacctgagcaagaatgtgctgtggcagagggggtcttggtttcataaatgggtatatacagcctgaactcaggaggcagaaatcaaaagaaatccttgagggctggaacctgaggatagagcgaaaatggagagaagggtgttcaaggtcaggagtgatttttgcatttggtggaaaacctcaagtcagactcagtggcagaacatgcagcagaaaagctgtgaaggaaggaaatttacgtgattgtaatggaggctaggggtggattgggaggtgagtggaggagagtctctgaccagctgcaaagccaacccctatcattttattaacatggaacccacacagataggggagggattaagctaagaacttacagaatgcaaacacgagcacactctcttcgaacccaattgtgggtgtagcaatgaaagcaatatgatatgctgcagtgtgaagctccttctggggttatcgtatgtacaaagtttaccttataatggctcaaattgtatttaatttttttgttttgtttataaatgaagaaaagctataagtataatgtaattattttataggtatactattaaattatagaacaaataaagataatctaggattatatgcgaaaaaaaaaaaaaaaa

SEQ ID NO: 5 - protein, 267 aa, Homo sapiens
MHLLLFQLLVLLPLGKTTRHQDGRQNQSSLSPVLLPRNQRELPTGNHEEAEEKPDLFVAVPHLVATSPAGEGQRQREKMLSRFGRFWKKPEREMHPSRDSDSEPFPPGTQSLIQPIDGMKMEKSPLREEAKKFWHHFMFRKTPASQGVILPIKSHEVHWETCRTVPFSQTITHEGCEKVVVQNNLCFGKCGSVHFPGAAQHSHTSCSHCLPAKFTTMHLPLNCTELSSVIKVVMLVEECQCKVKTEHEDGHILHAGSQDSFIPGVSA

SEQ ID NO: 6 - DNA, 804 bp, Homo sapiens
atgcatctcctcttatttcagctgctggtactcctgcctctaggaaagaccacacggcaccaggatggccgccagaatcagagttctctttcccccgtactcctgccaaggaatcaaagagagcttcccacaggcaaccatgaggaagctgaggagaagccagatctgtttgtcgcagtgccacaccttgtagccaccagccctgcaggggaaggccagaggcagagagagaagatgctgtccagatttggcaggttctggaagaagcctgagagagaaatgcatccatccagggactcagatagtgagcccttcccacctgggacccagtccctcatccagccgatagatggaatgaaaatggagaaatctcctcttcgggaagaagccaagaaattctggcaccacttcatgttcagaaaaactccggcttctcagggggtcatcttgcccatcaaaagccatgaagtacattgggagacctgcaggacagtgcccttcagccagactataacccacgaaggctgtgaaaaagtagttgttcagaacaacctttgctttgggaaatgcgggtctgttcattttcctggagccgcgcagcactcccatacctcctgctctcactgtttgcctgccaagttcaccacgatgcacttgccactgaactgcactgaactttcctccgtgatcaaggtggtgatgctggtggaggagtgccagtgcaaggtgaagacggagcatgaagatggacacatcctacatgctggctcccaggattcctttatcccaggagtttcagcttga

SEQ ID NO: 7 - protein, 189 aa, Homo sapiens
MLLGQLSTLLCLLSGALPTGSGRPEPQSPRPQSWAAANQTWALGPGALPPLVPASALGSWKAFLGLQKARQLGMGRLQRGQDEVAAVTLPLNPQEVIQGMCKAVPFVQVFSRPGCSAIRLRNHLCFGHCSSLYIPGSDPTPLVLCNSCMPARKRWAPVVLWCLTGSSASRRRVKISTMLIEGCHCSPKA

SEQ ID NO: 8 - DNA, 1731 bp, Homo sapiens
agtccggacagacagacaggcagacagacgcacggacaagcagatgctccttggccagctatccactcttctgtgcctgcttagcggggccctgcctacaggctcagggaggcctgaaccccagtctcctcgacctcagtcctgggctgcagccaatcagacctgggctctgggcccaggggccctgcccccactggtgccagcttctgcccttgggagctggaaggccttcttgggcctgcagaaagccaggcagctggggatgggcaggctgcagcgtgggcaagacgaggtggctgctgtgactctgccgctgaaccctcaggaagtgatccaggggatgtgtaaggctgtgcccttcgttcaggtgttctcccggcccggctgctcagccatacgcctccgaaatcatctgtgctttggtcattgctcctctctctacatccctggctcggaccccaccccactagtcctgtgcaacagctgtatgcctgctcgcaagcgttgggcacccgtggtcctgtggtgtctcactggcagctcagcctcccgtcgacgggtgaagatatccaccatgctgatcgaggggtgtcactgcagcccaaaagcatgaactgagcatcgtggatgggtgcacggagacacgcaccttggagaaatgaggggagatggaccaagaaagacgtggacctggatgatgtactctgggtcaagagaccagggatgcagggttaggcagacaggtccccagagtcctcaccctgctccccagacagtagacacagtgcccgtcctggagttgcaccactgatagtcacagcacacaatgattgacaactcacttttttttttttttttgagatggagtctcgctctgtcgcccaggctggagtgcagtggcgcaatctcagctcactgcaagctccacctcccgggtttatgccattctcctgtctcagcctcccgagtagctgggactacaggcacccgccaacacgcccggctaatttttcgtatttttagtaaagacagggtttcaccgtgttagccaggatggtctctatctcctgacctcgtgatctgcctgccttggccttattattttttttttttaaggacagagtctctctctgtcacccaggctggagtgcaatggcgcgatcttggctcactgtaacttccacttgccaggctcaagcagttctcctgcctcagcctcctgagtagctgggactacaggcacccgccaccatgcccagctaatttttgtatttttagtagagacagagtttcaccatattagcctggctggtctcaaactcctggcctcaggtgatctgcccacctcggcctcccaaagtgctgggatcaaatccactgttaatcattaggctgaactgtctcttatagaatgaggtcaaagacactcccagttgcagggagggtagatggccccacccagaccgagagacacagtgatgacctcagcctagggacaccaaaaaaaaaaaaaaaaaaaacccaaaccaaaaacgcaaaccaaagcaggcaggcagacagctgctgggggaaatcctggggtccttgagacagaggcaggaccctcgtgttcccagctgcctcttgccttgatagtggtgctgtgtccctctcagaccccccacctgagtctccacagagccccacgcctggcatggcattccacagaaaccataaaggttggctgagtcc

SEQ ID NO: 9 - protein, 180 aa, Homo sapiens
MLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 10 - DNA, 2056 bp, Homo sapiens
cgcagcgcagcccagccgagcgtcgcggggccgccccccgccctgccggccgcctcgccgagcctcctggggcgcccgggcccgcgacccccgcacccagctccgcaggaccggcgggcgcgcgcgggctctggaggccacgggcatgatgcttcgggtcctggtgggggctgtcctccctgccatgctactggctgccccaccacccatcaacaagctggcactgttcccagataagagtgcctggtgcgaagccaagaacatcacccagatcgtgggccacagcggctgtgaggccaagtccatccagaacagggcgtgcctaggacagtgcttcagctacagcgtccccaacaccttcccacagtccacagagtccctggttcactgtgactcctgcatgccagcccagtccatgtgggagattgtgacgctggagtgcccgggccacgaggaggtgcccagggtggacaagctggtggagaagatcctgcactgtagctgccaggcctgcggcaaggagcctagtcacgaggggctgagcgtctatgtgcagggcgaggacgggccgggatcccagcccggcacccaccctcacccccatccccacccccatcctggcgggcagacccctgagcccgaggacccccctggggccccccacacagaggaagagggggctgaggactgaggcccccccaactcttcctcccctctcatccccctgtggaatgttgggtctcactctctggggaagtcaggggagaagctgaagcccccctttggcactggatggacttggcttcagactcggacttgaatgctgcccggttgccatggagatctgaaggggcggggttagagccaagctgcacaatttaatatattcaagagtggggggaggaagcagaggtcttcagggctctttttttggggggggtggtctcttcctgtctggcttctagagatgtgcctgtgggagggggaggaagttggctgagccattgagtgctgggggaggccatccaagatggcatgaatcgggctaaggtccctgggggtgcagatggtactgctgaggtcccgggcttagtgtgagcatcttgccagcctcaggcttgagggagggctgggctagaaagaccactggcagaaacaggaggctccggcccacaggtttccccaaggcctctcaccccacttcccatctccagggaagcgtcgccccagtggcactgaagtggccctccctcagcggaggggtttgggagtcaggcctgggcaggaccctgctgactcgtggcgcgggagctgggagccaggctctccgggcctttctctggcttccttggcttgcctggtgggggaaggggaggaggggaagaaggaaagggaagagtcttccaaggccagaaggagggggacaaccccccaagaccatccctgaagacgagcatccccctcctctccctgttagaaatgttagtgccccgcactgtgccccaagttctaggccccccagaaagctgccagagccggccgccttctcccctctcccagggatgctctttgtaaatatcggatgggtgtgggagtgaggggttacctccctcgccccaaggttccagaggccctaggcgggatgggctcgctgaacctcgaggaactccaggacgaggaggacatgggacttgcgtggacagtcagggttcacttgggctctctctagctccccaattctgcctgcctcctccctcccagctgcactttaaccctagaaggtggggacctggggggagggacagggcaggcgggcccatgaagaaagcccctcgttgcccagcactgtctgcgtctgctcttctgtgcccagggtggctgccagcccactgcctcctgcctggggtggcctggccctcctggctgttgcgacgcgggcttctggagcttgtcaccattggacagtctccctgatggaccctcagtcttctcatgaataaattccttcaacgccaaaaaaaaaaaaaaaaaaaaaaaaaaaaaaaaaaaaaaaaaa

SEQ ID NO: 11 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 12 - DNA, 2323 bp, Homo sapiens
agagcctgtgctactggaaggtggcgtgccctcctctggctggtaccatgcagctcccactggccctgtgtctcgtctgcctgctggtacacacagccttccgtgtagtggagggccaggggtggcaggcgttcaagaatgatgccacggaaatcatccccgagctcggagagtaccccgagcctccaccggagctggagaacaacaagaccatgaaccgggcggagaacggagggcggcctccccaccacccctttgagaccaaagacgtgtccgagtacagctgccgcgagctgcacttcacccgctacgtgaccgatgggccgtgccgcagcgccaagccggtcaccgagctggtgtgctccggccagtgcggcccggcgcgcctgctgcccaacgccatcggccgcggcaagtggtggcgacctagtgggcccgacttccgctgcatccccgaccgctaccgcgcgcagcgcgtgcagctgctgtgtcccggtggtgaggcgccgcgcgcgcgcaaggtgcgcctggtggcctcgtgcaagtgcaagcgcctcacccgcttccacaaccagtcggagctcaaggacttcgggaccgaggccgctcggccgcagaagggccggaagccgcggccccgcgcccggagcgccaaagccaaccaggccgagctggagaacgcctactagagcccgcccgcgcccctccccaccggcgggcgccccggccctgaacccgcgccccacatttctgtcctctgcgcgtggtttgattgtttatatttcattgtaaatgcctgcaacccagggcagggggctgagaccttccaggccctgaggaatcccgggcgccggcaaggcccccctcagcccgccagctgaggggtcccacggggcaggggagggaattgagagtcacagacactgagccacgcagccccgcctctggggccgcctacctttgctggtcccacttcagaggaggcagaaatggaagcattttcaccgccctggggttttaagggagcggtgtgggagtgggaaagtccagggactggttaagaaagttggataagattcccccttgcacctcgctgcccatcagaaagcctgaggcgtgcccagagcacaagactgggggcaactgtagatgtggtttctagtcctggctctgccactaacttgctgtgtaaccttgaactacacaattctccttcgggacctcaatttccactttgtaaaatgagggtggaggtgggaataggatctcgaggagactattggcatatgattccaaggactccagtgccttttgaatgggcagaggtgagagagagagagagaaagagagagaatgaatgcagttgcattgattcagtgccaaggtcacttccagaattcagagttgtgatgctctcttctgacagccaaagatgaaaaacaaacagaaaaaaaaaagtaaagagtctatttatggctgacatatttacggctgacaaactcctggaagaagctatgctgcttcccagcctggcttccccggatgtttggctacctccacccctccatctcaaagaaataacatcatccattggggtagaaaaggagagggtccgagggtggtgggagggatagaaatcacatccgccccaacttcccaaagagcagcatccctcccccgacccatagccatgttttaaagtcaccttccgaagagaagtgaaaggttcaaggacactggccttgcaggcccgagggagcagccatcacaaactcacagaccagcacatcccttttgagacaccgccttctgcccaccactcacggacacatttctgcctagaaaacagcttcttactgctcttacatgtgatggcatatcttacactaaaagaatattattgggggaaaaactacaagtgctgtacatatgctgagaaactgcagagcataatagctgccacccaaaaatctttttgaaaatcatttccagacaacctcttactttctgtgtagtttttaattgttaaaaaaaaaaagttttaaacagaagcacatgacatatgaaagcctgcaggactggtcgtttttttggcaattcttccacgtgggacttgtccacaagaatgaaagtagtggtttttaaagagttaagttacatatttattttctcacttaagttatttatgcaaaagtttttcttgtagagaatgacaatgttaatattgctttatgaattaacagtctgttcttccagagtccagagacattgttaataaagacaatgaatcatgaccgaaagaaaaaaaaaaaaaaaaaaaaaa

SEQ ID NO: 13 - protein, 211 aa, Mus musculus
MQPSLAPCLICLLVHAAFCAVEGQGWQAFRNDATEVIPGLGEYPEPPPENNQTMNRAENGGRPPHHPYDAKDVSEYSCRELHYTRFLTDGPCRSAKPVTELVCSGQCGPARLLPNAIGRVKWWRPNGPDFRCIPDRYRAQRVQLLCPGGAAPRSRKVRLVASCKCKRLTRFHNQSELKDFGPETARPQKGRKPRPGARGAKANQAELENAY

SEQ ID NO: 14 - DNA, 1990 bp, Mus musculus
gactggagcctgtgctaccgagtgccctcctccacctggcagcatgcagccctcactagccccgtgcctcatctgcctacttgtgcacgctgccttctgtgctgtggagggccaggggtggcaagccttcaggaatgatgccacagaggtcatcccagggcttggagagtaccccgagcctcctcctgagaacaaccagaccatgaaccgggcggagaatggaggcagacctccccaccatccctatgacgccaaagatgtgtccgagtacagctgccgcgagctgcactacacccgcttcctgacagacggcccatgccgcagcgccaagccggtcaccgagttggtgtgctccggccagtgcggccccgcgcggctgctgcccaacgccatcgggcgcgtgaagtggtggcgcccgaacggaccggatttccgctgcatcccggatcgctaccgcgcgcagcgggtgcagctgctgtgccccgggggcgcggcgccgcgctcgcgcaaggtgcgtctggtggcctcgtgcaagtgcaagcgcctcacccgcttccacaaccagtcggagctcaaggacttcgggccggagaccgcgcggccgcagaagggtcgcaagccgcggcccggcgcccggggagccaaagccaaccaggcggagctggagaacgcctactagagcgagcccgcgcctatgcagcccccgcgcgatccgattcgttttcagtgtaaagcctgcagcccaggccaggggtgccaaactttccagaccgtgtggagttcccagcccagtagagaccgcaggtccttctgcccgctgcgggggatggggagggggtggggttcccgcgggccaggagaggaagcttgagtcccagactctgcctagccccgggtgggatgggggtctttctaccctcgccggacctatacaggacaaggcagtgtttccaccttaaagggaagggagtgtggaacgaaagacctgggactggttatggacgtacagtaagatctactccttccacccaaatgtaaagcctgcgtgggctagatagggtttctgaccctgacctggccactgagtgtgatgttgggctacgtggttctcttttggtacggtcttctttgtaaaatagggaccggaactctgctgagattccaaggattggggtaccccgtgtagactggtgagagagaggagaacaggggaggggttaggggagagattgtggtgggcaaccgcctagaagaagctgtttgttggctcccagcctcgccgcctcagaggtttggcttcccccactccttcctctcaaatctgccttcaaatccatatctgggatagggaaggccagggtccgagagatggtggaagggccagaaatcacactcctggccccccgaagagcagtgtcccgcccccaactgccttgtcatattgtaaagggattttctacacaacagtttaaggtcgttggaggaaactgggcttgccagtcacctcccatccttgtcccttgccaggacaccacctcctgcctgccacccacggacacatttctgtctagaaacagagcgtcgtcgtgctgtcctctgagacagcatatcttacattaaaaagaataatacgggggggggggcggagggcgcaagtgttatacatatgctgagaagctgtcaggcgccacagcaccacccacaatctttttgtaaatcatttccagacacctcttactttctgtgtagattttaattgttaaaaggggaggagagagagcgtttgtaacagaagcacatggaggcgcccccaggggccttggccctggtgagtttggcgaactttccatgtgagactcatccacaaagactgaaagccgcgttttttttttttaagagttcagtgacatatttattttctcatttaagttatttatgccaacatttttttcttgtagagaaaggcagtgttaatatcgctttgtgaagc

SEQ ID NO: 15 - protein, 213 aa, Rat
MQLSLAPCLACLLVHAAFVAVESQGWQAFKNDATEIIPGLREYPEPPQELENNQTMNRAENGGRPPHHPYDTKDVSEYSCRELHYTRFVTDGPCRSAKPVTELVCSGQCGPARLLPNAIGRVKWWRPNGPDFRCIPDRYRAQRVQLLCPGGAAPRSRKVRLVASCKCKRLTRFHNQSELKDFGPETARPQKGRKPRPRARGAKANQAELENAY

SEQ ID NO: 16 - DNA, 674 bp, Rat
gaggaccgagtgcccttcctccttctggcaccatgcagctctcactagccccttgccttgcctgcctgcttgtacatgcagccttcgttgctgtggagagccaggggtggcaagccttcaagaatgatgccacagaaatcatcccgggactcagagagtacccagagcctcctcaggaactagagaacaaccagaccatgaaccgggccgagaacggaggcagacccccccaccatccttatgacaccaaagacgtgtccgagtacagctgccgcgagctgcactacacccgcttcgtgaccgacggcccgtgccgcagtgccaagccggtcaccgagttggtgtgctcgggccagtgcggccccgcgcggctgctgcccaacgccatcgggcgcgtgaagtggtggcgcccgaacggacccgacttccgctgcatcccggatcgctaccgcgcgcagcgggtgcagctgctgtgccccggcggcgcggcgccgcgctcgcgcaaggtgcgtctggtggcctcgtgcaagtgcaagcgcctcacccgcttccacaaccagtcggagctcaaggacttcggacctgagaccgcgcggccgcagaagggtcgcaagccgcggccccgcgcccggggagccaaagccaaccaggcggagctggagaacgcctactag

SEQ ID NO: 17 - protein, 15 aa, Homo sapiens
DLGWQDWIIAPEGYA

SEQ ID NO: 18 - protein, 21 aa, Homo sapiens
FPLNAHMNATNHAIVQTLVHL

SEQ ID NO: 19 - protein, 18 aa, Homo sapiens
ISVLYFDDSSNVILKKYR

SEQ ID NO: 20 - protein, 15 aa, Homo sapiens
DLGWQDWIIAPKGYA

SEQ ID NO: 21 - protein, 21 aa, Homo sapiens
FPLNAHMNATNHAIVQTLVHL

SEQ ID NO: 22 - protein, 18 aa, Homo sapiens
ISVLYFDDNSNVILKKYR

SEQ ID NO: 23 - protein, 91 aa, Homo sapiens
CKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 24 - protein, 91 aa, Homo sapiens; identical to SEQ ID NO: 23

SEQ ID NO: 25 - protein, 91 aa, Homo sapiens; identical to SEQ ID NO: 23

SEQ ID NO: 26 - protein, 91 aa, Homo sapiens
CKTQPLKQTIHEDGCNSRTIINRFCYGQCNSFYIPRHIRREEGSFQSCSFCKPKKFTTMVVTLNCPELQPPTKKKRITRVKQCRCISIDLD

SEQ ID NO: 27 - protein, 91 aa, Homo sapiens
CKTQPLKQTIHEEGCVSRTIINRFCYGQCNSFYIPRHIRREEGAFQSCSFCKPKRFTTMTFTLNCPDQQPPTKKKRIQRVKQCRCISIDLD

SEQ ID NO: 28 - protein, 95 aa, Homo sapiens
CKSQPVRQYIRTADGCKGELINQFCYGQCNSFYIPKDIEIDETDYFRFCAFCKPKTEEWISVRLRCRRKGRKKRGRYVIKRVKRVKGCTCISVPE

SEQ ID NO: 29 - protein, 94 aa, Homo sapiens
CKTQPLRQTVSEEGCRSRTILNRFCYGQCNSFYIPRHVKKEEESFQSCAFCKPQRVTSVLVELECPGLDPPFRLKKIQKVKQCRCMSVNLSDSD

SEQ ID NO: 30 - protein, 94 aa, Homo sapiens
CKTQPLRQTVSEEGCRSRTILNRFCYGQCNSFYIPRHVKKEEDSFQSCAFCKPQRVTSVIVELECPGLDPPFRIKKIQKVKECRCMSVNLSDSD

SEQ ID NO: 31 - protein, 94 aa, Homo sapiens
CKTQPLRQTVSEEGCRSRTILNRFCYGQCNSFYIPRHVKKEEDSFQSCAFCKPQRVTSVIVELECPGLDPPFRIKKIQKVKHCRCMSVNLSDSD

SEQ ID NO: 32 - protein, 111 aa, Homo sapiens
CKTQPLRQTISEEGCRSRTVVNRFCYGQCNSFYIPRHMGPSSAQAQSRGQALGSRRKSHNKAQEPFQSCSFCRPHRITQLTVQLDCPDLQPPFKHRKVQRVKQCRCMSVDV

SEQ ID NO: 33 - protein, 89 aa, Homo sapiens
CRTVPFSQTITHEGCEKVVVQNNLCFGKCGSVHFPGAAQHSHTSCSHCLPAKFTTMHLPLNCTELSSVIKVVMLVEECQCKVKTEHEDG

SEQ ID NO: 34 - protein, 89 aa, Homo sapiens
CRTVPFNQTIAHEDCQKVVVQNNLCFGKCSSIRFPGEGADAHSFCSHCSPTKFTTVHLMLNCTSPTPVVKMVMQVEECQCMVKTERGEE

SEQ ID NO: 35 - protein, 89 aa, Homo sapiens
CRTVPFNQTIAHEDCQKVTVQNNLCFGKCGSIHFPGEGAHPHNFCSHCSPTKFTTTHLRLNCTSPTPVVKMVIQVEECQCMVKTEHGEE

SEQ ID NO: 36 - protein, 88 aa, Homo sapiens
CKTLPFTQNIVHENCDRMVIQNNLCFGKCISLHVPNQQDRRNTCSHCLPSKFTLNHLTLNCTGSKNVVKVVMMVEECTCEAHKSNFHQ

SEQ ID NO: 37 - protein, 89 aa, Homo sapiens
CKAVPFVQVFSRPGCSAIRLRNHLCFGHCSSLYIPGSDPTPLVLCNSCMPARKRWAPVVLWCLTGSSASRRRVKISTMLIEGCHCSPKA

SEQ ID NO: 38 - protein, 89 aa, Homo sapiens
CKALSFVQVISRPGCTSARVLNHLCFGRCSSFYIPSSDPTPVVFCNSCVPARKRWTSVTLWCGAGQLASPRRVRISTVLVQKCQCRPKL

SEQ ID NO: 39 - protein, 89 aa, Homo sapiens
CKALPFVQVISRPGCTSARVLNHLCFGHCSSFYIPSSDPSPVVLCNSCVPSRRRWTSVALWCGAGQSASPRRVRISTVLVQKCQCHPRL

SEQ ID NO: 40 - protein, 89 aa, Homo sapiens
CHALPFIQNVFRKNCFPVRLPNKFCFGQCNSFYVPGWPAGLSQPCTSCAPSRSRRISLPLRCRSGHLAWQEVELVEECECETRYDRNTV

SEQ ID NO: 41 - protein, 96 aa, Homo sapiens
CSGVPFTQRVTAAGCSAVTVHNKLCFGQCSSLFVPSEAPLGTGMGLLHHRGPCSRCAPSKAHAVVLPLLCGARVQEKRVLVVEECKCETGREEKAA

SEQ ID NO: 42 - protein, 95 aa, Homo sapiens
CEAKNITQIVGHSGCEAKSIQNRACLGQCFSYSVPNTFPQSTESLVHCDSCMPAQSMWEIVTLECPGHEEVPRVDKLVEKILHCSCQACGKEPSH

SEQ ID NO: 43 - protein, 95 aa, Homo sapiens
CEAKNITQIVGHSGCEAKSIQNRACLGQCFSYSVPNTFPQSTESLVHCDSCMPAQSMWEIVTLECPDHEEVPRVDKLVEKIVHCSCQACGKEPSH

SEQ ID NO: 44 - protein, 95 aa, Homo sapiens
CEAKNITQIVGHSGCEAKSIQNRACLGQCFSYSVPNTFPQSTESLVHCDSCMPAQSMWEIVTLECPGHEEVPRVDKLVEKIVHCSCQACGKEPSH

SEQ ID NO: 45 - protein, 89 aa, Homo sapiens
CEAKNITQIVGHTGCLPQSIQNRACLGQCFSYSVPNTFPQSTESLVHCDSCMPAQTQWEVVTLDCPDSPNVDKLVERIFHCSCQSCSKE

SEQ ID NO: 46 - protein, 95 aa, Homo sapiens
CEAKNITQIVGHSGCESKSIQNRACLGQCFSYSVPNTFPQSTESLVHCDSCMPIDSVWDVVTLECPGNEEFPRVDKLVEKILQCSCQACGKELSQ

SEQ ID NO: 47 - protein, 96 aa, Homo sapiens
CQVTPVIHVLQYPGCVPKPIPSFACVGRCASYIQVSGSKIWQMERSCMCCQESGEREAAVSLFCPKVKPGERKFKKVLTKAPLECMCRPCTSIEES

SEQ ID NO: 48 - protein, 97 aa, Homo sapiens
CRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQ

SEQ ID NO: 49 - protein, 97 aa, Homo sapiens
CRELHYTRFLTDGPCRSAKPVTELVCSGQCGPARLLPNAIGRVKWWRPNGPDFRCIPDRYRAQRVQLLCPGGAAPRSRKVRLVASCKCKRLTRFHNQ

SEQ ID NO: 50 - protein, 97 aa, Homo sapiens
CRELHYTRFVTDGPCRSAKPVTELVCSGQCGPARLLPNAIGRVKWWRPNGPDFRCIPDRYRAQRVQLLCPGGAAPRSRKVRLVASCKCKRLTRFHNQ

SEQ ID NO: 51 - protein, 96 aa, Homo sapiens
CRELRSTRYVTDGSCRSAKPIKELVCSGQCLPAHLMPNTIGRGKWWRSNTSEYRCIPAHSRTRRIQLQCPNGNTRTYKIRIVTSCKCKRFRAHHNQ

SEQ ID NO: 52 - protein, 100 aa, Homo sapiens
CRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNE

SEQ ID NO: 53 - protein, 100 aa, Homo sapiens; identical to SEQ ID NO: 52

SEQ ID NO: 54 - protein, 100 aa, Homo sapiens; identical to SEQ ID NO: 52

SEQ ID NO: 55 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 56 - protein, 20 aa, Homo sapiens
SFRDLGWQDWIIAPKGYAAF

SEQ ID NO: 57 - protein, 21 aa, Homo sapiens
  VARIANT 6: Xaa = Ser or Thr
SFRNLXTWQDWIIAPKGYAAF

SEQ ID NO: 58 - protein, 20 aa, Homo sapiens
  VARIANT 11: Xaa = Ser or Thr
SFRDLGWQNWXIAPKGYAAF

SEQ ID NO: 59 - protein, 30 aa, Homo sapiens
YCDGECSFPLNAHMNATNHAIVQTLVHLMN

SEQ ID NO: 60 - protein, 31 aa, Homo sapiens
  VARIANT 13: Xaa = Ser or Thr
YCDGECSFPLNAXTMNATNHAIVQTLVHLMN

SEQ ID NO: 61 - protein, 30 aa, Homo sapiens
  VARIANT 20: Xaa = Ser or Thr
YCDGECSFPLNAHMNATNHXIVQTLVHLMN

SEQ ID NO: 62 - protein, 30 aa, Homo sapiens
YCDGECSFPLNAHMNATNHAINQTLVHLMN

SEQ ID NO: 63 - protein, 29 aa, Homo sapiens
PTKLNAISVLYFDDNSNVILKKYRNMVVR

SEQ ID NO: 64 - protein, 30 aa, Homo sapiens
  VARIANT 7: Xaa = Ser or Thr
PTKLNAXTSVLYFDDNSNVILKKYRNMVVR

SEQ ID NO: 65 - protein, 29 aa, Homo sapiens
  VARIANT 15: Xaa = Ser or Thr
PTKLNAISVLYFNDXSNVILKKYRNMVVR

SEQ ID NO: 66 - protein, 29 aa, Homo sapiens
PTKLNAISVLYFDNNSNVILKKYRNMVVR

SEQ ID NO: 67 - protein, 29 aa, Homo sapiens
  VARIANT 19: Xaa = Ser or Thr
PTKLNAISVLYFDDNSNVXLKKYRNMVVR

SEQ ID NO: 68 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 69 - protein, 20 aa, Homo sapiens
SFRDLGWQDWIIAPEGYAAF

SEQ ID NO: 70 - protein, 20 aa, Homo sapiens
  VARIANT 6: Xaa = Ser or Thr
SFRNLXWQDWIIAPEGYAAF

SEQ ID NO: 71 - protein, 20 aa, Homo sapiens
  VARIANT 11: Xaa = Ser or Thr
SFRDLGWQNWXIAPEGYAAF

SEQ ID NO: 72 - protein, 30 aa, Homo sapiens
YCDGECSFPLNAHMNATNHAIVQTLVHLMF

SEQ ID NO: 73 - protein, 30 aa, Homo sapiens
  VARIANT 13: Xaa = Ser or Thr
YCDGECSFPLNAXMNATNHAIVQTLVHLMF

SEQ ID NO: 74 - protein, 30 aa, Homo sapiens
  VARIANT 20: Xaa = Ser or Thr
YCDGECSFPLNAHMNATNHXIVQTLVHLMF

SEQ ID NO: 75 - protein, 30 aa, Homo sapiens
YCDGECSFPLNAHMNATNHAINQTLVHLMF

SEQ ID NO: 76 - protein, 30 aa, Homo sapiens
PTKLNAISVLYFDDSSNVILKKYRNMVVRS

SEQ ID NO: 77 - protein, 30 aa, Homo sapiens
  VARIANT 7: Xaa = Ser or Thr
PTKLNAXSVLYFDDSSNVILKKYRNMVVRS

SEQ ID NO: 78 - protein, 30 aa, Homo sapiens
PTKLNAISVLYFNDSSNVILKKYRNMVVRS

SEQ ID NO: 79 - protein, 30 aa, Homo sapiens
PTKLNAISVLYFDNSSNVILKKYRNMVVRS

SEQ ID NO: 80 - protein, 30 aa, Homo sapiens
  VARIANT 19: Xaa = Ser or Thr
PTKLNAISVLYFDDSSNVXLKKYRNMVVRS

SEQ ID NO: 81 - protein, 110 aa, Homo sapiens
RNLGLDCDEHSTESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 82 - protein, 110 aa, Homo sapiens
RDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 83 - protein, 110 aa, Homo sapiens
RPSKNKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 84 - protein, 111 aa, Homo sapiens
RSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 85 - protein, 109 aa, Homo sapiens
RNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 86 - protein, 115 aa, Homo sapiens
RAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 87 - protein, 117 aa, Homo sapiens
REKQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 88 - protein, 105 aa, Homo sapiens
KNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 89 - protein, 118 aa, Homo sapiens
RMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 90 - protein, 118 aa, Homo sapiens
RVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 91 - protein, 118 aa, Homo sapiens
RMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 92 - protein, 119 aa, Homo sapiens
RLPGIFDDVHDSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 93 - protein, 120 aa, Homo sapiens
RDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 94 - protein, 112 aa, Homo sapiens
RKQWDEPRVCSRRYLKVDFADIGWNEWIISPKSFDAYYCAGACEFPMPKIVRPSNHATIQSIVRAVGIIPGIPEPCCVPDKMNSLGVLFLDENRNVVLKVYPNMSVDTCACR

SEQ ID NO: 95 - protein, 111 aa, Homo sapiens
KQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 96 - protein, 111 aa, Homo sapiens
RHHLPDRSQLCRKVKFQVDFNLIGWGSWIIYPKQYNAYRCEGECPNPVGEEFHPTNHAYIQSLLKRYQPHRVPSTCCAPVKTKPLSMLYVDNGRVLLDHHKDMIVEECGCL

SEQ ID NO: 97 - protein, 117 aa, Homo sapiens
RGIDCQGGSRMCCRQEFFVDFREIGWHDWIIQPEGYAMNFCIGQCPLHIAGMPGIAASFHTAVLNLLKANTAAGTTGGGSCCVPTARRPLSLLYYDRDSNIVKTDIPDMVVEACGCS

SEQ ID NO: 98 - protein, 115 aa, Homo sapiens
RTPTCEPATPLCCRRDHYVDFQELGWRDWILQPEGYQLNYCSGQCPPHLAGSPGIAASFHSAVFSLLKANNPWPASTSCCVPTARRPLSLLYLDHNGNVVKTDVPDMVVEACGCS

SEQ ID NO: 99 - protein, 116 aa, Homo sapiens
RGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 100 - protein, 127 aa, Homo sapiens
RQADGISAEVTASSSKHSVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 101 - protein, 113 aa, Homo sapiens
RALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 102 - protein, 113 aa, Homo sapiens
RALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 103 - protein, 113 aa, Homo sapiens
RALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 104 - protein, 122 aa, Homo sapiens
GISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 105 - protein, 122 aa, Homo sapiens
GPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 106 - protein, 95 aa, Homo sapiens
GTRCCRQEMYIDLQGMKWAENWVLEPPGFLAYECVGTCRQPPEALAFKWPFLGPRQCIASETDSLPMIVSIKEGGRTRPQVVSLPNMRVQKCSCA

SEQ ID NO: 107 - protein, 102 aa, Homo sapiens
DGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 108 - protein, 115 aa, Homo sapiens
RARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 109 - protein, 101 aa, Homo sapiens
GRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 110 - protein, 93 aa, Homo sapiens
SGPCQLWSLTLSVAELGLGYASEEKVIFRYCAGSCPRGARTQHGLALARLQGQGRAHGGPCCRPTRYTDVAFLDDRHRWQRLPQLSAAACGCG

SEQ ID NO: 111 - protein, 98 aa, Homo sapiens
ARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 112 - protein, 96 aa, Homo sapiens
NRGCVLTAIHLNVTDLGLGYETKEELIFRYCSGSCDAAETTYDKILKNLSRNRRLVSDKVGQACCRPAFDDDLSFLDDNLVYHILRKHSAKRCGCI